SEQUENCE LISTING (63 sequences)

SEQ ID NO: 1 - protein, 510 aa, homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 2 - protein, 472 aa, homo sapiens
MAGKVRSLLPPLLLAAAGLAGLLLLCVPTRDVREPPALKYGIVLDAGSSHTSMFIYKWPADKENDTGIVGQHSSCDVPGGGISSYADNPSGASQSLVGCLEQALQDVPKERHAGTPLYLGATAGMRLLNLTNPEASTSVLMAVTHTLTQYPFDFRGARILSGQEEGVFGWVTANYLLENFIKYGWVGRWFRPRKGTLGAMDLGGASTQITFETTSPAEDRASEVQLHLYGQHYRVYTHSFLCYGRDQVLQRLLASALQTHGFHPCWPRGFSTQVLLGDVYQSPCTMAQRPQNFNSSARVSLSGSSDPHLCRDLVSGLFSFSSCPFSRCSFNGVFQPPVAGNFVAFSAFFYTVDFLRTSMGLPVATLQQLEAAAVNVCNQTWAQQLLSRGYGFDERAFGGVIFQKKAADTAVGWALGYMLNLTNLIPADPPGLRKGTDFSSWVVLLLLFASALLAALVLLLRQVHSAKLPSTI

SEQ ID NO: 3 - protein, 484 aa, homo sapiens
MKKGIRYETSRKTSYIFQQPQHGPWQTRMRKISNHGSLRVAKVAYPLGLCVGVFIYVAYIKWHRATATQAFFSITRAAPGARWGQQAHSPLGTAADGHEVFYGIMFDAGSTGTRVHVFQFTRPPRETPTLTHETFKALKPGLSAYADDVEKSAQGIRELLDVAKQDIPFDFWKATPLVLKATAGLRLLPGEKAQKLLQKVKEVFKASPFLVGDDCVSIMNGTDEGVSAWITINFLTGSLKTPGGSSVGMLDLGGGSTQIAFLPRVEGTLQASPPGYLTALRMFNRTYKLYSYSYLGLGLMSARLAILGGVEGQPAKDGKELVSPCLSPSFKGEWEHAEVTYRVSGQKAAASLHELCAARVSEVLQNRVHRTEEVKHVDFYAFSYYYDLAAGVGLIDAEKGGSLVVGDFEIAAKYVCRTLETQPQSSPFSCMDLTYVSLLLQEFGFPRSKVLKLTRKIDNVETSWALGAIFHYIDSLNRQKSPAS

SEQ ID NO: 4 - protein, 529 aa, homo sapiens
MFTVLTRQPCEQAGLKALYRTPTIIALVVLLVSIVVLVSITVIQIHKQEVLPPGLKYGIVLDAGSSRTTVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEPPVFVGTLAFFTAAALLCLAFLAYLCSATRRKRHSEHAFDHAVDSD

SEQ ID NO: 5 - protein, 428 aa, homo sapiens
MATSWGTVFFMLVVSCVCSAVSHRNQQTWFEGIFLSSMCPINVSASTLYGIMFDAGSTGTRIHVYTFVQKMPGQLPILEGEVFDSVKPGLSAFVDQPKQGAETVQGLLEVAKDSIPRSHWKKTPVVLKATAGLRLLPEHKAKALLFEVKEIFRKSPFLVPKGSVSIMDGSDEGILAWVTVNFLTGQLHGHRQETVGTLDLGGASTQITFLPQFEKTLEQTPRGYLTSFEMFNSTYKLYTHSYLGFGLKAARLATLGALETEGTDGHTFRSACLPRWLEAEWIFGGVKYQYGGNQEGEVGFEPCYAEVLRVVRGKLHQPEEVQRGSFYAFSYYYDRAVDTDMIDYEKGGILKVEDFERKAREVCDNLENFTSGSPFLCMDLSYITALLKDGFGFADSTVLQLTKKVNNIETGWALGATFHLLQSLGISH

SEQ ID NO: 6 - protein, 121 aa, mus musculus
QIQLVQSGPELKKPGETVKISCKASGYTFRNYGMNWVKQAPGKGLKWMGWINTYTGEPTYADDFKGRFAFSLATSASTAYLQISNLKNEDTATYFCARKAYYGSNYYFDYWGQGTTLTVSS

SEQ ID NO: 7 - protein, 108 aa, Mus musculus
DIVMTQSHKFMSTSVGDRVSITCKASQDVSTAVAWYQQKPGQSPKLLIYSASYRYTGVPDRFTGSGSGTDFTFTISTVQAEDLAVYYCQQHYTTPPYTFGGGTKLEIK

SEQ ID NO: 8 - protein, 123 aa, Mus musculus
QIQLVQSGPEVKKPRETVKISCKASGYTFTHYGMNWVKQAPGKGLKWMGWINTYTGEPTYADDFKGRFAFSLEASASTAYLQINNLKNEDTATYFCARRRYEGNYVFYYFDYWGQGTTLTVSS

SEQ ID NO: 9 - protein, 107 aa, Mus musculus
DIQMTQSPASLSASVGETVTITCRASENIYSYFSWYQQKQGKSPQLLVYTAKTLAEGVPSRFSGSGSGTQFSLKINSLQPEDFGSYYCQHHYVTPYTFGGGTKLEIK

SEQ ID NO: 10 - protein, 121 aa, Mus musculus
QVQLVQSGSELKKPGASVKISCKASGYTFTHYGMNWVRQAPGQGLEWMGWINTYTGELTYADDFKGRFVFSLDTSVSTAYLQISSLKAEDTAVYYCARRAYYRYDYVMDYWGQGTLVTVSS

SEQ ID NO: 11 - protein, 107 aa, Mus musculus
DIQMTQSPSSLSASVGDRVTITCKASHNVGTNVAWFQQKPGKAPKSLIYSASYRYSGVPSRFSGSGSGTDFTLTISSLQPEDFATYYCHQYNNYPYTFGQGTKLEIK

SEQ ID NO: 12 - protein, 13 aa, Mus musculus
  misc_feature (2)..(11): Xaa can be any naturally occurring amino acid
  misc_feature (13)..(13): Xaa can be any naturally occurring amino acid
YXXXXXXXXXXSX

SEQ ID NO: 13 - protein, 8 aa, Mus musculus
  misc_feature (2)..(3): Xaa can be any naturally occurring amino acid
  misc_feature (5)..(8): Xaa can be any naturally occurring amino acid
WXXTXXXX

SEQ ID NO: 14 - protein, 23 aa, Mus musculus
  misc_feature (2)..(3): Xaa can be any naturally occurring amino acid
  misc_feature (5)..(10): Xaa can be any naturally occurring amino acid
  misc_feature (12)..(21): Xaa can be any naturally occurring amino acid
  misc_feature (23)..(23): Xaa can be any naturally occurring amino acid
WXXTXXXXXXYXXXXXXXXXXSX

SEQ ID NO: 15 - protein, 5 aa, Mus musculus
  MISC_FEATURE (1)..(5): Two or more of Xaa are aromatic residues
XXXXX

SEQ ID NO: 16 - protein, 14 aa, Mus musculus
  MISC_FEATURE (1)..(1): Xaa is Arg, Lys or Ala
  misc_feature (2)..(4): Xaa can be any naturally occurring amino acid
  MISC_FEATURE (5)..(5): Xaa is Gly, Arg or Asp
  misc_feature (6)..(9): Xaa can be any naturally occurring amino acid
  MISC_FEATURE (10)..(10): Xaa is Tyr, Phe, or Met
  misc_feature (11)..(14): Xaa can be any naturally occurring amino acid
XXXXXXXXXXXXXX

SEQ ID NO: 17 - protein, 11 aa, Mus musculus
  misc_feature (1)..(7): Xaa can be any naturally occurring amino acid
  MISC_FEATURE (8)..(8): Xaa is Ser or Thr
  MISC_FEATURE (9)..(9): Xaa is Tyr, Ala or Asp
  misc_feature (10)..(10): Xaa can be any naturally occurring amino acid
  MISC_FEATURE (11)..(11): Xaa is Ser, His or Ala
XXXXXXXXXXX

SEQ ID NO: 18 - protein, 7 aa, Mus musculus
  misc_feature (1)..(7): Xaa can be any naturally occurring amino acid
XXXXXXX

SEQ ID NO: 19 - protein, 7 aa, Mus musculus
  MISC_FEATURE (7)..(7): Xaa is Thr or Ser
SASYRYX

SEQ ID NO: 20 - protein, 10 aa, Mus musculus
  MISC_FEATURE (1)..(1): Xaa is Glu or His
  MISC_FEATURE (2)..(2): Xaa is Glu or His
  MISC_FEATURE (3)..(3): Xaa is Tyr, His or Thr
  misc_feature (4)..(10): Xaa can be any naturally occurring amino acid
XXXXXXXXXX

SEQ ID NO: 21 - protein, 330 aa, artificial
  Modified homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, artificial
  Modified homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 330 aa, artificial
  Modified homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 330 aa, artificial
  Modified homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 5 aa, Mus musculus
NYGMN

SEQ ID NO: 26 - protein, 17 aa, Mus musculus
WINTYTGEPTYADDFKG

SEQ ID NO: 27 - protein, 6 aa, mus musculus
RFAFSL

SEQ ID NO: 28 - protein, 6 aa, homo sapiens
RFVFSL

SEQ ID NO: 29 - protein, 12 aa, Mus musculus
KAYYGSNYYFDY

SEQ ID NO: 30 - protein, 11 aa, Mus musculus
KASQDVSTAVA

SEQ ID NO: 31 - protein, 7 aa, Mus musculus
SASYRYT

SEQ ID NO: 32 - protein, 10 aa, Mus musculus
QQHYTTPPYT

SEQ ID NO: 33 - protein, 5 aa, Mus musculus
HYGMN

SEQ ID NO: 34 - protein, 14 aa, Mus musculus
RRYEGNYVFYYFDY

SEQ ID NO: 35 - protein, 11 aa, Mus musculus
RASENIYSYFS

SEQ ID NO: 36 - protein, 7 aa, Mus musculus
TAKTLAE

SEQ ID NO: 37 - protein, 9 aa, Mus musculus
QHHYVTPYT

SEQ ID NO: 38 - protein, 17 aa, Mus musculus
WINTYTGELTYADDFKG

SEQ ID NO: 39 - protein, 12 aa, Mus musculus
RAYYRYDYVMDY

SEQ ID NO: 40 - protein, 11 aa, Mus musculus
KASHNVGTNVA

SEQ ID NO: 41 - protein, 7 aa, Mus musculus
SASYRYS

SEQ ID NO: 42 - protein, 9 aa, Mus musculus
HQYNNYPYT

SEQ ID NO: 43 - protein, 5 aa, Mus musculus
  MISC_FEATURE (1)..(1): Xaa is His or Asp
  misc_feature (2)..(2): Xaa can be any naturally occurring amino acid
XXGMN

SEQ ID NO: 44 - protein, 8 aa, mus musculus
WINTYTGE

SEQ ID NO: 45 - protein, 13 aa, mus musculus
YADDFKGRFAFSL

SEQ ID NO: 46 - protein, 13 aa, artificial
  Mus muculus-homo sapiens chimeric
YADDFKGRFVFSL

SEQ ID NO: 47 - protein, 6 aa, Mus musculus
  misc_feature (1)..(1): Xaa can be any naturally occurring amino acid
  misc_feature (3)..(4): Xaa can be any naturally occurring amino acid
  misc_feature (6)..(6): Xaa can be any naturally occurring amino acid
XFXXSX

SEQ ID NO: 48 - protein, 12 aa, Mus musculus
RRYEGNYVFYYF

SEQ ID NO: 49 - protein, 10 aa, Mus musculus
KAYYGSNYYF

SEQ ID NO: 50 - protein, 10 aa, Mus musculus
RAYYRYDYVM

SEQ ID NO: 51 - protein, 7 aa, Mus musculus
RASENIY

SEQ ID NO: 52 - protein, 7 aa, Mus musculus
KASQDVS

SEQ ID NO: 53 - protein, 7 aa, mus musculus
KASHNVG

SEQ ID NO: 54 - protein, 8 aa, Mus musculus
  MISC_FEATURE (1)..(1): Xaa is any aromatic residue
  MISC_FEATURE (2)..(2): Xaa is Thr or Ser
  misc_feature (3)..(8): Xaa can be any naturally occurring amino acid
XXXXXXXX

SEQ ID NO: 55 - protein, 8 aa, mus musculus
YTAKTLAE

SEQ ID NO: 56 - protein, 8 aa, mus musculus
YSASYRYT

SEQ ID NO: 57 - protein, 8 aa, mus musculus
YSASYRYS

SEQ ID NO: 58 - protein, 9 aa, mus musculus
  misc_feature (2)..(2): Xaa can be any naturally occurring amino acid
  misc_feature (4)..(5): Xaa can be any naturally occurring amino acid
QXHXXTPYT

SEQ ID NO: 59 - protein, 5 aa, homo sapiens
FVFSL

SEQ ID NO: 60 - DNA, 45 bp, artificial
  Primer
tacgactcacaagcttgccgccaccatggaagatacaaaggagtc

SEQ ID NO: 61 - DNA, 64 bp, artificial
  Primer
ccgccccgactctagatcacttgtcatcgtcatctttgtaatcgacataggtggagtgggagag

SEQ ID NO: 62 - protein, 451 aa, artificial
  Chimeric
QIQLVQSGPELKKPGETVKISCKASGYTFRNYGMNWVKQAPGKGLKWMGWINTYTGEPTYADDFKGRFAFSLATSASTAYLQISNLKNEDTATYFCARKAYYGSNYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 215 aa, artificial
  Chimeric
DIVMTQSHKFMSTSVGDRVSITCKASQDVSTAVAWYQQKPGQSPKLLIYSASYRYTGVPDRFTGSGSGTDFTFTISTVQAEDLAVYYCQQHYTTPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0153113 A1
(43) Pub. Date: May 23, 2019

(54) CD39 VASCULAR ISOFORM TARGETING AGENTS

(71) Applicants: INNATE PHARMA, Marseille (FR); OREGA BIOTECH, Ecully (FR)

(72) Inventors: Jérémy BASTID, Francheville (FR); Laurent GAUTHIER, Marseille (FR); Carine PATUREL, Marcy l'Etoile (FR); Ivan PERROT, Roquefort La Bédoule (FR); Alain ROUSSEL, Marseille (FR); Béatrice AMIGUES, Saint Maximin la Sainte Baume (FR)

(21) Appl. No.: 15/778,202

(22) PCT Filed: Nov. 22, 2016
(86) PCT No.: PCT/EP2016/078395

§ 371 Date: May 22, 2018

Related U.S. Application Data
(60) Provisional application No. 62/258,701, filed on Nov. 23, 2015.
(60) Provisional application No. 62/263,760, filed on Dec. 7, 2015.
(60) Provisional application No. 62/267,343, filed on Dec. 15, 2015.
(60) Provisional application No. 62/320,738, filed on Apr. 11, 2016.
(60) Provisional application No. 62/404,779, filed on Oct. 6, 2016.

(51) Int. Cl. C07K 16/28; A61P 35/00

(52) U.S. Cl. CPC C07K 16/2896; A61P 35/00; C07K 16/2818; C07K 2317/76; C07K 2317/565; C07K 2317/567; C07K 2317/33; C07K 2317/92; C07K 2317/34; C07K 2317/71; C07K 2317/94; C07K 2317/24; C07K 2317/52

(57) ABSTRACT

The present invention relates to antigen-binding compounds that inhibit CD39. The invention also relates to cells producing such compounds; methods of making such compounds, and antibodies, fragments, variants, and derivatives thereof; pharmaceutical compositions comprising the same; methods of using the compounds to diagnose, treat or prevent diseases, e.g. cancer.

### CROSS-REFERENCE TO RELATED APPLICATIONS

This application is a U.S. National Stage Application of International Application NO. PCTEP2016/078395, filed on Nov. 22, 2016, designating the U.S. and published in English as WO 2017/089334 A1 on Jun. 1, 2017, which claims the benefit of U.S. Provisional Application Nos. U.S. 62/258,701 filed 23 Nov. 2015, U.S. 62/263,760 filed 7 Dec. 2015, U.S. 62/267,343 filed 15 Dec. 2015, U.S. 62/320,738 filed 11 Apr. 2016, and U.S. 62/404,779 filed 6 Oct. 2016, the disclosures of which are incorporated herein by reference in their entireties, including any drawings. Any and all applications for which a foreign and/or a domestic priority is claimed is/are identified in the Application Data Sheet filed herewith and is/are hereby incorporated by reference in their entireties under 37 C.F.R. § 1.57.

### REFERENCE TO SEQUENCE LISTING

The present application is being filed along with a Sequence Listing in electronic format. The Sequence Listing is provided as a file entitled “CD39-1_ST25”, created 21 Nov. 2016, which is 55 KB in size. The information in the electronic format of the Sequence Listing is incorporated herein by reference in its entirety.

### FIELD OF THE INVENTION

The present invention relates to antigen-binding compounds (e.g. antibodies) that inhibit CD39. The invention also relates to cells producing such compounds; methods of making such compounds, and antibodies, fragments, variants, and derivatives thereof; pharmaceutical compositions comprising the same; methods of using the compounds to diagnose, treat or prevent diseases, e.g. cancer.

### BACKGROUND

Eight different ENTPD genes encode members of the NTPDase protein family. The individual NTPDase subtypes differ in cellular location and functional properties. Plasma membrane-bound nucleoside triphosphate diphosphohydrolases control nucleotide levels at the cell surface by hydrolyzing the c and b phosphates of nucleotides.

NTPDase 1 (ectonucleoside triphosphate diphosphohydrolase1), also known as CD39/ENTPD1 or vascular CD39, functions together with another enzyme, CD73 (ecto-5′-nucleotidase), to hydrolyze extracellular adenosine triphosphate (ATP) and adenosine diphosphate (ADP) to generate adenosine, which binds to adenosine receptors and inhibits T-cell and natural killer (NK)-cell responses, thereby suppressing the immune system. The generation of adenosine via the CD73/CD39 pathway is recognized as a major mechanism of regulatory T cell (Treg) immunosuppressive function. The number of CD39⁺ Tregs is increased in some human cancers, and the importance of CD39⁺ Tregs in promoting tumor growth and metastasis has been demonstrated using several in vivo models. However, CD39 is also expressed by tumor cells and CD39⁺ tumor cells can mediate immunosuppression via the adenosine pathway. CD39 in cancer cells displays ATPase activity and, together with CD73, generates adenosine. CD73⁺CD39⁺ cancer cells inhibited the proliferation of CD4 and CD8 T cells and the generation of cytotoxic effector CD8 T cells (CTL) in a CD39− and adenosine-dependent manner. Antibodies that bind and inhibit CD39 antibodies are disclosed in WO2009/095478. Hayes et al. (2015) Am. J. Transl. Res. 7(6):1181-1188 makes use of an anti-CD39 that binds FcγR and has effector function but it stated to also be blocking.

Despite the long-standing interest in CD39 as a therapeutic target, the characteristics of the most effective anti-CD39 antibodies remains to be determined. The NTPDase family includes at least 8 members which differ in their expression and substrate preference. There exist, notably, within the NTPDase family, several CD39 isoforms, including vascular CD39, CD39L1 (NTPDase2), CD39L2 (NTPDase6), CD39L3 (NTPDase3) and CD39L4 (NTPDase5). The CD39, CD39-L1, and CD39-L3 genes encode hydrophobic portions in their carboxy and amino termini, serving as transmembrane domains that anchor the CD39 enzyme to the surface of cells and position the enzymatic activity outside the cell. The CD39-L2 and CD39-L4 genes encode hydrophobic portions in their amino termini, consistent with presence in secreted, soluble form. See, e.g., Yeung et al., (2000) Biochem. 39:12916-12923. CD39 is generally referred to as “vascular” CD39, a membrane bound protein expressed, inter alia, on endothelial cells and was initially described as having a role in modulating circulating levels of nucleotides in the blood. CD39L2 and CD39L4 represent a type of CD39 which can be present as both membrane-bound and soluble form. Specificity for hydrolysis of ADP over ATP has been reported to implicate these L2 and L4 forms as possible regulators that have a role in preventing excessive platelet aggregation that could lead to thrombosis. The membrane bound isoform CD39-L3 has been reported to be the major ectonucleotidase in pancreatic β-cells that can regulate insulin secretion (Syed et al. (2013) Endocrin. Metabol. 305(10):E1319-1326).

Consequently, CD39 expression on different cell types, including leukocytes and tumor cells, combined with use of antibodies that either do not actually block CD39 or are not pure blockers, create a complex setting for evaluation of the underlying activity of antibodies.

### SUMMARY OF THE INVENTION

The inventors have discovered antibodies that bind an epitope present on human CD39 expressed at the surface of cells, including tumor cells, and that potently inhibit the enzymatic (ATPase activity) activity of the CD39 enzyme, including cell surface (membrane bound) enzyme, moreover without dependence on ability to substantially induce or increase CD39 internalization.

CD39 is widely expressed within human tissues, implying that maintaining continuous antibody-mediated receptor saturation may be challenging. By avoiding induction of receptor internalization, the antibodies reduce the re-cycling of free CD39 to the cell surface, in turn reducing the concentration of antibody required to maintain saturation of cell surface CD39. The antibodies of the disclosure thereby enable methods of treatment (e.g. of individuals having cancer, infectious disease), wherein an anti-CD39 antibody is administered (e.g. by intravenous administration) at lower frequencies, e.g. less than daily. For example the antibody can be administered (e.g. by intravenous administration) about once every week, once every two weeks, 1-4 times per month, 1-2 times per month, 1-2 times every two months, or less frequently.

Provided in another aspect are assay methods for identifying antibodies that potently inhibit the enzymatic (ATPase activity) activity of the CD39 enzyme, including cell surface (membrane bound) enzyme, moreover without dependence on ability to inducing or increasing receptor internalization.

In another embodiment, the inventors have determined the co-crystal structure of the antibodies (as Fab fragment) with CD39, thereby identifying key structural features underlying the mechanism of action of the antibodies by which the antibody is capable of binding to the N-terminal domain of CD39 and the C-terminal domain of CD39, e.g., to inhibit the domain movement of the cell surface CD39 polypeptide. The disclosure of such structural features enables the modification of antibodies while maintaining functionality in CD39 inhibition.

In yet a further embodiment, provided are anti-CD39 antibodies whose key structural features include a V_{H} CDR3 comprising a plurality of aromatic amino acid residues.

In yet a further embodiment the antibody comprises a V_{H} and V_{L}, wherein the V_{H} comprises a Kabat CDR3 comprising at least one first aromatic amino acid residue capable of interacting with a residue of the V_{L} and at least one second aromatic amino acid residue capable of interacting with a residue of CD39. Optionally, the first and second aromatic residues are each independently a tyrosine or a phenylalanine. Advantageously, the antibodies can comprise an Fc domain comprising one of more amino acid modifications (e.g. substitutions) that enhance the in vitro and/or in vivo stability of the antibody.

In yet a further embodiment, the disclosure provides modified human IgG1 Fc domains that confer increased physical stability (e.g. in a pharmaceutical formulation) to an antibody characterized by high hydrophobicity (e.g. predicted hydrophobicity) and/or by the presence of a plurality of surface exposed aromatic amino acid residues in their CDRs. While these modified Fc domain can be used to improve the stability the anti-CD39 antibodies described herein that comprise aromatic amino acid residue in their Kabat CDR3, it will be appreciated that the modified Fc domains can also be used to increase the stability of antibodies that bind antigens other than CD39. For example, the modified Fc domains can be used to increase the stability of non-depleting antibodies, for example antibodies that bind a soluble or cell-expressed protein without a need to mediate effector function (e.g. ADCC), for example function-blocking antibodies or antibody-drug conjugates. In one embodiment, provided is a monoclonal antibody comprising a plurality of aromatic amino acid residues in one or more CDRs (e.g. V_{H} CDR2 and/or V_{H} CDR3), wherein the antibody comprises a modified human IgG1 Fc domain that confers increased physical stability and/or solubility (e.g. in a pharmaceutical formulation) to the antibody. Such modified Fc domain can be particularly useful in antibodies having extended V_{H} CDR3s, e.g. comprising a Kabat CDR3 of 9, 10, 11, 12, 13 or 14 amino acids in length, or comprising an amino acid residue present at one or more (e.g. 2, 3, 4, 5 or 6 or 7) of Kabat positions 100a to 100f. In one embodiment, a CDR (e.g. the V_{H} CDR3) of the antibody comprises a sequence of amino residues having the formula X₁ X₂ X₃ X₄ X₅ (SEQ ID NO: 5), wherein any two, three or more of X₁, X₂, X₃, X₄ and X₅ represent an aromatic amino acid, optionally a tyrosine or a phenylalanine. In one embodiment, provided is a monoclonal antibody comprising a heavy chain comprising a Kabat V_{H} CDR2 comprising three, four or more aromatic acid residues. In one embodiment, provided is a monoclonal antibody comprising a heavy chain comprising a Kabat V_{H} CDR3 comprising three, four, five, six or seven (or more) aromatic acid residues. In one embodiment, provided is a monoclonal antibody comprising a heavy chain comprising a Kabat V_{H} CDR3 comprising three, four, five, six or seven (or more) aromatic acid residues, and an Fc domain of human IgG1 isotype comprising an amino acid substitution in a heavy chain constant region (e.g. compared to a reference Fc domain, e.g. a wild type human IgG1 Fc domain) at Kabat positions 234, 235 and 331, optionally at Kabat positions 234, 235, 237 and 331, or optionally at Kabat positions 234, 235, 237, 330 and 331. In one embodiment, the modified Fc domain comprises an amino acid sequence of any one of SEQ ID NOS: 21, 22, 23 or 24. In one embodiment, the aromatic acid residues are selected from tyrosine and phenylalanine. In one embodiment, the VH comprises human framework amino acid sequences. In one embodiment, the antibody comprises a light chain comprising a VL, optionally a VL comprising human framework amino acid sequences. In one embodiment, the antibody is a full-length IgG antibody comprising two light chain and two heavy chains. In one embodiment, provided is a pharmaceutical formulation comprising such antibody.

Provided in one aspect are anti-CD39 antibodies capable of binding to and inhibiting the activity of a human CD39 polypeptide, the antigen-binding protein comprising a V_{H} and a V_{L} that each comprise a framework (e.g. a framework having an amino acid sequence of human origin) and a CDR1, CDR2 and CDR3, wherein the antigen-binding protein is capable of binding to the N-terminal domain of CD39 and the C-terminal domain of CD39. In one embodiment, the antigen-binding protein restricts the domain movement of CD39 when bound to CD39. Optionally, the V_{H} and/or V_{L} framework (e.g. FR1, FR2, FR3 and/or FR4) is of human origin. In one embodiment, the V_{H} comprises a first CDR (or antigen binding domain) that is capable of binding to the N-terminal domain of CD39 and a second CDR (or antigen binding domain) that is capable of binding to amino acid residues of the C-terminal domain of CD39.

In one aspect of the invention (e.g. in any aspect herein), an anti-CD39 antibody comprises a V_{H} and a V_{L} domain each comprising a CDR1, CDR2 and CDR3, wherein the Kabat CDR2 (optionally together with the FR3) of the V_{H} binds to amino acid residues and/or to the N292-linked glycan in the C-terminal domain of CD39. Optionally, the Kabat CDR1 of the V_{H} binds to amino acid residues in the N-terminal domain of CD39. Optionally, the Kabat CDR3 of the V_{H} binds to amino acid residues the N-terminal domain of CD39. Optionally the CDR2 of the V_{H} comprises a first amino acid segment that binds to the N-terminal domain of CD39 together and an amino acid segment that binds, together with FR3 residues, to the C-terminal domain of CD39. Optionally, the CDR3 comprises an aromatic residue (e.g. a tyrosine) that is capable of binding an amino acid residue in the N-terminal domain of CD39 and a second aromatic amino acid residue (e.g., a tyrosine, a phenylalanine) that is capable of contacting an amino acid residue in the V_{L}. Optionally, the binding molecule or antigen-binding fragment comprises a V_{L} that binds, via a residue in a Kabat CDR, to the Kabat CDR3 of the V_{H}.

Provided in one aspect are compositions (e.g., binding molecules) and methods for substantially completely inhibiting, in vivo or in vitro, the ATPase activity of cellular CD39 with a pure antagonist, or e.g., an agent that lacks effector function, pro-apoptotic activity, or toxin linkage. In one embodiment, the antagonist (e.g. anti-CD39 antibody) is administered at less than daily frequencies, optionally less than weekly frequency, e.g. less than daily, once about every week, once every two weeks, 1-4 times per month, 2-4 times per month, 1-2 times per month, 1-2 times every two months, or less frequently.

Provided in one aspect are methods for modulating the ability of anti-CD39 antibodies to undergo intracellular internalization and/or induce or increase receptor internalization in CD39-expressing cells. Also provided are antigen binding molecules (including antigen-binding fragments thereof) having modified ability to cause CD39 internalization on cells, notably in immune cells (e.g. B cells, T cells) and tumor cells.

While CD39 is expressed on tumor cells (in addition to immune cells), CD39 can also be advantageously targeted for immunomodulation (on tumor cell and immune cells). The CD39-binding molecules provided are particularly advantageous as a medicament destined to act as a pure inhibitor of CD39, e.g., by decrease CD39 ATPase activity in a cell without conjugation to a cytotoxic agent, inducing apoptosis or induction of ADCC toward a CD39-expressing cell.

The antibody does not induce or increase CD39 down-modulation on cells, despite retaining the ability to bind CD39 polypeptides in bivalent manner (the antibody employed in the Examples has two antigen binding domains that are each capable of binding a CD39 polypeptide). Advantageously, in one embodiment the antibody comprises a human Fc domain that is modified to have decreased or substantially lack binding to a human Fcγ receptor, e.g. one or more (or all of) human CD16, CD32a, CD32b and CD64, thereby eliminating potential induction of CD39 down-modulation (e.g., in vivo; in the presence of Fcγ receptor-expressing cells). The property of non-internalization and non-down-modulation can confer an improved pharmacology in vivo, in turn leading to a more complete neutralization of CD39 activity in vivo. In one embodiment, the binding molecule (e.g. antibody) comprises the variable heavy chain domain (V_{H}) comprising a CDR1, 2 and 3 as described herein, and a variable light chain domain (V_{L}) comprising a CDR1, 2 and 3 as described herein. In one embodiment, the binding molecule (e.g. antibody) comprises the variable heavy chain domain (V_{H}) of formula I and a variable light chain domain (V_{L}) of formula II.

In alternative embodiment, the binding molecule can be produced such that it retains and/or mediates effector function via its Fc domain. In one embodiment the antibody comprises a human Fc domain that binds to a human Fcγ receptor, e.g. one or more (or all of) human CD16, CD32a, CD32b and CD64.

In another embodiment, the Fc domain can be modified to reduce Fcγ receptor binding, optionally by retaining binding to one or more human Fcγ receptor(s) but having decreased binding to one or more other human Fcγ receptor(s).

In one aspect of any embodiment herein, the binding molecule (e.g., antibody) comprises a variable light chain domain (V_{H}) CDR1, CDR2 and/or CDR3 described herein. In one aspect of any embodiment herein, the binding molecule (e.g., antibody) comprises a variable light chain domain (V_{L}) CDR1, CDR2 and/or CDR3 described herein.

In one aspect of any embodiment herein, the binding molecule (e.g., antibody) comprises the variable light chain domain (V_{H}) described herein a variable heavy chain domain (V_{L}) described herein. In one aspect of any embodiment herein, the binding molecule (e.g., antibody) comprises the variable light chain domain (V_{H}) of formula I and a variable heavy chain domain (V_{L}) of formula II as described herein.

In one aspect, provided is an antibody or antibody fragment comprising a V_{H} that binds CD39 and a V_{L} that binds to the Kabat CDR3 of the V_{H}, optionally wherein the Kabat CDR3 of the V_{H} is an extended V_{H} CDR3, e.g. comprising a Kabat CDR3 of 9, 10, 11, 12, 13 or 14 amino acids in length, or comprising an amino acid residue present at one or more (e.g. 2, 3, 4, 5 or 6 or 7) of Kabat positions 100a to 100f, wherein the Kabat CDR3 of the V_{H} comprises at least 2, 3, 4, 5, 6 or more aromatic amino acid residues. In one embodiment, the aromatic amino acid residues are independently selected from tyrosine and phenylalanine. In one embodiment, the Kabat CDR3 of the V_{H} comprises at least 2, 3, 4, 5, 6 or more tyrosine residues. In one embodiment, the Kabat CDR3 of the V_{H} comprises a first aromatic amino acid residue that is capable of contacting an amino acid residue in CD39 and a second aromatic amino acid residue that is capable of contacting an amino acid residue in the V_{L}.

In one aspect, an antibody or antibody fragment comprises a V_{H} that binds CD39 and a V_{L} that binds the CDR3 of the V_{H}, wherein the V_{H} comprises:
(a) a CDR1 capable of contacting the N-terminal domain of CD39, optionally comprising a residue at Kabat position 33, optionally at both positions 31 and 33, that is capable of contacting amino acid residues in CD39;
(b) a CDR2-FR3 domain comprising:
   a. optionally, a segment comprising amino acid residues capable of contacting the N-terminal domain of CD39, optionally wherein the segment comprises residues within Kabat positions 50-56, optionally wherein the segment comprises one, two, three, four or more of (or all of) the residues at Kabat positions 50, 52, 52a, 53 and/or 56, optionally wherein the residue at position 53 aromatic is an amino acid residue;
   b. a segment comprising amino acid residues capable of contacting the C-terminal domain of CD39, optionally wherein the segment comprises residues within Kabat positions 59-71, optionally wherein the segment comprises one, two, three, four or more of (or all of) the residues at Kabat positions 59, 65, 67, 68, 69, 70 and/or 71, optionally wherein the segment further the residue at Kabat position 54, optionally further the residues at Kabat positions 72, 72a and/or 72b; and
(c) a CDR3 (e.g. according to Kabat) capable of contacting the N-terminal of CD39, optionally capable of contacting the N-terminal domain of CD39 and the V_{L}, optionally comprising a first aromatic amino acid residue that is capable of contacting an amino acid residue in CD39 and a second aromatic amino acid residue that is capable of contacting an amino acid residue in the V_{L}, optionally further wherein the first and second aromatic residues are at any of Kabat positions 100, 100b, 100c, 100d, 100e and/or 100f (to the extent a residue is present at the particular Kabat position). Optionally, the antibody or antibody fragment comprises an Fc domain as disclosed herein, e.g., a modified Fc domain that improves antibody stability such as an Fc domain of human IgG1 isotype comprising an amino acid substitution in a heavy chain constant region (e.g. compared to a reference Fc domain, e.g. a wild type human IgG1 Fc domain) at Kabat positions 234, 235 and 331, optionally at Kabat positions 234, 235, 237 and 331, or optionally at Kabat positions 234, 235, 237, 330 and 331.

In one aspect of any embodiment herein, an antibody or antibody fragment comprises a V_{H} comprising:

(a) a CDR1 capable of contacting the N-terminal domain of CD39, optionally wherein the residues at Kabat position 31, 32 and 33 have the formula X₁ X₂ X₃, wherein X₁ represents any amino acid, optionally a histidine or asparagine, X₂ represents any amino acid, optionally an aromatic residue, optionally a tyrosine, or optionally an amino acid residue other than a proline or glycine, and X₃ represents glycine, or another amino acid that avoids steric hindrance;

(b) a CDR2-FR3 segment capable of contacting the C-terminal domain of CD39, optionally wherein the residues at Kabat position 59-71 have the formula X₁ X₂ X₃ X₄ X₅ X₆ X₇ X₈ X₉ X₁₀ X₁₁ X₁₂ X₁₃ (SEQ ID NO: 12), wherein X₁ represents a tyrosine, each of X₂, X₃, X₄, X₅ and X₆ each represent any amino acid, X₇ represents glycine or another residue which does not introduce steric hindrance that reduces antigen binding, X₅ represents any amino acid, X₉ represents phenylalanine or another hydrophobic residue capable of maintaining the beta-strand position and V_{H} domain structure integrity, X₁₀ represents alanine or valine, or optionally leucine, optionally threonine, optionally a hydrophobic residue, X₁₁ represents phenylalanine or another hydrophobic residue (e.g. isoleucine) capable of maintaining the beta-strand position and V_{H} domain structure integrity and X₁₂ represents serine, optionally further wherein and X₁₃ represents any amino acid, optionally leucine, optionally alanine, valine, threonine or arginine, optionally wherein the CDR2-FR3 segment further comprises residues at Kabat positions 72, 72a and 72b having the formula X₂₄ X₂₅ X₂₆, wherein X₂₄ represents aspartic acid, glutamic acid or alanine, X₂₅ represents any amino acid, optionally alanine or threonine, and X₂₆ represents serine, optionally alanine; and

(c) a CDR3 capable of contacting the N-terminal of CD39, optionally wherein the residues at Kabat position 100 to 100f, to the extent residues are present at these positions, comprise a sequence of amino residues having the formula X₁ X₂ X₃ X₄ X₅ (SEQ ID NO: 15), wherein any two, three or more of X₁, X₂, X₃, X₄ and X₅ represent an aromatic amino acid.

In one aspect of any embodiment herein, an antibody or antibody fragment comprises a V_{L} comprising:

a CDR1 wherein the residues at Kabat position 31, 32, 33 and 34 have the formula X₁ X₂ X₃ X₄, wherein X₁ represents a threonine, serine or a conservative substitution thereof, X₂ represents alanine or asparagine, or a conservative substitution thereof, X₃ represents valine or a conservative substitution thereof, and X₄ represents alanine or a conservative substitution thereof;

a FR2 comprising an aromatic residue, optionally a tyrosine, at Kabat position 49;

a CDR2 wherein the residue at Kabat position 50 is a serine, lysine or threonine or a conservative substitution thereof; and

a CDR3 wherein the residues at Kabat position 89 is a glutamine or histidine, or a conservative substitution thereof, the residue at position 91 is a tyrosine, threonine or histidine, or a conservative substitution thereof, optionally wherein the residue at position 95 is a proline, or a conservative substitution thereof, optionally wherein the residue at position 96 is an aromatic residue, optionally a tyrosine or phenylalanine, or a conservative substitution thereof.

The exemplary antibodies can advantageously bind specifically to the cell membrane-bound isoform of CD39 known as “vascular” CD39, but not substantially to other NTPDases, notably the CD39 forms known as CD39-L1, -L2, L3 and/or -L4. The lack of binding to secreted, soluble L2 and L4 isoforms may provide, inter alia, advantageous pharmacological profiles. Avoiding binding to the secreted isoforms as well as to membrane bound L1 and L3 may furthermore help in avoiding undesired side effects of CD39 blockade.

The antibodies of the disclosure can inhibit the enzymatic activity of membrane-bound CD39 protein expressed at the surface of cells, and, in certain embodiments, without substantially inducing or increasing intracellular internalization of, or more generally down-modulation of, cell surface-expressed CD39.

In one aspect, the antibodies do not substantially induce or increase intracellular internalization and therefore do not depend on CD39 down-modulation or ADCC-, CDC- or toxin-mediated depletion of CD39-expressing cells for their CD39 inhibitory activity. These antibodies can be used as “pure” CD39 blockers, targeted to vascular CD39, permitting immunomodulatory activity.

The antibodies of the disclosure can be capable of inhibiting the enzymatic activity of membrane-bound CD39 protein expressed at the surface of cells, with or without induction of CD39 internalization, and with or without binding of CD16 (FcγIII receptor) and/or with or without substantially directing ADCC and/or CDC toward a CD39-expressing cell. Optionally, the antibodies retain an Fc domain and retain binding to human FcRn.

Also provided are methods and assays that have low sensitivity to down-modulation of CD39 expression on cells. Such assays can be used advantageously to screen or test antibodies or other antigen binding agents for their ability to neutralize CD39, and can optionally be useful to separate antibodies that either have or lack the ability undergo internalization, or to increase or induce intracellular internalization of CD39. In one embodiment of such an assay, the method comprises: (i) bringing CD39-expressing cells, optionally Ramos lymphoma cells (e.g. as used in the Examples herein, available for example from the ATCC, reference CRL-1596) into contact with a test antibody (e.g. a plurality of test antibodies), and (ii) assessing production of AMP by mass spectrometry, wherein a decrease in AMP generated (e.g. compared to a negative control, for example an isotype control antibody) indicates neutralization of ATPase activity. Optionally an antibody causes a decrease of AMP generated by at least 70%, 80% or 90% in this assay. Optionally the method further comprises selecting an antibody (e.g. for use in therapy, for production of a batch of antibody, for further processing or evaluation) that results in a decrease of AMP generated by at least 70%, 80% or 90%.

Advantageously, the antibodies exemplified herein target the membrane-bound vascular isoform of CD39 (the polypeptide shown in SEQ ID NO: 1) without binding to a soluble CD39 isoform, e.g. isoforms L2 and/or L4. Additionally, the antibodies exemplified herein furthermore do not bind the L1 and/or L3 isoforms of CD39.

While antibodies that function by inducing ADCC and/or CDC may be efficient even without complete neutralization/inhibition of the ATPase activity of CD39, as long as enough antibody is bound to a CD39-expressing cell to induce ADCC, neutralizing non-depleting antibodies are believed to require strong inhibition of the enzymatic activity of ATPase. In one embodiment, a non-depleting antibody will provide an at least 70%, 80%, 90% reduction in the ATPase activity of a CD39-expressing cell (e.g. as assessed by decrease in AMP generation by a CD39+ cell such as a B cell, a Ramos cell, as measured by mass spectrometry), at a concentration compatible with administration of an antibody to a human. The antibodies identified by these methods were then tested in cellular enzymatic activity assays using purified antibody, and found to strongly neutralize the enzymatic activity of vascular human CD39 (>90% inhibition of AMP generation by B cells (Ramos)). The epitope on CD39 bound by these antibodies is present on CD39 polypeptides as expressed by a range of cells, e.g. cancer cells, CD4 T cells, CD8 T cells, B cells, transfected cells, and binds with high affinity as determined by flow cytometry. For example, an antibody can be characterized by an EC₅₀, as determined by flow cytometry, of no more than 2 μg/ml, no more than 1 μg/ml, no more than 0.5 μg/ml, no more than 0.1 μg/ml or no more than 0.05 μg/ml, for binding to cells that express at their surface a CD39 polypeptide. In one embodiment the cells are cells that are made to express CD39 at their surface. In one embodiment the cells are cells that endogenously express CD39 at their surface, e.g. regulatory T (TReg) cells, B cells, cancer cells, lymphoma cells (e.g. Ramos cells), leukemia cells, bladder cancer cells, glioma cells, glioblastoma cells, ovarian cancer cells, melanoma cells, prostate cancer cells, thyroid cancer cells, esophageal cancer cells or breast cancer cells.

In one aspect, provided is a CD39-binding agent that binds an antigenic determinant present on human “vascular” CD39 (e.g. a polypeptide of SEQ ID NO: 1) but not present on a soluble CD39 isoform, e.g., CD39-L2 and/or -L4. In one aspect, provided is a CD39-binding agent that binds an antigenic determinant present on the “vascular” CD39 (e.g. a polypeptide of SEQ ID NO: 1) but lacking on any one or more (or all of) the L2, L3 and/or L4 isoforms of CD39. Optionally, the CD39-binding agent further binds to cells expressing at their surface human non-human primate CD39 polypeptide (e.g. a cynomolgus monkey CD39 polypeptide).

In one aspect of any embodiment herein, an antibody that binds human CD39 comprises an Fc domain that is modified (compared to a wild-type Fc domain of the same isotype) to reduce binding between the Fc domain and human CD16A, CD16B, CD32A, CD32B and/or CD64 polypeptides, wherein the Fc domain comprises an amino acid substitution (e.g. compared to a reference Fc domain, e.g. a human IgG1 Fc domain) in a heavy chain constant region at Kabat positions 234, 235 and 331, optionally at Kabat positions 234, 235, 237 and 331, or optionally at Kabat positions 234, 235, 237, 330 and 331. In one embodiment, the antibody has an amino acid substitution in a heavy chain constant region at any three, four, five or more of residues selected from the group consisting of: 234, 235, 237, 322, 330 and 331 (Kabat numbering). Optionally, a phenylalanine or an alanine is present at Kabat position 234. Optionally, a glutamic acid is present at position 235. Optionally, an alanine is present at position 237. Optionally, a serine is present at position 330. Optionally, a serine is present at position 331. In one embodiment, the V_{H} CDR3 of the antibody comprises a plurality of surface-exposed aromatic residues, optionally, a Kabat V_{H} CDR3 may comprise a sequence of amino residues having the formula X₁ X₂ X₃ X₄ X₅ (SEQ ID NO: 5), wherein any three or more of X₁, X₂, X₃, X₄ and X₅ represent an aromatic amino acid. Optionally, at least three of the aromatic residues are tyrosines. Optionally at least two aromatic residues are tyrosines and at least one aromatic residue is a phenylalanine. Optionally, at least one of the aromatic residues in V_{H} CDR3 is capable of interacting with CD39, optionally further wherein at least one of the aromatic amino acids within V_{H} CDR3 is capable of interacting with the residues of the V_{L}. In one embodiment, the substitutions in the Fc domain improve the pharmaceutical properties, optionally in vitro and/or in vivo stability of the antibody, optionally wherein the substitutions decrease the aggregation propensity of the antibody.

In one aspect, provided is an antibody comprising an Fc domain that is modified (compared to a wild-type Fc domain of the same isotype) to reduce binding between the Fc domain and human CD16A, CD16B, CD32A, CD32B and/or CD64 polypeptides, wherein the antibody comprises: (i) a heavy chain comprising CDR 1, 2 and 3 of the heavy chain variable region of SEQ ID NO: 6 and (ii) a light chain comprising CDR 1, 2 and 3 of the light chain variable region of SEQ ID NO: 7. In one aspect, the Fc domain is modified (compared to a wild-type Fc domain of the same isotype) to reduce binding between the Fc domain and human C1q polypeptide. In one embodiment, the antibody comprises an amino acid substitution in a heavy chain constant region at any one, two, three, four, five or more of residues selected from the group consisting of: 220, 226, 229, 233, 234, 235, 236, 237, 238, 243, 264, 268, 297, 298, 299, 309, 310, 318, 320, 322, 327, 330 and 331 (Kabat EU numbering). In one embodiment, the antibody has an amino acid substitution in a heavy chain constant region at any three, four, five or more of residues (Kabat numbering) selected from the group consisting of: 234, 235, 237, 322, 330 and 331.

In one aspect, provided is an antibody comprising an Fc domain that is modified (compared to a wild-type Fc domain of the same isotype) to reduce binding between the Fc domain and human CD16A, CD16B, CD32A, CD32B and/or CD64 polypeptides, wherein the antibody comprises: (i) a heavy chain comprising CDR 1, 2 and 3 of the heavy chain variable region of SEQ ID NO: 8 and (ii) a light chain comprising CDR 1, 2 and 3 of the light chain variable region of SEQ ID NO: 9. In one aspect, the Fc domain is modified (compared to a wild-type Fc domain of the same isotype) to reduce binding between the Fc domain and human C1q polypeptide. In one embodiment, the antibody comprises an amino acid substitution in a heavy chain constant region at any one, two, three, four, five or more of residues selected from the group consisting of: 220, 226, 229, 233, 234, 235, 236, 237, 238, 243, 264, 268, 297, 298, 299, 309, 310, 318, 320, 322, 327, 330 and 331 (Kabat EU numbering). In one embodiment, the antibody has an amino acid substitution in a heavy chain constant region at any three, four, five or more of residues selected from the group consisting of: 234, 235, 237, 322, 330 and 331.

In one aspect, provided is an anti-CD39 antibody capable of specifically inhibiting the enzymatic activity of membrane-bound CD39 protein (vascular CD39; the polypeptide of SEQ ID NO: 1) expressed at the surface of cells without substantially binding to human CD16 (and/or other Fcγ receptors) and/or C1q, and/or without substantially directing ADCC and/or CDC toward a CD39-expressing cell.

In one aspect, provided is an anti-CD39 antibody capable of inhibiting the enzymatic activity of membrane-bound vascular CD39 protein (comprising an amino acid sequence of SEQ ID NO: 1) expressed at the surface of cells without substantially causing the down-modulation (e.g. internalization) of cell surface-expressed CD39. In one embodiment, the antibodies do not substantially bind (e.g. via their Fc domain) to human Fcγ receptors (e.g. CD16, CD32a, CD32b, CD64) and/or C1q, and/or do not substantially directing ADCC and/or CDC toward a CD39-expressing cell. Optionally, the antibodies retain an Fc domain and retain binding to human FcRn.

In one aspect, the CD39-binding agent has decreased binding or substantially lacks binding to one or more soluble isoforms of human CD39 (e.g., isoforms L2 and/or L4). In one aspect, the CD39-binding agent has decreased binding or substantially lacks binding to one or more (or all of) isoforms L1, L2, L3 and L4 of human CD39.

In one embodiment, the antibodies are administered in an amount effective to neutralize the enzymatic activity of CD39 for a desired period of time, e.g. 1 week, 2 weeks, a month, until the next successive administration of anti-CD39 antibody.

In one embodiment, the antibodies are administered at a dosage and/or frequency that provides a blood concentration of antibody equal to at least the EC₅₀, EC₇₀ or EC₁₀₀ for inhibition of ATPase activity, optionally wherein the concentration is maintained for at least 1 week, 2 weeks, a month, or until the next successive administration of the anti-CD39 antibody. In one embodiment, the blood concentration is greater than the respective EC₅₀, EC₇₀ or EC₁₀₀ for ADCC activity towards CD39-expressing cells by an equivalent antibody that has an Fc domain that mediates CD16 binding, e.g. IgG1 (e.g. tumor cells, TReg cells and/or B cells).

In one aspect, provided are neutralizing anti-CD39 antibodies that do not cause substantial intracellular internalization of, or more generally down-modulation of, cell surface-expressed CD39 and/or do not depend thereupon for their CD39 inhibitory activity.

The disclosure in one aspect provides antibodies that bind an epitope present on human CD39 polypeptide expressed at the surface of cells, including but limited to tumor cells, and that inhibit the enzymatic (ATPase) activity of the CD39 enzyme without substantially causing the intracellular internalization of, or more generally down-modulation of, cell surface-expressed CD39 and/or do not depend thereupon for their CD39 inhibitory activity.

In one aspect, provided is an anti-CD39 antibody that binds an epitope on CD39 comprising an amino acid residue (e.g. one, two, three or four of the residues) selected from the group consisting of Q96, N99, E143 and R147 (with reference to SEQ ID NO: 1).

In one aspect, provided is an anti-CD39 antibody that has reduced binding to a CD39 polypeptide having a mutation at one, two, three or four of the residues selected from the group consisting of: Q96, N99, E143 and R147 (with reference to SEQ ID NO: 1); optionally, the mutant CD39 polypeptide has the mutations: Q96A, N99A, E143A and R147E.

In one embodiment, the CD39 neutralizing antibodies can be characterized by being capable of causing a decrease in cells' ATPase activity of CD39, optionally causing a decrease of AMP generation by a CD39-expressing cell, by at least 70%, 80% or 90%. In one embodiment, the CD39-neutralizing antibodies can be characterized by an E050 for inhibition of ATPase activity (e.g., EC₅₀ for inhibition of AMP generation by a CD39-expressing cell) of CD39 expressed by a cell of no more than 1 μg/ml, optionally no more than 0.5 μg/ml, optionally no more than 0.2 μg/ml.

Optionally, inhibition of ATPase activity of CD39 expressed by a cell is determined by assessing neutralization of ATPase activity in Ramos cells by quantifying AMP generated by hydrolysis of ATP (see, e.g., Example 6).

In one aspect, neutralization of the ATPase activity is determined by bringing CD39-expressing cells (e.g. Ramos lymphoma cells as used herein, available for example from the ATCC, reference CRL-1596) into contact with an antibody, and assessing production of AMP, e.g. by mass spectrometry, wherein a decrease in AMP generated indicates neutralization of ATPase activity. Optionally an antibody causes a decrease of AMP generated by at least 70%, 80% or 90% in this assay. Optionally an antibody causes a decrease of extracellular ATPase activity by a B cell of at least 70%, 80% or 90%.

In one aspect, provided is a neutralizing anti-CD39 antibody that binds an antigenic determinant present on CD39 expressed at the cell surface but lacking on membrane bound CD39 isoforms L1 and L3.

Provided in one aspect provided is a neutralizing anti-CD39 antibody that competes for binding to an epitope on CD39 bound by I-391, (e.g., that competes for binding to an epitope on a CD39 polypeptide with an antibody having the heavy and light chain CDRs or variable regions of any of I-391).

In one aspect of any of the embodiments herein, provided is an antigen-binding compound that binds the same epitope and/or competes for binding to a CD39 polypeptide with monoclonal antibodies I-391 (e.g., that competes for binding to a CD39 polypeptide with an antibody having the heavy and light chain CDRs or variable regions of I-391. In one embodiment, provided is antigen-binding compound binds the same epitope and/or competes for binding to a CD39 polypeptide with an antibody having respectively a V_{H} and V_{L} region of SEQ ID NOS: 6 and 7.

In one embodiment, an anti-CD39 antibody binds an epitope comprising one, two or three amino acid residues selected from the group consisting of the amino acid residues on CD39 bound by I-391.

In one aspect of any of the embodiments herein, the antibody may comprise a heavy chain comprising the three CDRs of the heavy chain variable region (VH) of antibody I-391 and a light chain comprising the three CDRs of the light chain variable region (VL) of antibody I-391.

In one aspect of any of the embodiments herein, the antibody may comprise a heavy chain comprising the three CDRs of the heavy chain variable region (VH) of antibody I-392 and a light chain comprising the three CDRs of the light chain variable region (VL) of antibody I-392.

In any of the embodiments herein, the anti-CD39 antibodies can be characterized by binding to human CD39 polypeptides expressed on the surface of a cell (e.g. a tumor cell, a cell made to express CD39, e.g. an Ramos tumor cell line, or a recombinant host cell made to express CD39, as shown in the Examples), and optionally further wherein the antibody binds with high affinity as determined by flow cytometry. For example, an antibody can be characterized by an EC₅₀, as determined by flow cytometry, of no more than 1 μg/ml, no more than 0.5 μg/ml, no more than 0.1 μg/ml or no more than 0.05 μg/ml, for binding to cells that express at their surface a CD39 polypeptide, e.g. tumor cells expressing CD39, cells expressing at their surface a CD39 polypeptide, lymphocytes expressing CD39, etc. Optionally, an antigen-binding compound has an EC₅₀ of no more than 1 μg/ml, optionally no more than 0.5 μg/ml, no more than 0.1 μg/ml, or no more than 0.05 μg/ml for binding to (i) cells expressing at their surface human CD39 (e.g. a polypeptide having the amino acid sequence of SEQ ID NO: 1) and/or (ii) cells expressing at their surface human non-human primate CD39 (e.g. a cynomolgus monkey CD39).

In one aspect of any of the embodiments herein, the anti-CD39 antibody is a tetrameric antibody comprising two heavy and two light chains, the heavy chains comprising Fc regions of human isotype and which substantially lack binding to human Fcγ receptors (e.g. CD16A, CD16B, CD32A, CD32B and/or CD64), and optionally further which substantially lack binding to human C1q polypeptides.

In one embodiment, the antibodies are administered to an individual having a cancer in an amount and frequency sufficient to neutralize the activity of CD39 in the tumor microenvironment. In one embodiment, the antibodies are administered in an amount and frequency sufficient to decrease the generation and/or concentration of adenosine in the tumor microenvironment. In one embodiment, the antibodies are administered in an amount and frequency sufficient to decrease the generation and/or concentration of AMP and/or adenosine in the tumor microenvironment. In one embodiment, the antibodies are administered in an amount and frequency sufficient to neutralize the activity of CD39 expressed by tumor cells. In one embodiment, the antibodies are administered in an amount and frequency sufficient to neutralize the activity of CD39 expressed by leukocytes or lymphocytes, e.g. CD4 T cells, CD8 T cells, TReg cells and/or B cells.

The antibodies will be useful in inhibiting CD39-mediated ATP hydrolysis, e.g. thereby leading to a decrease in the concentration of adenosine in the tumor microenvironment. These antibodies will therefore be useful in reversing the immunosuppressive effect of CD39 and/or adenosine on T cells, B cells and other cells that express adenosine receptors (A2A receptors), for example in the treatment of cancer. In one embodiment, the anti-CD39 antibody neutralizes adenosine-mediated inhibition of proliferation, cytokine production, cytotoxicity and/or NFκB activity in T cells.

The antibodies will be useful in inhibiting the production, amounts and/or concentrations of adenosine into the tumor microenvironment.

In another aspect provided is a method for treating an individual, the method comprising administering to an individual (e.g. an individual having a disease, a tumor, etc.) a therapeutically active amount of any of the anti-CD39 antigen binding compounds described herein. In one aspect provided is a method for treating an individual, the method comprising, consisting essentially of or consisting of: administering to an individual (e.g. an individual having a disease, a tumor, etc.) a therapeutically active amount of an antigen binding compound of the disclosure that inhibits a CD39 polypeptide. In one embodiment, the anti-CD39 antigen binding compound (e.g. antibody) is administered to an individual in combination with a second therapeutic agent, optionally a therapeutic agent (e.g. antibody) that neutralizes the inhibitory activity of human PD-1, optionally an anti-PD-1 antibody, optionally an anti-PD-L1 antibody. In one embodiment, the anti-CD39 antigen binding compound (e.g. antibody) is administered to an individual having a cancer and who has a poor response, or prognostic for response, to treatment with an agent that neutralizes the inhibitory activity of human PD-1. In one embodiment, the antibody inhibits a CD39 polypeptide in a cellular assay. The compound is in one embodiment a non-depleting antibody (an antibody that does not deplete cells to which it binds, e.g., an Fc silent antibody). Optionally, the compound binds to CD39 polypeptides in bivalent manner. Optionally, the antibody is a chimeric, humanized or human antibody. Optionally, the antibody comprises a heavy chain constant region of IgG (e.g. IgG1) isotype modified to eliminate binding to human Fcγ receptors (e.g. CD16A, CD16B, CD32A, CD32B and/or CD64).

In another aspect, antibodies having increased stability and/or solubility in conventional pharmaceutical formulations can advantageously be combined in pharmaceutical formulations with other antibodies. Provided in one embodiment is a pharmaceutical formulation comprising (i) an antibody that inhibits a CD39 polypeptide and displays increased stability, e.g. an antibody that inhibits a CD39 polypeptide comprising a plurality of aromatic resides in a CDR and a modified human IgG1 Fc domain comprising an amino acid substitution at any three, four, five or more of residues at Kabat positions 234, 235, 237, 322, 330 and 331, and (ii) a second antibody of human IgG isotype, optionally wherein the second antibody has anti-cancer activity. In one embodiment, the second antibody is capable of inducing ADCC toward a cell to which it is bound, optionally the second antibody binds to an antigen present on a tumor cell (a tumor antigen). In one embodiment, the second antibody is capable of a neutralizing the activity of a protein to which its hypervariable region binds. Provided in one embodiment is a pharmaceutical formulation comprising (i) an antibody that inhibits a CD39 polypeptide and displays increased stability, e.g. an antibody that inhibits a CD39 polypeptide comprising a plurality of aromatic resides in a CDR and a modified human IgG1 Fc domain comprising an amino acid substitution at any three, four, five or more of residues at Kabat positions 234, 235, 237, 322, 330 and 331, and (ii) a second antibody of human IgG isotype, wherein the second antibody neutralizes the inhibitory activity of human PD-1, optionally an anti-PD-1 antibody, optionally an anti-PD-L1 antibody. In one embodiment, both the anti-CD39 antibody and the second antibody comprise a modified human IgG1 Fc domain comprising an amino acid substitution at any three, four, five or more of residues at Kabat positions 234, 235, 237, 322, 330 and 331.

In one aspect provided is a method for decreasing ATP hydrolysis by a CD39-expressing cell (e.g. a leukocyte and/or a tumor cell in an individual), or a method for neutralizing of the enzymatic activity of cellular CD39, the method comprising, consisting essentially of or consisting of: bringing the CD39-expressing cell into contact with an antibody of the disclosure that inhibits CD39. In one embodiment, the step of bringing the CD39-expressing cell into contact with an antigen binding compound of the disclosure comprises administering to an individual a therapeutically active amount of an antibody that inhibits CD39. In one embodiment the individual has a cancer.

In one aspect provided is a method for decreasing adenosine present in the tumor environment (e.g. in an individual), the method comprising, consisting essentially of or consisting of: administering to an individual a therapeutically active amount of an antibody of the disclosure that inhibits a CD39 polypeptide. In one embodiment the individual has a cancer.

In one embodiment, the active amount of an antibody that inhibits a CD39 polypeptide is an amount effective to achieve and/or maintain (e.g. until the subsequent administration of antigen binding compound) a blood concentration of at least the EC₅₀, optionally the EC₇₀, optionally substantially the EC₁₀₀, for inhibition of CD39-mediated catabolism of ATP to AMP in an individual. In one embodiment, the active amount of an antigen binding compound that inhibits a CD39 polypeptide is an amount effective to achieve the EC₅₀, optionally the EC₇₀, optionally substantially the EC₁₀₀, for inhibition of CD39-mediated catabolism of ATP to AMP in an extravascular tissue of an individual. In one embodiment, the active amount an antigen binding compound that inhibits a CD39 polypeptide is an amount effective to achieve the EC₅₀, optionally the EC₇₀, optionally substantially the EC₁₀₀, for inhibition of CD39-mediated catabolism of ATP to AMP in an individual. In one embodiment, the active amount of an antigen binding compound that inhibits a CD39 polypeptide is between 1 and 20 mg/kg body weight. In one embodiment, the active amount is administered to an individual weekly, every two weeks, monthly or every two months.

Optionally the individual is human having or who is susceptible to having a cancer. Optionally the individual is human having or who is susceptible to having a cancer characterized by malignant cells that express vascular CD39. Optionally the individual is human having or who is susceptible to having a cancer and who has detectable levels of circulating or tumor-infiltrating leukocytes that express vascular CD39. Optionally, the individual treated with a vascular CD39-specific antibody of the disclosure has detectable levels of a soluble CD39 isoform, e.g. isoforms L2 and/or L4 (e.g. the isoform is at detectable levels in circulation or in an extravascular tissue).

The antibodies are optionally characterized by binding affinity (K_{D}) for a human CD39 polypeptide of less than (better than) 10⁻⁹ M, preferably less than 10⁻¹⁰ M, or preferably less than 10⁻¹¹M, and/or by binding human CD39 with an EC₅₀ lower than (better binding than) 1 μg/ml, preferably wherein the antibody has an EC₅₀ of no more than 0.5 μg/ml, optionally no more than 0.2 μg/ml, optionally no more than 0.1 μg/ml, for binding to cells (e.g. tumor cells) expressing human CD39 at the cell surface.

The antibodies are optionally chimeric, human or humanized antibodies.

The antibodies are optionally characterized by an EC₅₀ for neutralization of the enzymatic activity of CD39 in CD39-expressing cells of less than (better than) 1 μg/ml, optionally less than 0.5 μg/ml.

In one embodiment, the antibody is a monoclonal antibody or a fragment thereof that retains binding specificity and ability to neutralize the enzymatic activity of CD39. In one embodiment, the antibody is an IgG1 antibody. For example, the antibody may be an antibody comprising an Fc domain of human IgG1 isotype modified to reduce binding between the Fc domain and an Fcγ receptor (e.g. CD16). In one embodiment, the antigen-binding compound does not comprise a Fc domain capable of inducing antibody mediated cellular cytotoxicity (ADCC) and/or CDC; optionally the antigen-binding compound does not comprise an Fc domain capable of substantially binding to a FcγRIIIA (CD16) polypeptide (e.g., comprises an Fc domain not capable of substantially binding to a FcγRIIIA (CD16) polypeptide; lacks an Fc domain (e.g. lacks a CH2 and/or CH3 domain; comprises an Fc domain of IgG4 isotype). In one embodiment, the Fc domain (e.g. of human IgG1, IgG2, IgG3 or IgG4 isotype) comprises an amino acid modification (e.g. substitution) compared to a wild-type Fc domain, wherein the substitution reduces the ability of the Fc domain (or antibodies containing it) to bind to an Fcγ receptor (e.g. CD16) and/or to bind complement. Optionally, the substitution increases or ameliorates the in vivo and/or in vitro stability (e.g. decreases aggregation propensity) of an antibody comprising a CDR (e.g. V_{H} CDR3) comprising a plurality (e.g., 3, 4, 5, 6 or more) of aromatic amino acid residues, optionally tyrosines and/or phenylalanines. In one embodiment, the antigen-binding compound is not linked to a toxic moiety.

Also provided are nucleic acids encoding the human or humanized antibody or antibody fragment having any of the foregoing properties, a vector comprising such a nucleic acid, a cell comprising such a vector, and a method of producing a human anti-CD39 antibody, comprising culturing such a cell under conditions suitable for expression of the anti-CD39 antibody. The disclosure also relates to compositions, such as pharmaceutically acceptable compositions and kits, comprising such proteins, nucleic acids, vectors, and/or cells and typically one or more additional ingredients that can be active ingredients or inactive ingredients that promote formulation, delivery, stability, or other characteristics of the composition (e.g., various carriers). The disclosure further relates various new and useful methods making and using such antibodies, nucleic acids, vectors, cells, organisms, and/or compositions, such as in the modulation of CD39-mediated biological activities, for example in the treatment of diseases related thereto, notably cancers.

The disclosure also provides a method of potentiating the activity of lymphocytes (e.g., T cells) in a subject in need thereof, or for restoring the activity of lymphocytes (e.g., T cells), or a method of relieving the adenosine-mediated inhibition of lymphocytes (e.g., T cells), which method comprises administering to the subject an effective amount of any of the foregoing compositions. In one embodiment, the subject is a patient suffering from cancer. For example, the patient may be suffering from a solid tumor, e.g. colorectal cancer, renal cancer, ovarian cancer, lung cancer, breast cancer or malignant melanoma. Alternatively, the patient may be suffering from a hematopoietic cancer, e.g., acute myeloid leukaemia, chronic myeloid leukaemia, multiple myeloma, or non-Hodgkin's lymphoma.

The disclosure also provides a method for treatment of disease in an individual, the treatment comprising administering to the individual an anti-CD39 antibody that neutralizes the enzymatic activity of CD39 for at least one administration cycle in which the anti-CD39 antibody is administered at least once, optionally at least twice, in an amount effective to achieve, and/or to maintain between two successive administrations of the anti-CD39 antibody, a concentration in blood (serum) or an extravascular tissue (e.g. tumor environment) that corresponds to at least the EC₅₀ (e.g. an EC₅₀ between 0.01 and 0.5 μg/ml), optionally the EC₇₀ or optionally the EC₁₀₀, for neutralization of the enzymatic activity of CD39 (e.g. an EC₁₀₀ between 0.05 and 1 μg/ml, between 0.1 and 1 μg/ml). The antibody can for example be administered in an amount to achieve and/or maintained a concentration in circulation or in an extravascular tissue (e.g. tumor environment) of at least about 0.1 μg/ml, 0.5 μg/ml, 1 μg/ml or 2 μg/ml). For example, to achieve a concentration in an extravascular tissue of between 0.05 and 1 μg/ml, or between 0.1 and 1 μg/ml, the anti-CD39 antibody is administered in amounts effective to achieve a concentration in circulation of the anti-CD39 antibody of between 0.5 and 10 μg/ml, or between 1 and 10 μg/ml. Optionally, the anti-CD39 antibody is administered at least twice and in amounts effective to maintain the concentration of the anti-CD39 antibody at least the aforementioned concentration for at least 1 week, 2 weeks, 3 weeks, 4 weeks, between two successive administrations of the anti-CD39 antibody and/or throughout the administration cycle.

The disclosure also provides a method for treatment of disease in an individual, the treatment comprising administering to the individual an anti-CD39 antibody that neutralizes the enzymatic activity of CD39 for at least one administration cycle in which the anti-CD39 antibody is administered at least once, optionally at least twice, in an amount effective to achieve, and/or to maintain between two successive administrations of the anti-CD39 antibody, a blood or tissue concentration of anti-CD39 antibody of at least 1 μg/ml, optionally at least 10 μg/ml, optionally between 1 and 100 μg/ml. Optionally, the anti-CD39 antibody is administered at least twice and in amounts effective to maintain a continuous blood or tissue concentration of the anti-CD39 antibody of at least 1 μg/ml, optionally at least 10 μg/ml, optionally between 1 and 100 μg/ml, for at least 1 week, 2 weeks, 3 weeks, 4 weeks, between two successive administrations of the anti-CD39 antibody and/or throughout the administration cycle.

These aspects are more fully described in, and additional aspects, features, and advantages will be apparent from, the description provided herein.

### BRIEF DESCRIPTION OF THE DRAWINGS

FIG. 1 shows titration by ELISA for binding to recombinant human and cynomolgus CD39.

FIG. 2 shows titration by ELISA for binding by I-391 antibody to recombinant human CD39 isoforms: vascular CD39, CD39-L1, CD39-L2, CD39-L3 and CD39-L4. Antibody I-391 bound only vascular CD39, without any binding to -L1, CD39-L2, CD39-L3 or CD39-L4. Isotype control (IC) of HUS2 or mouse IgG2a format antibodies do not bind any CD39 or CD39-L molecules. The top panel shows antibody I-391 or isotype control having a human IgG1 Fc domain mutated to lose binding to human Fcγ receptors (HUS2); the bottom panel shows antibodies with Fc domain of mouse IgGa isotype (MOGA).

FIG. 3 shows that following incubation with I-391, CD39 expression remained stable and comparable to incubation in the absence of Ab, and no decrease in bound I-391 could be detected, indicated that I-391 did not induce CD39 down modulation nor CD39 internalization. CD39 expression is assessed using the A1 antibody which does not compete for binding to CD39 with I-391.

FIGS. 4 and 5 show results from a study of anti-CD39/CD39 complexes by X-ray diffraction. The 3-dimensional structure is illustrated, showing that binding of the neutralizing anti-CD39 to the target antigen CD39 entirely relies on the heavy chain variable domain; the anti-CD39 antibody light chain does not contact the antigen directly.

FIG. 6 shows results from a study of anti-CD39/CD39 complexes by X-ray diffraction. The anti-CD39 heavy chain binds to both the CD39 N-terminal domain 1 and C-terminal domain 2 of CD39). The anti-CD39 binding site is located at the apex of the two CD39 domains and at the entry of the catalytic cleft.

FIG. 7 shows results from a study of anti-CD39/CD39 complexes by X-ray diffraction. The human CD39/anti-CD39 frozen conformation perfectly superimposes with rat CD39 form A of the pdb crystal 3ZX3. Binding of the antibody to both domains at the same time thus likely inhibits domain motion and block the enzyme in a given frozen status.

FIG. 8 shows several human IgG1 Fc domain mutants showed a higher aggregation temperature (TAgg) and improved stability of the antibody compared to wild-type human Fc domains.

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of” or by “consisting of”.

Human CD39, also known as “vascular” CD39, NTPdase1, ENTPD1, ATPDase and vascular ATP diphosphohydrolase, exhibits ATPase activity. CD39 hydrolyzes extracellular ATP and ADP to AMP, which is further converted to adenosine by another enzyme, 5-prime nucleotidase. The amino acid sequence of the “vascular” human CD39 mature polypeptide chain is shown in Genbank under accession number P49961, the entire disclosure of which is incorporated herein by reference, and as follows:

| (SEQ ID NO: 1) | |
|---|---|
| 1 | MEDTKESNVK TFCSKNILAI LGFSSIIAVI ALLAVGLTQN KALPENVKYG IVLDAGSSHT |
| 61 | SLYIYKWPAE KENDTGVVHQ VEECRVKGPG ISKFVQKVNE IGIYLTDCME RAREVIPRSQ |
| 121 | HQETPVYLGA TAGMRLLRME SEELADRVLD VVERSLSNYP FDFQGARIIT GQEEGAYGWI |
| 181 | TINYLLGKFS QKTRWFSIVP YETNNQETFG ALDLGGASTQ VTFVPQNQTI ESPDNALQFR |
| 241 | LYGKDYNVYT HSFLCYGKDQ ALWQKLAKDI QVASNEILRD PCFHPGYKKV VNVSDLYKTP |
| 301 | CTKRFEMTLP FQQFEIQGIG NYQQCHQSIL ELFNTSYCPY SQCAFNGIFL PPLQGDFGAF |
| 361 | SAFYFVMKFL NLTSEKVSQE KVTEMMKKFC AQPWEEIKTS YAGVKEKYLS EYCFSGTYIL |
| 421 | SLLLQGYHFT ADSWEHIHFI GKIQGSDAGW TLGYMLNLTN MIPAEQPLST PLSHSTYVFL |
| 481 | MVLFSLVLFT VAIIGLLIFH KPSYFWKDMV |

Human CD39-L1, also known as NTPDase2 or ENTPD2, is shown in Genbank under accession number NP_001237, the entire disclosure of which is incorporated herein by reference, and as follows:

| (SEQ ID NO: 2) | |
|---|---|
| 1 | MAGKVRSLLP PLLLAAAGLA GLLLLCVPTR DVREPPALKY GIVLDAGSSH TSMFIYKWPA |
| 61 | DKENDTGIVG QHSSCDVPGG GISSYADNPS GASQSLVGCL EQALQDVPKE RHAGTPLYLG |
| 121 | ATAGMRLLNL TNPEASTSVL MAVTHTLTQY PFDFRGARIL SGQEEGVFGW VTANYLLENF |
| 181 | IKYGWVGRWF RPRKGTLGAM DLGGASTQIT FETTSPAEDR ASEVQLHLYG QHYRVYTHSF |
| 241 | LCYGRDQVLQ RLLASALQTH GFHPCWPRGF STQVLLGDVY QSPCTMAQRP QNFNSSARVS |
| 301 | LSGSSDPHLC RDLVSGLFSF SSCPFSRCSF NGVFQPPVAG NFVAFSAFFY TVDFLRTSMG |
| 361 | LPVATLQQLE AAAVNVCNQT WAQQLLSRGY GFDERAFGGV IFQKKAADTA VGWALGYMLN |
| 421 | LTNLIPADPP GLRKGTDFSS WVVLLLLFAS ALLAALVLLL RQVHSAKLPS TI |

Human CD39-L2, also known as NTPDase6 or ENTPD6; ENTPD6 isoform 1 is shown in Genbank under accession number NP_001238, the entire disclosure of which is incorporated herein by reference, and as follows:

| (SEQ ID NO: 3) | |
|---|---|
| 1 | MKKGIRYETS RKTSYIFQQP QHGPWQTRMR KISNHGSLRV AKVAYPLGLC VGVFIYVAYI |
| 61 | KWHRATATQA FFSITRAAPG ARWGQQAHSP LGTAADGHEV FYGIMFDAGS TGTRVHVFQF |
| 121 | TRPPRETPTL THETFKALKP GLSAYADDVE KSAQGIRELL DVAKQDIPFD FWKATPLVLK |
| 181 | ATAGLRLLPG EKAQKLLQKV KEVFKASPFL VGDDCVSIMN GTDEGVSAWI TINFLTGSLK |
| 241 | TPGGSSVGML DLGGGSTQIA FLPRVEGTLQ ASPPGYLTAL RMFNRTYKLY SYSYLGLGLM |
| 301 | SARLAILGGV EGQPAKDGKE LVSPCLSPSF KGEWEHAEVT YRVSGQKAAA SLHELCAARV |
| 361 | SEVLQNRVHR TEEVKHVDFY AFSYYYDLAA GVGLIDAEKG GSLVVGDFEI AAKYVCRTLE |
| 421 | TQPQSSPFSC MDLTYVSLLL QEFGFPRSKV LKLTRKIDNV ETSWALGAIF HYIDSLNRQK |
| 481 | SPAS |

Human CD39-L3, also known as NTPDase3 or ENTPD3; ENTPD3 isoform is shown in Genbank under accession number NP_001239, the entire disclosure of which is incorporated herein by reference, and as follows:

| (SEQ ID NO: 4) | |
|---|---|
| 1 | MFTVLTRQPC EQAGLKALYR TPTIIALVVL LVSIVVLVSI TVIQIHKQEV LPPGLKYGIV |
| 61 | LDAGSSRTTV YVYQWPAEKE NNTGVVSQTF KCSVKGSGIS SYGNNPQDVP RAFEECMQKV |
| 121 | KGQVPSHLHG STPIHLGATA GMRLLRLQNE TAANEVLESI QSYFKSQPFD FRGAQIISGQ |
| 181 | EEGVYGWITA NYLMGNFLEK NLWHMWVHPH GVETTGALDL GGASTQISFV AGEKMDLNTS |
| 241 | DIMQVSLYGY VYTLYTHSFQ CYGRNEAEKK FLAMLLQNSP TKNHLTNPCY PRDYSISFTM |
| 301 | GHVFDSLCTV DQRPESYNPN DVITFEGTGD PSLCKEKVAS IFDFKACHDQ ETCSFDGVYQ |
| 361 | PKIKGPFVAF AGFYYTASAL NLSGSFSLDT FNSSTWNFCS QNWSQLPLLL PKFDEVYARS |
| 421 | YCFSANYIYH LFVNGYKFTE ETWPQIHFEK EVGNSSIAWS LGYMLSLTNQ IPAESPLIRL |
| 481 | PIEPPVFVGT LAFFTAAALL CLAFLAYLCS ATRRKRHSEH AFDHAVDSD |

Human CD39-L4, also known as NTPDase5 or ENTPD5, is shown in Genbank under accession number NP_001240 (precursor), the entire disclosure of which is incorporated herein by reference, and as follows:

| (SEQ ID NO: 5) | |
|---|---|
| 1 | MATSWGTVFF MLVVSCVCSA VSHRNQQTWF EGIFLSSMCP INVSASTLYG IMFDAGSTGT |
| 61 | RIHVYTFVQK MPGQLPILEG EVFDSVKPGL SAFVDQPKQG AETVQGLLEV AKDSIPRSHW |
| 121 | KKTPVVLKAT AGLRLLPEHK AKALLFEVKE IFRKSPFLVP KGSVSIMDGS DEGILAWVTV |
| 181 | NFLTGQLHGH RQETVGTLDL GGASTQITFL PQFEKTLEQT PRGYLTSFEM FNSTYKLYTH |
| 241 | SYLGFGLKAA RLATLGALET EGTDGHTFRS ACLPRWLEAE WIFGGVKYQY GGNQEGEVGF |
| 301 | EPCYAEVLRV VRGKLHQPEE VQRGSFYAFS YYYDRAVDTD MIDYEKGGIL KVEDFERKAR |
| 361 | EVCDNLENFT SGSPFLCMDL SYITALLKDG FGFADSTVLQ LTKKVNNIET GWALGATFHL |
| 421 | LQSLGISH |

In the context herein, “neutralize” or neutralizing” when referring to the CD39 polypeptide (e.g. “neutralize CD39”, “neutralize the activity of CD39” or “neutralize the enzymatic activity of CD39”), refers to a process in which the ATP hydrolysis (ATPase) activity of CD39 is inhibited. This comprises, notably the inhibition of CD39-mediated generation of AMP and/or ADP, i.e. the inhibition of CD39-mediated catabolism of ATP to AMP and/or ADP. This can be measured for example in a cellular assay that measures the capacity of a test compound to inhibit the conversion of ATP to AMP and/or ADP, either directly or indirectly. For example, disappearance of ATP and/or generation of AMP can be assessed, as described herein. In one embodiment, an antibody preparation causes at least a 60% decrease in the conversion of ATP to AMP, at least a 70% decrease in the conversion of ATP to AMP, or at least an 80% or 90% decrease in the conversion of ATP to AMP, referring, for example, to the assays described herein (e.g. disappearance of ATP and/or generation of AMP).

Whenever “treatment of cancer” or the like is mentioned with reference to anti-CD39 binding agent (e.g. antibody), this can include: (a) method of treatment of cancer, said method comprising the step of administering (for at least one treatment) an anti-CD39 binding agent, (preferably in a pharmaceutically acceptable carrier material) to an individual, a mammal, especially a human, in need of such treatment, in a dose that allows for the treatment of cancer, (a therapeutically effective amount), preferably in a dose (amount) as specified herein; (b) the use of an anti-CD39 binding agent for the treatment of cancer, or an anti-CD39 binding agent, for use in said treatment (especially in a human); (c) the use of an anti-CD39 binding agent for the manufacture of a pharmaceutical preparation for the treatment of cancer, a method of using an anti-CD39 binding agent for the manufacture of a pharmaceutical preparation for the treatment of cancer, comprising admixing an anti-CD39 binding agent with a pharmaceutically acceptable carrier, or a pharmaceutical preparation comprising an effective dose of an anti-CD39 binding agent that is appropriate for the treatment of cancer; or (d) any combination of a), b), and c), in accordance with the subject matter allowable for patenting in a country where this application is filed.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g. CD39, as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody, it means that the antibody competes with the monoclonal antibody in a binding assay using either recombinant CD39 molecules or surface expressed CD39 molecules. For example, if a test antibody reduces the binding of a reference antibody to a CD39 polypeptide or CD39-expressing cell in a binding assay, the antibody is said to “compete” respectively with the reference antibody.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “internalization”, used interchangeably with “intracellular internalization”, refers to the molecular, biochemical and cellular events associated with the process of translocating a molecule from the extracellular surface of a cell to the intracellular surface of a cell. The processes responsible for intracellular internalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “inducing and/or increasing internalization” comprises events wherein intracellular internalization is initiated and/or the rate and/or extent of intracellular internalization is increased.

The term “agent” is used herein to denote a chemical compound, a mixture of chemical compounds, a biological macromolecule, or an extract made from biological materials. The term “therapeutic agent” refers to an agent that has biological activity.

For the purposes herein, a “humanized” or “human” antibody refers to an antibody in which the constant and variable framework region of one or more human immunoglobulins is fused with the binding region, e.g. the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

### Production of Antibodies

The anti-CD39 antibody that can be used for the treatment of cancers and/or other diseases (e.g., infectious disease) binds an extra-cellular portion of human CD39 polypeptide and neutralizes the enzymatic activity of CD39 expressed on the surface of a cell, e.g. a tumor cell. In one embodiment the agent inhibits the ATPase activity of CD39. In one embodiment the antibody inhibits CD39-mediated generation of adenosine. In one embodiment the antibody inhibits CD39-mediated catabolism of ATP to AMP. In one embodiment the antibody inhibits adenosine-mediated inhibition of lymphocyte activity (e.g. T cells). In one aspect, the antibody is selected from a full-length antibody, an antibody fragment, and a synthetic or semi-synthetic antibody-derived molecule.

Antibodies that potently inhibit the enzymatic (ATPase activity) activity of the CD39 enzyme appear to do so by immobilizing the enzyme in one of its conformations thereby preventing it from hydrolyzing its substrate. The antibodies achieve this by binding to both C- and N-terminal domains of CD39 at the same time.

Thus in any embodiment an anti-CD39 antibody can bind to both C- and N-terminal domains of CD39 via their V_{H} CDRs (there is no need for the V_{L} CDRs to bind CD39 at all). As shown in the Examples, binding to the N-terminal domain (domain 1) of CD39 can be achieved by two of the CDRs (CDR1 and CDR3, as well as a first segment of Kabat CDR2, while binding to the C-terminal domain (domain 2) can be achieved through a single CDR (CDR2) (a CDR2 that extends into the Kabat FR3). Not only is the contribution of CDR2 surprising, but additionally the CDR2 cooperates with FR3 in binding to CD39. Furthermore, the binding by CDR2-FR to CD39 involves the glycan at N292 of CD39 (the N292 glycan covers a significant amount of the apical surface of the C-terminal domain). The resulting binding by the V_{H} to both domains 1 and 2 is believed to be important in immobilizing the CD39 enzyme so as to prevent substrate hydrolysis. The antibodies thus have a V_{H} CDR2 that binds to the N-terminal domain of CD39 via a first portion of the CDR2 and to the C-terminal domain of CD39 (including the glycan at N292) via a second portion of the CDR2, in combination with FR3 residues.

The inventors further observed that the positioning of the V_{H} CDR3 for binding to CD39 occurs via a surprising mechanism involving a V_{L} CDR/V_{H} CDR3/CD39 binding matrix in which V_{H} CDR3 is trapped between the V_{L} CDRs and CD39. The V_{L} CDRs form a paratope that binds to the CDR3 of the V_{H}. The V_{H} CDR3 comprises numerous aromatic residues, and while one of the aromatic residues in V_{H} CDR3 interacts with CD39, other aromatic amino acids within V_{H} CDR3 interact with the residues of the V_{L}. The V_{H} CDR3 comprises multiple aromatic residues which form pi-interactions with the V_{L} on one face, and CD39 on another face, resulting in a matrix of pi-interactions structure that traps the V_{H} CDR3 between the V_{L} CDRs and CD39 and permits a large binding area across the surface of CD39. Further surprisingly, the structure appears to have arisen in the context of certain framework residues, as both the V_{H} CD39 interaction and the V_{H} CDR3-V_{L} interaction involve framework residues (according to Kabat numbering), such that the overall contact residues involved a limited set of Kabat CDR residues combined with Kabat FR residues.

The overall structure permits the V_{H} to bind N-terminal domain of CD39 (e.g., at residues V95, Q96, K97, L137, E140, L144 of CD39, with reference to the CD39 amino acid sequence of SEQ ID NO: 1) on one side of the enzymatic active site, and the C-terminal domain of CD39 (e.g., at residues S294, K298, as well as to the glycan linked to N292 of CD39, of CD39) on the opposite side of the enzymatic active site.

These findings permit a wide range of CD39-binding polypeptides to be generated that retain the mechanism of action and permits desired amino acid sequences and features to be incorporated. Antibodies and other V_{H}/V_{L}-containing proteins can be designed which include, inter alia, the V_{H} CDR2 segment that binds the N-terminal domain, the V_{H} CDR2-FR3 segment capable of binding the C-terminal domain (and, notably, the N292-linked glycosylation) and the V_{H} CDR3 that binds the C-terminal domain and gives rise to the V_{L}CDR—V_{H}CDR3-CD39 matrix. Amino acid sequences of V_{L} domains can be selected in the context of the desired V_{H} sequence, e.g., by introducing substitutions that maintain the overall V_{H} or V_{L} domain structure without affecting V domain interactions, and where V domain interactions would be modified, making amino acid substitutions pair-wise in both V_{H} and V_{L} at the respective contact positions. The resulting protein binds to CD39 essentially or exclusively via the V_{H} domain(s), and the V_{L} domain(s) binds to V_{H} CDR3 but is not involved in binding to CD39. A wide range of both V_{L} CDR amino acid sequences can be employed in such protein; optionally V_{L} CDR residues and the respective V_{H} residues can be chosen and substituted as pairs such that the V_{H}/V_{L} CDR contact is maintained. Similarly, a wide range of human (or non-human) V_{H} and V_{L} framework sequences can be selected as acceptor frameworks that bear resides at the specified positions that maintain the V_{H} CDR-V_{L}CDR contact and the V_{H}CDR-CD39 contact.

Consequently, in one embodiment, an anti-CD39 antigen binding domain, or an antigen-binding protein that comprises the antigen binding domain (e.g., an antibody or antibody fragment, a multispecific binding protein, a bispecific antibody, etc.), comprises complementary determining regions (CDR) and framework regions (FR). The antigen binding domains can be designed or modified so as to provide desired and/or improved properties.

In one embodiment, an anti-CD39 antigen-binding protein is capable of binding to and inhibiting the activity of a human CD39 polypeptide, the antigen-binding protein comprising a VH and a VL that each comprise a framework (e.g. a framework having an amino acid sequence of human origin) and a CDR1, CDR2 and CDR3, wherein the antigen-binding protein is capable of binding to the N-terminal domain of CD39 and the C-terminal domain of CD39. In one embodiment, the antigen-binding protein restricts the domain movement of CD39 when bound to CD39. Optionally, the VH and/or VL framework (e.g. FR1, FR2, FR3 and/or FR4) is of human origin. In one embodiment, the V_{H} comprises a first CDR (or antigen binding domain) that is capable of binding to the N-terminal domain of CD39 and a second CDR (or antigen binding domain) that that is capable of binding to amino acid residues of the C-terminal domain of CD39.

In one aspect (e.g. in any aspect herein), a binding molecule or antigen-binding fragment thereof is capable of binding to and inhibiting the activity of CD39, comprising a V_{H} and a V_{L}, wherein the Kabat CDR1 of the V_{H} binds to amino acid residues in the N-terminal domain of CD39, the Kabat CDR2 (optionally together with the FR3) of the V_{H} binds to amino acid residues and/or to the N292-linked glycan in the C-terminal domain of CD39, and the Kabat CDR3 of the V_{H} binds to amino acid residues the N-terminal domain of CD39. Optionally the CDR2 of the V_{H} comprises a first amino acid segment that binds to the N-terminal domain of CD39 together and an amino acid segment that binds, together with FR3 residues, to the C-terminal domain of CD39. Optionally, the CDR3 comprises an aromatic residue (e.g. a tyrosine) that is capable of binding an amino acid residue in the N-terminal domain of CD39 and a second aromatic amino acid residue (e.g., a tyrosine, a phenylalanine) that is capable of contacting an amino acid residue in the V_{L}. Optionally, the binding molecule or antigen-binding fragment comprises a V_{L} that binds, via a residue in a Kabat CDR, to the Kabat CDR3 of the V_{H}.

In one embodiment, the CDR1 comprises a residue that is capable of contacting amino acid residues in the N-terminal domain of CD39. Optionally a CD39 contact residue is at Kabat position 33, optionally at both positions 31 and 33. In one embodiment, the Kabat FR1 comprise a CD39 contact residue at Kabat position 30, optionally wherein the residue is a threonine. Optionally the residue at position 31 is a histidine or asparagine. Optionally the residue at position 33 is a glycine. Optionally, the residue at position 32 is a residue with an aromatic side chain (an aromatic residue).

In one embodiment, the CDR2 and FR3 comprise a segment of residues within Kabat positions 59-71, optionally within 59-72b, that is capable of contacting amino acid residues in the C-terminal domain of CD39, optionally further wherein residues within Kabat positions 59-71 contact the glycan at residue N292 of CD39. For example, the Kabat CDR2 and FR3 can comprise residues at Kabat positions 59, 65, 67, 68, 69, 70 and/or 71, and optionally further at residue 72, 72a and/or 72b that are capable of contacting the C-terminal domain of CD39, e.g. including amino acid resides in CD39 and the glycan at N292 of the CD39 polypeptide.

In one embodiment, the CDR2 (e.g., Kabat CDR2-FR3 segment) comprises residues at Kabat position 59-71 having the formula X₁ X₂ X₃ X₄ X₅ X₆ X₇ X₈ X₉ X₁₀ X₁₁ X₁₂ X₁₃ (SEQ ID NO: 12), wherein X₁ represents a tyrosine, each of X₂, X₃, X₄, X₅ and X₆ each represent any amino acid, X₇ represents glycine or another residue which does not introduce steric hindrance that reduces antigen binding, X₈ represents any amino acid, X₉ represents phenylalanine or another hydrophobic residue capable of maintaining the beta-strand position and V_{H} domain structure integrity, X₁₀ represents alanine or valine, or optionally leucine, optionally threonine, optionally a hydrophobic residue, X₁₁ represents phenylalanine or another hydrophobic residue (e.g. isoleucine) capable of maintaining the beta-strand position and V_{H} domain structure integrity and X₁₂ represents serine, and X₁₃ represents any amino acid, optionally leucine, optionally alanine, valine, threonine or arginine. In one embodiment, the V_{H} FR3 residues at Kabat positions 72, 72a and 72b have the formula X₁ X₂ X₃, wherein X₁ represents aspartic acid, glutamic acid or alanine, X₂ represents any amino acid, optionally alanine, threonine or asparagine, or a conservative substitution thereof, and X₃ represents serine, optionally alanine, or a conservative substitution thereof.

In one embodiment, the V_{H} comprises a leucine residue at Kabat position 71 (FR3). Human or humanized antibodies can advantageously have the FR3 signature sequence FVFSL at Kabat positions 67-71, present in certain human V_{H} FR domains. Thus in one aspect of any embodiment herein, the segment of residues at Kabat positions 67-71 comprises the amino acid sequence: FVFSL.

In one embodiment, the CDR2 optionally comprises a segment of residues in Kabat positions 50-56, optionally residues 50, 52, 52a, 53 and/or 56, that is capable of contacting amino acid residues in CD39 (e.g. in the N-terminal domain of CD39), optionally wherein the residue at position 53 aromatic is an amino acid residue. In one embodiment, the CDR2 comprises residues 50, 52, 52a, 53 and/or 56, that are capable of contacting amino acid residues in CD39. In one embodiment, the CDR2 comprises residues at Kabat position 50-56 having the formula X₁ X₂ X₃ X₄ X₅ X₆ X₇ X₈ (SEQ ID NO: 13), wherein X₁ represents tryptophan, X₂ represents any amino acid, optionally an isoleucine, X₃ represents asparagine or optionally glutamine, X₄ represents threonine, X₅ represents any amino acid residue, optionally a tyrosine or optionally phenylalanine, X₆ represents any amino acid, optionally threonine, optionally serine, optionally asparagine, alanine or glycine, optionally a residue other than a large or hydrophobic residue, X₇ represents any amino acid, optionally glycine, optionally alanine, serine, threonine, asparagine or glutamine, optionally a residue other than aspartic acid or glutamic acid, optionally a residue other than lysine or arginine, X₈ represents glutamic acid, optionally aspartic acid.

In another embodiment, a V_{H} comprises a Kabat CDR2-FR3 segment that binds to both the N- and C-terminal domain of CD39 (e.g., across the N- and C-domain surface, across the substrate cleft, groove entry or active site) the CDR2-FR3 segment comprising residues (e.g. at Kabat position 50-71) having the formula X₁ X₂ X₃ X₄ X₅ X₆ X₇ X₈ X₉ X₁₀ X₁₁ X₁₂ X₁₃ X₁₄ X₁₅ X₁₆ X₁₇ X₁₈ X₁₉ X₂₀ X₂₁ X₂₂ X₂₃ (SEQ ID NO: 14), wherein X₁ represents tryptophan, X₂ represents any amino acid, optionally an isoleucine, X₃ represents asparagine or optionally glutamine, X₄ represents threonine, X₅ represents any amino acid residue, optionally tyrosine or optionally phenylalanine, X₆ represents any amino acid, optionally threonine, optionally serine, optionally asparagine, alanine or glycine, optionally residues other than large or hydrophobic resides, X₇ represents any amino acid, optionally glycine, optionally alanine, serine, threonine, asparagine or glutamine, optionally a residue other than aspartic acid or glutamic acid, optionally a residue other than lysine or arginine, X₈ represents glutamic acid, optionally aspartic acid, X₉ represents any amino acid, optionally proline, X₁₀ represents any amino acid, optionally threonine, optionally serine, asparagine, glutamine, histidine, glutamic acid, aspartic acid, arginine, lysine, alanine or tyrosine, optionally any residue other than a hydrophobic residue or proline, X₁₁ represents a tyrosine, each of X₁₂, X₁₃, X₁₄, X₁₅ and X₁₆ each represent any amino acid, X₁₇ represents glycine or another residue which does not introduce steric hindrance that reduces antigen binding, X₁₀ represents any amino acid, optionally arginine, X₁₉ represents phenylalanine or another hydrophobic residue capable of maintaining the beta-strand position and V_{H} domain structure integrity, X₂₀ represents alanine or valine, or optionally leucine, optionally a hydrophobic residue, X₂₁ represents phenylalanine or another hydrophobic residue capable of maintaining the beta-strand position and V_{H} domain structure integrity, X₂₂ represents serine, and wherein and X₂₃ represents any amino acid, optionally leucine, optionally alanine, valine or threonine. In one embodiment, the CDR2-FR3 segment further comprises residues at Kabat positions 72, 72a and 72b having the formula X₂₄ X₂₅ X₂₆, wherein X₂₄ represents aspartic acid, glutamic acid or alanine, X₂₅ represents any amino acid, optionally alanine or threonine, or a conservative substitution thereof, and X₂₆ represents serine, optionally alanine, or a conservative substitution thereof.

In one embodiment, the CDR3 comprises a first aromatic amino acid residue that is capable of contacting an amino acid residue in CD39 and a second aromatic amino acid residue that is capable of contacting an amino acid residue in the V_{L}, wherein the first and second aromatic residues are at any of Kabat positions 100, 100b, 100c, 100d, 100e and/or 100f. In one embodiment the Kabat CDR3 (e.g. Kabat positions 100 to 100f, to the extent residues are present at these positions) comprises a sequence of amino residues having the formula X₁ X₂ X₃ X₄ X₅ (SEQ ID NO: 15), wherein any two, three or more of X₁, X₂, X₃, X₄ and X₅ represent an aromatic amino acid.

In one embodiment, provided is an antigen-binding protein capable of binding to and inhibiting the activity of a human CD39 polypeptide, the protein comprising a V_{H} that binds CD39 and a V_{L} (e.g., a V_{L} that binds the CDR3 of the V_{H}), wherein the V_{H} comprises:

(a) a CDR1 capable of contacting the N-terminal domain of CD39, optionally comprising a residue at Kabat position 33, optionally at both positions 31 and 33, that is capable of contacting amino acid residues in CD39;

(b) a CDR2-FR3 domain comprising:
a. a segment comprising amino acid residues capable of contacting the N-terminal domain of CD39, optionally wherein the segment comprises residues within Kabat positions 50-56, optionally wherein the segment comprises one, two, three, four or more of (or all of) the residues at Kabat positions 50, 52, 52a, 53 and/or 56, optionally wherein the residue at position 53 aromatic is an amino acid residue;
b. a segment comprising amino acid residues capable of contacting the C-terminal domain of CD39, optionally wherein the segment comprises residues within Kabat positions 59-71, optionally wherein the segment comprises one, two, three, four or more of (or all of) the residues at Kabat positions 59, 65, 67, 68, 69, 70 and/or 71, optionally further the residue at Kabat position 54, optionally further the residues at Kabat positions 72, 72a and/or 72b; and

(c) a CDR3 (e.g. according to Kabat) capable of contacting the N-terminal domain of CD39, optionally capable of contacting the N-terminal domain of CD39 and the V_{L}, optionally comprising a first aromatic amino acid residue that is capable of contacting an amino acid residue in CD39 and a second aromatic amino acid residue that is capable of contacting an amino acid residue in the V_{L}, wherein the first and second aromatic residues are at any of Kabat positions 100, 100b, 100c, 100d, 100e and/or 100f (to the extent a residue is present at the particular Kabat position).

Optionally, the CDR3 further comprises a further (third) aromatic amino acid residue, and optionally further a fourth aromatic amino acid residue, wherein the third aromatic residue (and fourth aromatic residue, where present) is capable of contacting an amino acid residue in the V_{L}, wherein the third aromatic residue (and fourth residue, where present) is at any of Kabat positions 100, 100b, 100c, 100d, 100e and/or 100f.

In one embodiment, provided is an antigen-binding protein capable of binding to and inhibiting the activity of a human CD39 polypeptide, the protein comprising a V_{H} that binds CD39 and a V_{L} (e.g., a V_{L} that binds the CDR3 of the V_{H}), wherein the V_{H} comprises:
(a) a CDR1 (e.g. according to Kabat) capable of contacting the N-terminal domain of CD39, optionally wherein the residues at Kabat position 31, 32 and 33 have the formula X₁ X₂ X₃, wherein X₁ represents any amino acid, optionally a histidine or asparagine, or optionally a conservative substitution thereof, X₂ represents any amino acid, optionally an aromatic residue, optionally a tyrosine or a conservative substitution thereof, or optionally an amino acid residue other than a proline or glycine, and X₃ represents glycine, or another amino acid that avoids steric hindrance;
(b) a CDR2-FR3 segment (e.g. according to Kabat) capable of contacting the C-terminal domain of CD39, optionally wherein the residues at Kabat position 50-71 having the formula X₁ X₂ X₃ X₄ X₅ X₆ X₇ X₈ X₉ X₁₀ X₁₁ X₁₂ X₁₃ X₁₄ X₁₅ X₁₆ X₁₇ X₁₈ X₁₉ X₂₀ X₂₁ X₂₂ X₂₃ (SEQ ID NO: 14), wherein X₁ represents tryptophan, X₂ represents any amino acid, optionally an isoleucine, X₃ represents asparagine or optionally glutamine, X₄ represents threonine, X₅ represents any amino acid residue, optionally tyrosine or optionally phenylalanine, X₆ represents any amino acid, optionally threonine, optionally serine, optionally asparagine, alanine or glycine, optionally a residue other than a large or hydrophobic residue, X₇ represents any amino acid, optionally glycine, optionally alanine, serine, threonine, asparagine or glutamine, optionally residues other than aspartic acid or glutamic acid, optionally a residue other than lysine or arginine, X₈ represents glutamic acid, optionally aspartic acid, X₉ represents any amino acid, optionally proline, X₁₀ represents any amino acid, optionally threonine, optionally serine, asparagine, glutamine, histidine, glutamic acid, aspartic acid, arginine, lysine, alanine or tyrosine, optionally any residue other than a hydrophobic residue or proline, X₁₁ represents a tyrosine, each of X₁₂, X₁₃, X₁₄, X₁₅ and X₁₆ each represent any amino acid, X₁₇ represents glycine or another residue which does not introduce steric hindrance that reduces antigen binding, X₁₈ represents any amino acid, optionally arginine, X₁₉ represents phenylalanine or another hydrophobic residue capable of maintaining the beta-strand position and V_{H} domain structure integrity, X₂₀ represents alanine or valine, or optionally leucine, optionally a hydrophobic residue, X₂₁ represents phenylalanine or another hydrophobic residue capable of maintaining the beta-strand position and V_{H} domain structure integrity and X₂₂ represents serine, optionally further wherein and X₂₃ represents any amino acid, optionally leucine, optionally alanine, valine or threonine, optionally wherein the CDR2-FR3 segment further comprises residues at Kabat positions 72, 72a and 72b having the formula X₂₄ X₂₅ X₂₆, wherein X₂₄ represents aspartic acid, glutamic acid or alanine, X₂₅ represents any amino acid, optionally alanine or threonine, or a conservative substitution thereof, and X₂₆ represents serine, optionally alanine, or a conservative substitution thereof; and
(c) a CDR3 (e.g. according to Kabat) capable of contacting the N-terminal of CD39, optionally capable of contacting the N-terminal domain of CD39 and the V_{L}, optionally wherein the residues at Kabat position 95-102 have the formula X₁ X₂ X₃ X₄ X₅ X₆ X₇ X₉ X₉ X₁₉ X₉ X₁₀ X₁₁ X₁₂ X₁₃ X₁₄ (SEQ ID NO: 16), wherein
   X₁ represents arginine or lysine, or optionally a conservative substitution thereof,
   X₂ represents any amino acid, optionally arginine, optionally lysine or alanine, or optionally a conservative substitution thereof,
   X₃ represents any amino acid residue, optionally a residue comprising an aromatic ring, optionally a tyrosine,
   X₄ represents any amino acid, optionally glutamic acid or tyrosine, or optionally a conservative substitution thereof, or an amino acid residue other than proline or glycine,
   X₅ represents glycine, optionally arginine, or optionally a conservative substitution thereof,
   X₆ represents any amino acid, optionally asparagine, serine or tyrosine, or optionally a conservative substitution thereof,
   X₇ represents any amino acid, optionally tyrosine, asparagine or aspartic acid, or optionally a conservative substitution thereof, optionally an amino acid residue other than proline or glycine,
   X₈ represents valine or optionally alanine, isoleucine or leucine, optionally an aromatic amino acid, optionally tyrosine,
   X₉ represents any amino acid, optionally an aromatic amino acid, optionally phenylalanine, optionally tyrosine, optionally valine or a conservative substitution thereof,
   X₁₀ represents tyrosine, optionally phenylalanine, optionally methionine, or optionally a conservative substitution thereof,
   X₁₁ is absent or represents any amino acid, optionally tyrosine, optionally phenylalanine, optionally tryptophan, or optionally a conservative substitution thereof, optionally an amino acid residue other than P, G, E or D, or other than a small hydrophobic residue (e.g. T, S),
   X₁₂ is absent or represents any amino acid, optionally phenylalanine, or optionally a conservative substitution thereof,
   X₁₃ represents any amino acid, optionally aspartic acid, or optionally a conservative substitution thereof, optionally a serine, optionally a threonine, optionally a glutamic acid, optionally an asparagine, optionally a residue other than a large and hydrophobic residue, and
   X₁₄ represents any amino acid, optionally tyrosine or optionally a conservative substitution thereof, optionally an aromatic amino acid, optionally a non-aromatic amino acid.

In one embodiment, provided is an antigen-binding protein capable of binding to and inhibiting the activity of a human CD39 polypeptide, the protein comprising a V_{H} that binds CD39 and a V_{L} (e.g., a V_{L} that binds the CDR3 of the V_{H}), wherein the V_{H} comprises:

(a) a CDR1 (e.g. according to Kabat) capable of contacting the N-terminal domain of CD39, optionally wherein the residues at Kabat position 31, 32 and 33 have the formula X₁ X₂ X₃, wherein X₁ represents any amino acid, optionally a histidine or asparagine, or optionally a conservative substitution thereof, X₂ represents any amino acid, optionally an aromatic residue, optionally a tyrosine or a conservative substitution thereof, or optionally an amino acid residue other than a proline or glycine, and X₃ represents glycine, or another amino acid that avoids steric hindrance;

(b) a CDR2-FR3 segment (e.g. according to Kabat) capable of contacting the C-terminal domain of CD39, optionally wherein the residues at Kabat position 59-71 have the formula X₁ X₂ X₃ X₄ X₅ X₆ X₇ X₈ X₉ X₁₀ X₁₁ X₁₂ X₁₃ (SEQ ID NO: 12), wherein X₁ represents a tyrosine, each of X₂, X₃, X₄, X₅ and X₆ each represent any amino acid, X₇ represents glycine or another residue which does not introduce steric hindrance that reduces antigen binding, X₈ represents any amino acid, X₉ represents phenylalanine or another hydrophobic residue capable of maintaining the beta-strand position and V_{H} domain structure integrity, X₁₀ represents alanine or valine, or optionally leucine, optionally threonine, optionally a hydrophobic residue, X₁₁ represents phenylalanine or another hydrophobic residue (e.g. isoleucine) capable of maintaining the beta-strand position and V_{H} domain structure integrity and X₁₂ represents serine, optionally further wherein and X₁₃ represents any amino acid, optionally leucine, optionally alanine, valine, threonine or arginine; and
(c) a CDR3 (e.g. according to Kabat) capable of contacting the N-terminal of CD39, optionally capable of contacting the N-terminal domain of CD39 and the V_{L}, optionally wherein the residues at Kabat position 95-102 have the formula X₁ X₂ X₃ X₄ X₅ X₆ X₇ X₈ X₉ X₁₀ X₉ X₁₀ X₁₁ X₁₂ X₁₃ X₁₄(SEQ ID NO: 16), wherein
X₁ represents arginine, lysine or alanine, or optionally a conservative substitution thereof,
X₂ represents any amino acid, optionally arginine, optionally lysine or alanine, optionally tyrosine, or optionally a conservative substitution thereof,
X₃ represents any amino acid residue, optionally a residue comprising an aromatic ring, optionally a tyrosine,
X₄ represents any amino acid, optionally glutamic acid, tyrosine or asparagine, or optionally a conservative substitution thereof, or an amino acid residue other than proline or glycine,
X₅ represents glycine, optionally arginine, optionally asparagine, or optionally a conservative substitution thereof,
X₆ represents any amino acid, optionally asparagine, serine or tyrosine, or optionally a conservative substitution thereof,
X₇ represents any amino acid, optionally tyrosine, asparagine or aspartic acid, or optionally a conservative substitution thereof, optionally an amino acid residue other than proline or glycine,
X₈ represents valine or optionally alanine, isoleucine, glycine or leucine, optionally an aromatic amino acid, optionally tyrosine,
X₉ represents any amino acid, optionally an aromatic amino acid, optionally phenylalanine, optionally tyrosine, optionally valine, optionally leucine or a conservative substitution thereof,
X₁₀ represents tyrosine, optionally phenylalanine, optionally methionine, or optionally a conservative substitution thereof,
X₁₁ is absent or represents any amino acid, optionally tyrosine, optionally phenylalanine, optionally tryptophan, optionally alanine, or optionally a conservative substitution thereof, optionally an amino acid residue other than P, G, E or D, or other than a small hydrophobic residue (e.g. T, S),
X₁₂ is absent or represents any amino acid, optionally phenylalanine, optionally methionine, or optionally a conservative substitution thereof,
X₁₃ represents any amino acid, optionally aspartic acid, or optionally a conservative substitution thereof, optionally a serine, optionally a threonine, optionally a glutamic acid, optionally an asparagine, optionally a residue other than a large and hydrophobic residue, and
X₁₄ represents any amino acid, optionally tyrosine or optionally a conservative substitution thereof, optionally an aromatic amino acid, optionally a non-aromatic amino acid, optionally alanine.

In one aspect of any embodiment herein, any amino acid residue in a V_{H} or V_{L} can be specified to be a residue that maintains V domain (e.g. V_{H} or V_{L} respectively) domain structure integrity. In one aspect of any embodiment herein, an amino acid residue in a V_{H} or V_{L} can that contacts an antigen or binding partner (e.g. CD39, a V_{L} or a V_{L}) can be specified to be a residue which does not introduce steric hindrance that reduces antigen or binding partner binding.

In one aspect, the binding molecule or antigen-binding fragment thereof comprises human framework regions, e.g. the molecule comprises a V_{H} comprising human V_{H} FR₁, FR₂, FR3 and FR₄ amino acid sequences (optionally comprising one or more amino acid substitutions), and/or a V_{L} comprising human V_{L} FR₁, FR₂, FR₃ and FR4 amino acid sequences (optionally comprising one or more amino acid substitutions).

In one embodiment, the CDR2 comprises an aromatic amino acid residue that contacts an amino acid residues V95, Q96 and/or L137 of CD39. In one embodiment, the aromatic amino acid residue is an aromatic residue, e.g. a tyrosine, optionally a phenylalanine.

In one embodiment, the V_{H} comprises a FR3 comprising an amino acid residue that contacts CD39, optionally wherein the residue in the V_{H} is a leucine at Kabat position 71.

In one embodiment, the V_{H} comprises an amino acid residue in FR1 at Kabat position 19 that contacts CD39, optionally wherein the residue is a lysine.

In one embodiment, the V_{L} comprises a FR2 comprising an aromatic amino acid residue that contacts the CDR3 of the V_{H}, optionally wherein the residue in the V_{L} is at Kabat position 49. Optionally the residue at V_{L} Kabat position is an aromatic residue, optionally further wherein a V_{H} comprises an aromatic residue (e.g. at Kabat 100e) capable of forming a pi stacking interaction therewith.

In one embodiment, the V_{H} CDR3 comprises an amino acid residue(s) comprising an aromatic ring (optionally tyrosine, histidine, tryptophan or phenylalanine), capable of forming a pi interaction (e.g., attractive, noncovalent interactions between an aromatic ring and an binding partner, for example in an amide pi-stacked interaction, a pi-pi stacked interaction or a pi-donor interaction) with an amino acid residue (e.g. an aromatic residue) in the V_{L} polypeptide. In one embodiment, the residue(s) in the CDR3 comprising an aromatic ring is a tyrosine, e.g. at Kabat position 100e. In one embodiment, the residue(s) in the V_{L} comprising an aromatic ring is a tyrosine, e.g. a tyrosine at Kabat position 49 in the V_{L}.

In one embodiment, the V_{H} CDR3 comprises an amino acid residue(s) comprising an aromatic ring (optionally tyrosine, histidine, tryptophan or phenylalanine), capable of forming a pi interaction (e.g., attractive, noncovalent interactions between an aromatic ring and an binding partner, for example in an amide pi-stacked interaction) with an amino acid residue in the CD39 polypeptide (e.g. at residue Q96 in CD39 of SEQ ID NO: 1). In one embodiment, the residue comprising an aromatic ring is a phenylalanine.

In one embodiment, the V_{H} CDR3 comprises (e.g. at Kabat position 100e) an amino acid residue comprising an aromatic ring (e.g. a tyrosine, optionally a phenylalanine), which forms or is capable of forming a pi-pi stacking interaction with an amino acid residue comprising an aromatic ring in the V_{L}, e.g. the tyrosine at Kabat framework (FR2) position 49 in the V_{L}. In one embodiment, the V_{H} CDR3 comprises (e.g. at Kabat position 100f) an amino acid residue comprising an aromatic ring (e.g. a phenylalanine, optionally a tyrosine), which is capable of a pi-donor interaction with an amino acid residue comprising an aromatic ring in the V_{L}, e.g. a glutamine at Kabat CDR3 position 89 in the V_{L}.

In one embodiment, the V_{H} CDR3 comprises (i) a first amino acid residue comprising an aromatic ring, which is capable of forming a pi-stacking interaction with an amino acid residue in the V_{L}, (ii) a second an amino acid residue comprising an aromatic ring, which is capable of forming a pi interaction (e.g. an amide pi-stacked interaction) with an amino acid residue comprising an aromatic ring in the CD39 polypeptide. In one embodiment, the first and/or second residue in the CDR3 comprising an aromatic ring is a tyrosine.

In one embodiment, the V_{H} CDR3 comprises (i) a first and second amino acid residue comprising an aromatic ring (optionally a tyrosine or phenylalanine), which has formed or is capable of a pi (e.g. pi-stacked) interaction with an amino acid residue in the V_{L}; (ii) a third amino acid residue comprising an aromatic ring (optionally a tyrosine), which has formed or is capable of a pi interaction with an amino acid residue in the CD39 polypeptide. In one embodiment, one of the first and second residue comprising an aromatic ring in the V_{H} CDR3 is capable of or has formed a pi-stacking interaction with an amino acid residue comprising an aromatic ring in the V_{L}. In one embodiment, the first residue in the CDR3 comprising an aromatic ring is a tyrosine and the second residue in the CDR3 comprising an aromatic ring is a phenylalanine. Optionally, the third amino acid residue comprising an aromatic ring is a tyrosine.

An exemplary V_{L} can comprise:
a CDR1 comprising a residue, e.g. at one, two, three of four of Kabat positions 31, 32, 33 and/or 34, capable of contacting the CDR3 of the V_{H};
a FR2 comprising an aromatic residue, optionally a tyrosine, at Kabat position 49 capable of contacting the CDR3 of the V_{H}; and/or
a CDR3 comprising a residue, e.g. at Kabat positions 89 and/or 91, capable of contacting the CDR3 of the V_{H}.

In one embodiment, the invention provides a binding molecule or antigen-binding fragment thereof capable of binding to and inhibiting the activity of CD39, comprising a V_{H} of any of the embodiment herein, and a V_{L}, wherein the V_{L} comprises:
a CDR1 comprising a residue at Kabat positions 31, 32, 33 and/or 34 capable of contacting the CDR3 of the V_{H};
a FR2 comprising an aromatic residue, optionally a tyrosine, at Kabat position 49; and/or
a CDR3 comprising a residue at Kabat positions 89 and/or 91 capable of contacting the CDR3 of the V_{H}.

In one embodiment, the invention provides a binding molecule or antigen-binding fragment thereof capable of binding to and inhibiting the activity of CD39, comprising an antibody V_{H} and an antibody V_{L},
wherein the V_{H} comprises the amino acid sequence of Formula I:

[FR₁]CDR1[FR₂]CDR2[FR₃]CDR3[FR₄] (Formula I)
wherein [FR₁], [FR₂], [FR₃] and [FR₄] represent V_{H} framework regions and CDR1, CDR2 and CDR3 represent V_{H} CDRs, wherein:
   CDR1 comprises a residue, optionally at Kabat positions 31 and/or 33, that is capable of contacting the N-terminal domain of CD39,
   CDR2 comprises residues capable of contacting CD39, optionally the N-terminal domain of CD39, optionally wherein two, three, four or five of Kabat positions 50, 52, 52a, 53 and 56 are capable of contacting CD39, optionally in the N-terminal domain, optionally wherein the residue at position 53 comprises an aromatic ring, optionally tyrosine, optionally further wherein a residue in the Kabat CDR2 (e.g. at one, two or three of Kabat positions 57, 59 and/or 65), in combination with residues in the Kabat FR3 (e.g. at one, two or three of Kabat positions 67, 68, 69, 70 and/or 71) are capable of contacting the C-terminal domain of CD39,
   CDR3 comprises an aromatic residue capable of contacting CD39, optionally in the N-terminal domain of CD39, optionally wherein the CDR3 further comprises an aromatic residue capable of contacting the V_{L}, optionally wherein the aromatic residue(s) is/are at any of Kabat positions 100, 100b, 100c, 100d, 100e and/or 100f (to the extent residues are present at the particular position), optionally wherein the aromatic residue capable of contacting the V_{L} is a tyrosine or a phenylalanine and optionally wherein the aromatic residue capable of contacting CD39 is a tyrosine or a phenylalanine; and
wherein the V_{L} comprises the amino acid sequence of Formula II:

[FR₁]CDR1[FR₂]CDR2[FR₃]CDR3[FR₄] (Formula II)
wherein [FR₁], [FR₂], [FR₃] and [FR₄] represent V_{L} framework regions and CDR1, CDR2 and CDR3 represent V_{L} CDRs, wherein:
   CDR1 comprises a residue, optionally at Kabat positions 31, 32, 33 and/or 34, capable of contacting the CDR3 of the V_{H};
   FR2 comprises a residue, optionally an aromatic residue at Kabat position 49, capable of contacting the CDR3 of the V_{H}; and
   CDR3 comprises a residue, optionally at Kabat positions 89 and/or 91, capable of contacting the CDR3 of the V_{H}.

In one embodiment, the V_{H} CDR1 contacts the N-terminal domain of CD39. In one embodiment, the V_{H} CDR2 contacts the C-terminal domain of CD39. In one embodiment, the V_{H} CDR3 contacts the N-terminal domain of CD39.

Optionally, in formula I and/or II, any CDR can be defined as further described herein.

In one embodiment, the V_{H} of formula I comprises a segment of residues within Kabat positions 59-71 (CDR2-FR3), optionally within 59-72b, that are capable of contacting amino acid residues in the C-terminal domain of CD39, optionally further wherein residues within Kabat positions 59-71 contact the glycan at residue N292 of CD39. For example, the Kabat CDR2 and FR3 can comprise residues at Kabat positions 59, 65, 67, 68, 69, 70 and/or 71, and optionally further at residue 72, 72a and/or 72b that are capable of contacting the C-terminal domain of CD39, e.g. including amino acid resides in CD39 and the glycan at N292 of the CD39 polypeptide.

In one embodiment, the V_{L} comprises a CDR1 wherein the residues at Kabat positions 31-34 have the formula X₁ X₂ X₃ X₄, wherein X₁ represents a serine, optionally a threonine, alanine or asparagine, or optionally a conservative substitution thereof, or optionally a residue other than lysine, arginine, isoleucine or leucine, optionally a residue other than phenylalanine, tyrosine or tryptophan, X₂ represents a tyrosine, or optionally alanine or asparagine, or a conservative substitution thereof, X₃ represents phenylalanine or optionally leucine, isoleucine, methionine, valine, tryptophan, optionally a residue other than aspartic acid, glutamic acid, asparagine or lysine, and X₄ represents serine, optionally alanine, optionally a small residue (e.g. alanine, threonine, glycine, asparagine or histidine), optionally other than a large hydrophobic residue.

In one embodiment, the V_{L} comprises a CDR1 wherein the residues at Kabat positions 31-34 have the formula X₁ X₂ X₃ X₄, wherein X₁ represents a threonine or a conservative substitution thereof, X₂ represents alanine or asparagine, or a conservative substitution thereof, X₃ represents valine or a conservative substitution thereof, and X₄ represents alanine or a conservative substitution thereof.

In one embodiment, the V_{L} comprises a CDR1 wherein the residues at Kabat position 24 to 34 have the formula X₁ X₂ X₃ X₄ X₅ X₆ X₇ X₈ X₉ X₁₀ X₉ X₁₀ X₁₁ (SEQ ID NO: 17), wherein
X₁ represents any amino acid, optionally arginine or lysine, or a conservative substitution thereof,
X₂ represents any amino acid, optionally alanine, or a conservative substitution thereof,
X₃ represents any amino acid, optionally serine, or a conservative substitution thereof,
X₄ represents any amino acid, optionally glutamic acid or histidine, or a conservative substitution thereof,
X₅ represents any amino acid, optionally asparagine or aspartic acid, or a conservative substitution thereof,
X₆ represents any amino acid, optionally isoleucine or valine, or a conservative substitution thereof,
X₇ represents any amino acid, optionally tyrosine or glycine, or a conservative substitution thereof,
X₈ represents serine or threonine, or a conservative substitution thereof,
X₉ represents tyrosine, alanine or asparagine, or a conservative substitution thereof,
X₁₀ represents a hydrophobic residue, optionally a phenylalanine, isoleucine or valine, or a conservative substitution thereof, and
X₁₁ represents serine, histidine or alanine, or a conservative substitution thereof.

In one embodiment, the V_{L} comprises a CDR2 wherein the residues at Kabat position 50-56 have the formula X₁ X₂ X₃ X₄ X₅ X₆ X₇ (SEQ ID NO: 18), wherein
X₁ represents any amino acid, optionally threonine, serine or lysine, or a conservative substitution thereof,
X₂ represents any amino acid, optionally alanine, or a conservative substitution thereof,
X₃ represents any amino acid, optionally lysine or serine, or a conservative substitution thereof,
X₄ represents any amino acid, optionally threonine, tyrosine or asparagine, or a conservative substitution thereof,
X₅ represents any amino acid, optionally leucine or arginine, or a conservative substitution thereof,
X₆ represents any amino acid, optionally alanine or tyrosine, or a conservative substitution thereof, and
X₇ represents any amino acid, optionally glutamic acid, threonine or serine, or a conservative substitution thereof.

In one embodiment, the V_{L} comprises a CDR2 wherein the residues at Kabat position 50-56 have the formula X₁ X₂ X₃ X₄ X₅ X₆ X₇ (SEQ ID NO: 19), wherein:
X₁ represents serine, or a conservative substitution thereof,
X₂ represents alanine, or a conservative substitution thereof,
X₃ represents serine, or a conservative substitution thereof,
X₄ represents tyrosine, or a conservative substitution thereof,
X₅ represents arginine, or a conservative substitution thereof,
X₆ represents tyrosine, or a conservative substitution thereof, and
X₇ represents threonine, optionally serine, or a conservative substitution thereof.

In one embodiment, the V_{L} comprises a FR2 comprising a tyrosine, or optionally a phenylalanine, at Kabat position 49.

In one embodiment, the V_{L} comprises a CDR3 wherein the residues at Kabat position 89-91 have the formula X₁ X₂ X₃, wherein X₁ represents any amino acid, optionally a glutamine, optionally a histidine, or a conservative substitution thereof, X₂ represents any amino acid, optionally a glutamine or histidine, or a conservative substitution thereof, and X₃ represents histidine, or optionally tyrosine, or a conservative substitution thereof, or optionally asparagine, or optionally a residue other than a large or hydrophobic residue.

Optionally, in one embodiment, the V_{L} comprises residues at any one, two, three or four of Kabat positions 94, 95, 96 and 97 (Kabat CDR3) that contact the V_{H} domain framework.

In one embodiment, the V_{L} comprises a CDR3 wherein the residues at Kabat position 89 is a glutamine or histidine, or a conservative substitution thereof, the residue at position 91 is a tyrosine or histidine, or a conservative substitution thereof, the residue at position 95 is a proline, or a conservative substitution thereof, and the residue at position 96 is a tyrosine, or a conservative substitution thereof.

In one embodiment, the V_{L} comprises a CDR3 wherein the residues at Kabat position 89-97 have the formula X₁ X₂ X₃ X₄ X₅ X₆ X₇ X₉ X₉X₁₉ (SEQ ID NO: 20), wherein
X₁ represents glutamine or histidine, or a conservative substitution thereof,
X₂ represents any amino acid, optionally glutamine or histidine, or a conservative substitution thereof,
X₃ represents tyrosine, histidine or threonine or a conservative substitution thereof,
X₄ represents any amino acid, optionally tyrosine, asparagine or tryptophan, or a conservative substitution thereof,
X₅ represents any amino acid, optionally valine or asparagine, or a conservative substitution thereof,
X₆ represents any amino acid, optionally threonine, tyrosine or aspartic acid, or a conservative substitution thereof,
X₇ represents any amino acid, optionally proline, or a conservative substitution thereof,
X₈ is absent or represents any one or more amino acids, optionally a proline, or a conservative substitution thereof,
X₉ represents any amino acid, optionally an aromatic residue, optionally tyrosine, phenylalanine, or a conservative substitution thereof, and
X₁₀ represents any amino acid, optionally threonine, or a conservative substitution thereof.

In one embodiment, the V_{L} comprises:
a CDR1 wherein the residues at Kabat position 31, 32, 33 and 34 have the formula X₁ X₂ X₃ X₄, wherein X₁ represents a threonine or a conservative substitution thereof, X₂ represents alanine or asparagine, or a conservative substitution thereof, X₃ represents valine or a conservative substitution thereof, and X₄ represents alanine or a conservative substitution thereof;
a FR2 comprising an aromatic residue, optionally a tyrosine, at Kabat position 49; and
a CDR3 wherein the residues at Kabat position 89 is a glutamine or histidine, or a conservative substitution thereof, the residue at position 91 is a tyrosine or histidine (optionally the residue at position 91 is an aromatic residue), or a conservative substitution thereof, optionally wherein the residue at position 95 is a proline, or a conservative substitution thereof, optionally wherein the residue at position 96 is a tyrosine, or a conservative substitution thereof.

In another embodiment, the V_{L} comprises:
a CDR1 wherein the residues at Kabat position 31, 32, 33 and 34 have the formula X₁ X₂ X₃ X₄, wherein X₁ represents a serine or a conservative substitution thereof, X₂ represents tyrosine, or a conservative substitution thereof, X₃ represents a hydrophobic residue, optionally a phenylalanine or an isoleucine, and X₄ represents any amino acid, optionally a serine or histidine or a conservative substitution thereof;
a FR2 comprising an aromatic residue, optionally a tyrosine, at Kabat position 49; and
a CDR3 wherein the residues at Kabat position 89 is a glutamine or histidine, or a conservative substitution thereof, optionally the residue at position 91 is a tyrosine, histidine, threonine, or a conservative substitution thereof, optionally wherein the residue at position 95 is a proline, or a conservative substitution thereof, optionally wherein the residue at position 96 is an aromatic residue, optionally tyrosine or phenylalanine.

It will be appreciated that the crystal structures disclosed can be used to guide design of FR and CDR amino acid sequences while retaining the desired functional properties.

In certain embodiment, the binding molecules and domains can be derived from immunoglobulin variable domains, for example in the form of associated V_{L} and V_{H} domains found on two polypeptide chains, or a single chain antigen binding domain such as a scFv, a V_{H} domain, a V_{L} domain, a dAb, a V-NAR domain or a V_{H}H domain.

In one aspect, the CD39 binding agent or molecule is an antibody selected from a fully human antibody, a humanized antibody, and a chimeric antibody.

In one aspect, the agent is a fragment of an antibody comprising a constant or Fc domain derived from a human IgG1 constant or Fc domain, e.g., modified, as further disclosed herein.

In one aspect, the agent comprises an antibody fragment selected from a Fab fragment, a Fab′ fragment, a Fab′-SH fragment, a F(ab)2 fragment, a F(ab′)2 fragment, an Fv fragment, a Heavy chain Ig (a llama or camel Ig), a V_{HH} fragment, a single domain FV, and a single-chain antibody fragment. In one aspect, the agent comprises a synthetic or semisynthetic antibody-derived molecule selected from a scFV, a dsFV, a minibody, a diabody, a triabody, a kappa body, an IgNAR; and a multispecific (e.g. bispecific) antibody. The agent can optionally further comprise an Fc domain.

In one aspect, the antibody is in at least partially purified form.

In one aspect, the antibody is in essentially isolated form.

Antibodies may be produced by a variety of techniques known in the art. Typically, they are produced by selection from an antibody library (e.g. as generated from phage display library), or by immunization of a non-human animal, preferably a mouse, with an immunogen comprising a CD39 polypeptide, preferably a human CD39 polypeptide. The CD39 polypeptide may comprise the full length sequence of a human CD39 polypeptide, or a fragment or derivative thereof, typically an immunogenic fragment, i.e., a portion of the polypeptide comprising an epitope exposed on the surface of cells expressing a CD39 polypeptide. Such fragments typically contain at least about 7 consecutive amino acids of the mature polypeptide sequence, even more preferably at least about 10 consecutive amino acids thereof. Fragments typically are essentially derived from the extra-cellular domain of the receptor. In one embodiment, the immunogen comprises a wild-type human CD39 polypeptide in a lipid membrane, typically at the surface of a cell. In a specific embodiment, the immunogen comprises intact cells, particularly intact human cells, optionally treated or lysed. In another embodiment, the polypeptide is a recombinant CD39 polypeptide.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988), the entire disclosure of which is herein incorporated by reference). The immunogen is suspended or dissolved in a buffer, optionally with an adjuvant, such as complete or incomplete Freund's adjuvant. Methods for determining the amount of immunogen, types of buffers and amounts of adjuvant are well known to those of skill in the art and are not limiting in any way. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with an adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be used as long as they result in the production of B cells expressing an antibody directed to the antigen used in immunization.

In an alternate embodiment, lymphocytes from a non-immunized non-human mammal are isolated, grown in vitro, and then exposed to the immunogen in cell culture. The lymphocytes are then harvested and the fusion step described below is carried out.

For monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the lymphocytes can be fused to an immortal cell line. This is typically a mouse myeloma cell line, although many other immortal cell lines useful for creating hybridomas are known in the art. Murine myeloma lines include, but are not limited to, those derived from MOPC-21 and MPC-11 mouse tumors available from the Salk Institute Cell Distribution Center, San Diego, U.S.A, X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in Goding, “Monoclonal Antibodies: Principles and Practice,” pp. 59-103 (Academic Press, 1986), the disclosure of which is herein incorporated by reference.

The cells are allowed to grow in the selection media for sufficient time for colony formation and antibody production. This is usually between about 7 and about 14 days.

The hybridoma colonies are then assayed for the production of antibodies that specifically bind to CD39 polypeptide gene products. The assay is typically a colorimetric ELISA-type assay, although any assay may be employed that can be adapted to the wells that the hybridomas are grown in. Other assays include radioimmunoassays or fluorescence activated cell sorting. The wells positive for the desired antibody production are examined to determine if one or more distinct colonies are present. If more than one colony is present, the cells may be re-cloned and grown to ensure that only a single cell has given rise to the colony producing the desired antibody. Typically, the antibodies will also be tested for the ability to bind to CD39 polypeptides, e.g., on CD39-expressing cells.

Hybridomas that are confirmed to produce a monoclonal antibody can be grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

Positive wells with a single apparent colony are typically re-cloned and re-assayed to insure only one monoclonal antibody is being detected and produced.

Antibodies may also be produced by selection of combinatorial libraries of immunoglobulins, as disclosed for instance in (Ward et al. Nature, 341 (1989) p. 544, the entire disclosure of which is herein incorporated by reference).

The identification of one or more antibodies that bind(s) to CD39, particularly substantially or essentially the same region on CD39 as monoclonal antibody I-391 or I-392, can be readily determined using any one of a variety of immunological screening assays in which antibody competition can be assessed. Many such assays are routinely practiced and are well known in the art (see, e. g., U.S. Pat. No. 5,660,827, issued Aug. 26, 1997, which is specifically incorporated herein by reference).

For example, where the test antibodies to be examined are obtained from different source animals, or are even of a different Ig isotype, a simple competition assay may be employed in which the control (I-391, for example) and test antibodies are admixed (or pre-adsorbed) and applied to a sample containing CD39 polypeptides. Protocols based upon western blotting and the use of BIACORE analysis are suitable for use in such competition studies.

In certain embodiments, one pre-mixes the control antibodies (I-391 or I-392, for example) with varying amounts of the test antibodies (e.g., about 1:10 or about 1:100) for a period of time prior to applying to the CD39 antigen sample. In other embodiments, the control and varying amounts of test antibodies can simply be admixed during exposure to the CD39 antigen sample. As long as one can distinguish bound from free antibodies (e. g., by using separation or washing techniques to eliminate unbound antibodies) and I-391 from the test antibodies (e. g., by using species-specific or isotype-specific secondary antibodies or by specifically labelling I-391 or I-392 with a detectable label) one can determine if the test antibodies reduce the binding of respective I-391 or I-392 to the antigens. The binding of the (labelled) control antibodies in the absence of a completely irrelevant antibody can serve as the control high value. The control low value can be obtained by incubating the labelled (I-391 or I-392) antibodies with unlabelled antibodies of exactly the same type (I-391 or I-392), where competition would occur and reduce binding of the labelled antibodies. In a test assay, a significant reduction in labelled antibody reactivity in the presence of a test antibody is indicative of a test antibody that recognizes substantially the same epitope, i.e., one that “cross-reacts” or competes with the labelled (I-391 or I-392) antibody. A test antibody can be selected that reduces the binding of I-391 or I-392 to CD39 antigens by at least about 50%, such as at least about 60%, or more preferably at least about 80% or 90% (e. g., about 65-100%), at any ratio of I-391:test antibody between about 1:10 and about 1:100. Preferably, such test antibody will reduce the binding of the respective I-391 or I-392 to the CD39 antigen by at least about 90% (e.g., about 95%).

Competition can also be assessed by, for example, a flow cytometry test. In such a test, cells bearing a given CD39 polypeptide can be incubated first with I-391 or I-392, for example, and then with the test antibody labelled with a fluorochrome or biotin. The antibody is said to compete with I-391 or I-392 if the binding obtained upon preincubation with a saturating amount of the respective I-391 or I-392 is about 80%, preferably about 50%, about 40% or less (e.g., about 30%, 20% or 10%) of the binding (as measured by mean of fluorescence) obtained by the antibody without preincubation with the respective I-391 or I-392. Alternatively, an antibody is said to compete with I-391 or I-392if the binding obtained with a labelled I-391 or I-392 antibody (by a fluorochrome or biotin) on cells preincubated with a saturating amount of test antibody is about 80%, preferably about 50%, about 40%, or less (e.g., about 30%, 20% or 10%) of the binding obtained without preincubation with the test antibody.

A simple competition assay in which a test antibody is pre-adsorbed and applied at saturating concentration to a surface onto which a CD39 antigen is immobilized may also be employed. The surface in the simple competition assay is preferably a BIACORE chip (or other media suitable for surface plasmon resonance analysis). The control antibody (e.g., I-391) is then brought into contact with the surface at a CD39-saturating concentration and the CD39 and surface binding of the control antibody is measured. This binding of the control antibody is compared with the binding of the control antibody to the CD39-containing surface in the absence of test antibody. In a test assay, a significant reduction in binding of the CD39-containing surface by the control antibody in the presence of a test antibody indicates that the test antibody recognizes substantially the same region of CD39 as the control antibody such that the test antibody “cross-reacts” with the control antibody. Any test antibody that reduces the binding of control (such as I-391) antibody to a CD39 antigen by at least about 30% or more, preferably about 40%, can be considered to be an antibody that competes with a control (e.g., I-391). Preferably, such a test antibody will reduce the binding of the control antibody (e.g., I-391) to the CD39 antigen by at least about 50% (e. g., at least about 60%, at least about 70%, or more). It will be appreciated that the order of control and test antibodies can be reversed: that is, the control antibody can be first bound to the surface and the test antibody is brought into contact with the surface thereafter in a competition assay. Preferably, the antibody having higher affinity for the CD39 antigen is bound to the surface first, as it will be expected that the decrease in binding seen for the second antibody (assuming the antibodies are cross-reacting) will be of greater magnitude. Further examples of such assays are provided in, e.g., Saunal (1995) J. Immunol. Methods 183: 33-41, the disclosure of which is incorporated herein by reference.

The antibodies will bind to CD39-expressing cells from an individual or individuals with a disease characterized by expression of CD39-positive cells, i.e. an individual that is a candidate for treatment with one of the herein-described methods using an anti-CD39 antibody. Accordingly, once an antibody that specifically recognizes CD39 on cells is obtained, it can optionally be tested for its ability to bind to CD39-positive cells (e.g. cancer cells). In particular, prior to treating a patient with one of the present antibodies, one may optionally test the ability of the antibody to bind malignant cells taken from the patient, e.g. in a blood sample or tumor biopsy, to maximize the likelihood that the therapy will be beneficial in the patient.

In one embodiment, the antibodies are validated in an immunoassay to test their ability to bind to CD39-expressing cells, e.g. malignant cells. For example, a blood sample or tumor biopsy is performed and tumor cells are collected. The ability of a given antibody to bind to the cells is then assessed using standard methods well known to those in the art. Antibodies may bind for example to a substantial proportion (e.g., 20%, 30%, 40%, 50%, 60%, 70%, 80% or more) of cells known to express CD39, e.g. tumor cells, from a significant percentage of individuals or patients (e.g., 10%, 20%, 30%, 40%, 50% or more). Antibodies can be used for diagnostic purposes to determine the presence or level of malignant cells in a patient, for example as a biomarker to assess whether a patient is suitable for treatment with an anti-CD39 agent, or for use in the herein-described therapeutic methods. To assess the binding of the antibodies to the cells, the antibodies can either be directly or indirectly labelled. When indirectly labelled, a secondary, labelled antibody is typically added.

Determination of whether an antibody binds within an epitope region can be carried out in ways known to the person skilled in the art. As one example of such mapping/characterization methods, an epitope region for an anti-CD39 antibody may be determined by epitope “foot-printing” using chemical modification of the exposed amines/carboxyls in the CD39 protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e. g., Ehring H, Analytical Biochemistry, Vol. 267 (2) pp. 252-259 (1999) Engen, J. R. and Smith, D. L. (2001) Anal. Chem. 73, 256A-265A. Another example of a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e. g., Ernst Schering Res Found Workshop. 2004; (44): 149-67; Huang et al., Journal of Molecular Biology, Vol. 281 (1) pp. 61-67 (1998); and Saito and Patterson, Methods. 1996 June; 9 (3): 516-24.

Epitope mapping/characterization also can be performed using mass spectrometry methods. See, e.g., Downard, J Mass Spectrom. 2000 April; 35 (4): 493-503 and Kiselar and Downard, Anal Chem. 1999 May 1; 71 (9): 1792-1801. Protease digestion techniques also can be useful in the context of epitope mapping and identification. Antigenic determinant-relevant regions/sequences can be determined by protease digestion, e.g. by using trypsin in a ratio of about 1:50 to CD39 or o/n digestion at and pH 7-8, followed by mass spectrometry (MS) analysis for peptide identification. The peptides protected from trypsin cleavage by the anti-CD39 binder can subsequently be identified by comparison of samples subjected to trypsin digestion and samples incubated with antibody and then subjected to digestion by e.g. trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the CD39 polypeptide that is not surface exposed and, accordingly, most likely not relevant in terms of immunogenicity/antigenicity.

Site-directed mutagenesis is another technique useful for elucidation of a binding epitope. For example, in “alanine-scanning”, each residue within a protein segment is replaced with an alanine residue, and the consequences for binding affinity measured. If the mutation leads to a significant reduction in binding affinity, it is most likely involved in binding. Monoclonal antibodies specific for structural epitopes (i.e., antibodies which do not bind the unfolded protein) can be used to verify that the alanine-replacement does not influence overall fold of the protein. See, e.g., Clackson and Wells, Science 1995; 267:383-386; and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron microscopy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, BIACORE) and reflectometric interference spectroscopy (RifS). See, e.g., Fägerstam et al., Journal Of Molecular Recognition 1990; 3:208-14; Nice et al., J. Chroma-togr. 1993; 646:159-168; Leipert et al., Angew. Chem. Int. Ed. 1998; 37:3308-3311; Kröger et al., Biosensors and Bioelectronics 2002; 17:937-944.

It should also be noted that an antibody that binds the same or substantially the same epitope as an antibody can be identified in one or more of the exemplary competition assays described herein.

Upon immunization and production of antibodies in a vertebrate or cell, particular selection steps may be performed to isolate antibodies as claimed. In this regard, in a specific embodiment, the disclosure also relates to methods of producing such antibodies, comprising: (a) immunizing a non-human mammal with an immunogen comprising a CD39 polypeptide; and (b) preparing antibodies from said immunized animal; and (c) selecting antibodies from step (b) that are capable of binding CD39.

Typically, an anti-CD39 antibody provided herein has an affinity for a CD39 polypeptide (e.g., a monomeric CD39 polypeptide as produced in the Examples herein) in the range of about 10⁴ to about 10¹¹ M⁻¹ (e.g., about 10⁸ to about 10¹⁰ M⁻¹). For example, in a particular aspect the disclosure provides Anti-CD39 antibody that have an average disassociation constant (K_{D}) of less than 1×10⁻⁹ M with respect to CD39, as determined by, e.g., surface plasmon resonance (SPR) screening (such as by analysis with a BIAcore™ SPR analytical device). In a more particular exemplary aspect, the disclosure provides anti-CD39 antibodies that have a KD of about 1×10⁻⁸ M to about 1×10⁻¹⁰ M, or about 1×10⁻⁹ M to about 1×10⁻¹¹ M, for CD39.

Antibodies can be characterized for example by a mean KD of no more than about (i.e. better affinity than) 100, 60, 10, 5, or 1 nanomolar, preferably sub-nanomolar or optionally no more than about 500, 200, 100 or 10 picomolar. KD can be determined for example for example by immobilizing recombinantly produced human CD39 proteins on a chip surface, followed by application of the antibody to be tested in solution. In one embodiment, the method further comprises a step (d), selecting antibodies from (b) that are capable of competing for binding to CD39 with antibody I-391.

In one aspect of any of the embodiments, the antibodies prepared according to the present methods are monoclonal antibodies. In another aspect, the non-human animal used to produce antibodies according to the methods herein is a mammal, such as a rodent, bovine, porcine, fowl, horse, rabbit, goat, or sheep.

DNA encoding an antibody that binds an epitope present on CD39 polypeptides is isolated from a hybridoma and placed in an appropriate expression vector for transfection into an appropriate host. The host is then used for the recombinant production of the antibody, or variants thereof, such as a humanized version of that monoclonal antibody, active fragments of the antibody, chimeric antibodies comprising the antigen recognition portion of the antibody, or versions comprising a detectable moiety.

DNA encoding the monoclonal antibodies of the disclosure, e.g., antibody I-391, can be readily isolated and sequenced using conventional procedures (e. g., by using oligonucleotide probes that are capable of binding specifically to genes encoding the heavy and light chains of murine antibodies). In one aspect, provided is a nucleic acid encoding a heavy chain or a light chain of an anti-CD39 antibody of any embodiment herein. Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody. In one embodiment, provided is an isolated nucleic acid sequence encoding a light chain and/or a heavy chain of an antibody (e.g. I-391), as well as a recombinant host cell comprising (e.g. in its genome) such nucleic acid. Recombinant expression in bacteria of DNA encoding the antibody is well known in the art (see, for example, Skerra et al., Curr. Opinion in Immunol., 5, pp. 256 (1993); and Pluckthun, Immunol. 130, p. 151 (1992).

Once antibodies are identified that are capable of binding CD39 and/or having other desired properties, they will also typically be assessed, using methods such as those described herein, for their ability to bind to other polypeptides, including unrelated polypeptides. Ideally, the antibodies bind with substantial affinity only to CD39, and do not bind at a significant level to unrelated polypeptides, or other polypeptides of the NTPDase family, notably CD39-L1, L2, L3 and L4 or NTPDase8. However, it will be appreciated that, as long as the affinity for CD39 is substantially greater (e.g., 10x, 100x, 500x, 1000x, 10,000x, or more) than it is for other, unrelated polypeptides), then the antibodies are suitable for use in the present methods.

In one embodiment, the anti-CD39 antibodies can be prepared such that they do not have substantial specific binding to human Fcγ receptors, e.g., any one or more of CD16A, CD16B, CD32A, CD32B and/or CD64). Such antibodies may comprise constant regions of various heavy chains that are known to lack or have low binding to Fcγ receptors. Alternatively, antibody fragments that do not comprise (or comprise portions of) constant regions, such as F(ab′)2 fragments, can be used to avoid Fc receptor binding. Fc receptor binding can be assessed according to methods known in the art, including for example testing binding of an antibody to Fc receptor protein in a BIACORE assay. Also, generally any antibody IgG isotype can be used in which the Fc portion is modified (e.g., by introducing 1, 2, 3, 4, 5 or more amino acid substitutions) to minimize or eliminate binding to Fc receptors (see, e.g., WO 03/101485, the disclosure of which is herein incorporated by reference). Assays such as cell based assays, to assess Fc receptor binding are well known in the art, and are described in, e.g., WO 03/101485.

In one embodiment, the antibody can comprise one or more specific mutations in the Fc region that result in “Fc silent” antibodies that have minimal interaction with effector cells. Silenced effector functions can be obtained by mutation in the Fc region of the antibodies and have been described in the art: N297A mutation, the LALA mutations, (Strohl, W., 2009, Curr. Opin. Biotechnol. vol. 20(6):685-691); and D265A (Baudino et al., 2008, J. Immunol. 181: 6664-69) see also Heusser et al., WO2012/065950, the disclosures of which are incorporated herein by reference. In one embodiment, an antibody comprises one, two, three or more amino acid substitutions in the hinge region. In one embodiment, the antibody is an IgG1 or IgG2 and comprises one, two or three substitutions at residues 233-236, optionally 233-238 (EU numbering). In one embodiment, the antibody is an IgG4 and comprises one, two or three substitutions at residues 327, 330 and/or 331 (EU numbering). Examples of silent Fc IgG1 antibodies are the LALA mutant comprising L234A and L235A mutation in the IgG1 Fc amino acid sequence. Another example of an Fc silent mutation is a mutation at residue D265, or at D265 and P329 for example as used in an IgG1 antibody as the DAPA (D265A, P329A) mutation (U.S. Pat. No. 6,737,056). Another silent IgG1 antibody comprises a mutation at residue N297 (e.g. N297A, N297S mutation), which results in a glycosylated/non-glycosylated antibodies. Other silent mutations include: substitutions at residues L234 and G237 (L234A/G237A); substitutions at residues S228, L235 and R409 (S228P/L235E/R409K,T,M,L); substitutions at residues H268, V309, A330 and A331 (H268Q/V309L/A330S/A331S); substitutions at residues C220, C226, C229 and P238 (C220S/C226S/C229S/P238S); substitutions at residues C226, C229, E233, L234 and L235 (C226S/C229S/E233P/L234V/L235A; substitutions at residues K322, L235 and L235 (K322A/L234A/L235A); substitutions at residues L234, L235 and P331 (L234F/L235E/P331S); substitutions at residues 234, 235 and 297; substitutions at residues E318, K320 and K322 (L235E/E318A/K320A/K322A); substitutions at residues (V234A, G237A, P238S); substitutions at residues 243 and 264; substitutions at residues 297 and 299; substitutions such that residues 233, 234, 235, 237, and 238 defined by the EU numbering system, comprise a sequence selected from PAAAP, PAAAS and SAAAS (see WO2011/066501).

In one embodiment, the antibody can comprise one or more specific mutations in the Fc region that result in improved stability of an antibody of the disclosure, e.g. comprising multiple aromatic amino acid residues and/or having high hydrophobicity. For example, such an antibody can comprise an Fc domain of human IgG1 origin, comprises a mutation at Kabat residue(s) 234, 235, 237, 330 and/or 331. One example of such an Fc domain comprises substitutions at Kabat residues L234, L235 and P331 (e.g., L234A/L235E/P331S or (L234F/L235E/P331S). Another example of such an Fc domain comprises substitutions at Kabat residues L234, L235, G237 and P331 (e.g., L234A/L235E/G237A/P331S). Another example of such an Fc domain comprises substitutions at Kabat residues L234, L235, G237, A330 and P331 (e.g., L234A/L235E/G237A/A330S/P331S). In one embodiment, the antibody comprises an Fc domain, optionally of human IgG1 isotype, comprising: a L234X, substitution, a L235X₂ substitution, and a P331 X₃ substitution, wherein X₁ is any amino acid residue other than leucine, X₂ is any amino acid residue other than leucine, and X₃ is any amino acid residue other than proline; optionally wherein X₁ is an alanine or phenylalanine or a conservative substitution thereof; optionally wherein X₂ is glutamic acid or a conservative substitution thereof; optionally wherein X₃ is a serine or a conservative substitution thereof. In another embodiment, the antibody comprises an Fc domain, optionally of human IgG1 isotype, comprising: a L234X, substitution, a L235X₂ substitution, a G237X₄ substitution and a P331X₄ substitution, wherein X₁ is any amino acid residue other than leucine, X₂ is any amino acid residue other than leucine, X₃ is any amino acid residue other than glycine, and X₄ is any amino acid residue other than proline; optionally wherein X₁ is an alanine or phenylalanine or a conservative substitution thereof; optionally wherein X₂ is glutamic acid or a conservative substitution thereof; optionally, X₃ is alanine or a conservative substitution thereof; optionally X₄ is a serine or a conservative substitution thereof. In another embodiment, the antibody comprises an Fc domain, optionally of human IgG1 isotype, comprising: a L234X, substitution, a L235X₂ substitution, a G237X₄ substitution, G330X₄ substitution, and a P331X₅ substitution, wherein X₁ is any amino acid residue other than leucine, X₂ is any amino acid residue other than leucine, X₃ is any amino acid residue other than glycine, X₄ is any amino acid residue other than alanine, and X₅ is any amino acid residue other than proline; optionally wherein X₁ is an alanine or phenylalanine or a conservative substitution thereof; optionally wherein X₂ is glutamic acid or a conservative substitution thereof; optionally, X₃ is alanine or a conservative substitution thereof; optionally, X₄ is serine or a conservative substitution thereof; optionally X₅ is a serine or a conservative substitution thereof. In the shorthand notation used here, the format is: Wild type residue: Position in polypeptide: Mutant residue, wherein residue positions are indicated according to EU numbering according to Kabat.

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 21)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In one embodiment, an antibody comprises a heavy chain constant region comprising the amino acid sequence below, or an amino acid sequence at least 90%, 95% or 99% identical thereto but retaining the amino acid residues at Kabat positions 234, 235 and 331 (underlined):

(SEQ ID NO: 22)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E F E G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In one embodiment, an antibody comprises a heavy chain constant region comprising the amino acid sequence below, or an amino acid sequence at least 90%, 95% or 99% identical thereto but retaining the amino acid residues at Kabat positions 234, 235, 237, 330 and 331 (underlined):

(SEQ ID NO: 23)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] E G A P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P S S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

In one embodiment, an antibody comprises a heavy chain constant region comprising the amino acid sequence below, or a sequence at least 90%, 95% or 99% identical thereto but retaining the amino acid residues at Kabat positions 234, 235, 237 and 331 (underlined):

(SEQ ID NO: 24)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G A P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

Fc silent antibodies result in no or low ADCC activity, meaning that an Fc silent antibody exhibits an ADCC activity that is below 50% specific cell lysis. Preferably an antibody substantially lacks ADCC activity, e.g., the Fc silent antibody exhibits an ADCC activity (specific cell lysis) that is below 5% or below 1%. Fc silent antibodies can also result in lack of FcγR-mediated cross-linking of CD39 at the surface of a CD39-expression.

In one embodiment, the antibody has a substitution in a heavy chain constant region at any one, two, three, four, five or more of residues selected from the group consisting of: 220, 226, 229, 233, 234, 235, 236, 237, 238, 243, 264, 268, 297, 298, 299, 309, 310, 318, 320, 322, 327, 330, 331 and 409 (numbering of residues in the heavy chain constant region is according to EU numbering according to Kabat). In one embodiment, the antibody comprises a substitution at residues 234, 235 and 322. In one embodiment, the antibody has a substitution at residues 234, 235 and 331. In one embodiment, the antibody has a substitution at residues 234, 235, 237 and 331. In one embodiment, the antibody has a substitution at residues 234, 235, 237, 330 and 331. In one embodiment, the Fc domain is of human IgG1 subtype. Amino acid residues are indicated according to EU numbering according to Kabat.

In one embodiment, the antibody comprises an Fc domain comprising an amino acid substitution that increases binding to human FcRn polypeptides in order to increase the in vivo half-life of the antibody. Exemplary mutations are described in Strohl, W., 2009, Curr. Opin. Biotechnol. vol. 20(6):685-691, the disclosure of which is incorporated herein by reference. Examples of substitutions used in antibodies of human IgG1 isotype are substitutions at residues M252, S254 and T256; substitutions at residues T250 and M428; substitutions at residue N434; substitutions at residues H433 and N434; substitutions at residues 1307, E380 and N434; substitutions at residues 1307, E380, and N434; substitutions at residues M252, S254, T256, H433, N434 and 436; substitutions at residue 1253; substitutions at residues P257, N434, D376 and N434.

In one embodiment, the antibody comprises an Fc domain comprising an amino acid substitution that confers decreased sensitivity to cleavage by proteases. Matrix metalloproteinases (MMPs) represent the most prominent family of proteinases associated with tumorigenesis. While cancer cells can express MMPs, the bulk of the extracellular MMP is provided by different types of stromal cells that infiltrate the tumor and each produce a specific set of proteinases and proteinase inhibitors, which are released into the extracellular space and specifically alter the milieu around the tumor. The MMPs present in the tumor microenvironment can cleave antibodies within the hinge region and may thus lead to the inactivation of therapeutic antibodies that are designed to function within the tumor site. In one embodiment, the Fc domain comprising an amino acid substitution has decreased sensitivity to cleavage by any one, two, three or more (or all of) of the proteases selected from the group consisting of: GluV8, IdeS, gelatinase A (MMP2), gelatinase B (MMP-9), matrix metalloproteinase-7 (MMP-7), stromelysin (MMP-3), and macrophage elastase (MMP-12). In one embodiment, the antibody decreased sensitivity to cleavage comprises an Fc domain comprising an amino acid substitution at residues E233-L234 and/or L235. In one embodiment, the antibody comprises an Fc domain comprising an amino acid substitution at residues E233, L234, L235 and G236. In one embodiment, the antibody comprises an Fc domain comprising an amino acid substitution at one or more residues 233-238, e.g., such that E233-L234-L235-G236 sequence is replaced by P233-V234-A235 (G236 is deleted). See, e.g., WO99/58572 and WO2012087746, the disclosures of which are incorporated herein by reference.

An antigen-binding compound can at any desired stage be assessed for its ability to inhibit the enzymatic activity of CD39, notably to block the ATPase activity of CD39 and to reduce the production of ADP and AMP (and, together with CD73, adenosine) by a CD39-expressing cell, and in turn restore the activity of and/or relieve the adenosine-mediated inhibition of lymphocytes.

The inhibitory activity (e.g., immune enhancing potential) of an antibody can be assessed for example, in an assay to detect the disappearance (hydrolysis) of ATP and/or the generation of AMP. In one aspect, an assay is used that is insensitive to CD39 down-modulation is used. An example of such an assay is assessing generation of AMP by detection AMP after incubating CD39-expressing cells (e.g. Ramos cells) with a test antibody, and measuring in supernatants the generation of AMP by mass spectrometry (e.g. MALDI TOF). See, e.g., Example 6.

A decrease in hydrolysis of ATP into AMP, and/or a decrease in generation of AMP, in the presence of antibody indicate the antibody inhibits CD39. In one embodiment, an antibody preparation is capable of causing at least a 60% decrease in the enzymatic activity of a CD39 polypeptide, preferably at least a 70%, 80% or 90% decrease in the enzymatic activity of a CD39 polypeptide, as assessed by detecting generation of AMP by detection AMP after incubating CD39-expressing cells (e.g. Ramos cells) with a test antibody, and measuring in supernatants the generation of AMP by mass spectrometry (e.g. MALDI TOF), e.g., as in Example 6.

The activity of an antibody can also be measured in an indirect assay for its ability to modulate the activity of immune cells (e.g. adenosine receptor-expressing immune cells; A2A-receptor expressing cells), for example to relieve the adenosine-mediated inhibition of lymphocyte activity, or to cause the activation of lymphocyte activity. This can be addressed, for example, using a cytokine-release assay. In another example, an antibody can be evaluated in an indirect assay for its ability to modulate the proliferation of lymphocytes.

The antibody can be tested for its ability to internalize or to induce down-modulation of CD39, e.g. whether by internalization or induction of CD39 shedding from the cell surface. Whether an anti-CD39 antibody internalizes upon binding CD39 on a mammalian cell, or whether a CD39 polypeptide undergoes intracellular internalization (e.g. upon being bound by an antibody) can be determined by various assays including those described in the experimental examples herein (e.g., Example 5). In other examples, to test internalization in vivo, the test antibody is labeled and introduced into an animal known to have CD39 expressed on the surface of certain cells. The antibody can be radiolabeled or labeled with fluorescent or gold particles, for instance. Animals suitable for this assay include a mammal such as a nude mouse that contains a human CD39-expressing B cells, T cells, TReg cells, tumor transplant or xenograft, or a mouse into which cells transfected with human CD39 have been introduced, or a transgenic mouse expressing the human CD39 transgene. Appropriate controls include animals that did not receive the test antibody or that received an unrelated antibody, and animals that received an antibody to another antigen on the cells of interest, which antibody is known to be internalized upon binding to the antigen. The antibody can be administered to the animal, e.g., by intravenous injection. At suitable time intervals, tissue sections of the animal can be prepared using known methods or as described in the experimental examples below, and analyzed by light microscopy or electron microscopy, for internalization as well as the location of the internalized antibody in the cell. For internalization in vitro, the cells can be incubated in tissue culture dishes in the presence or absence of the relevant antibodies added to the culture media and processed for microscopic analysis at desired time points. The presence of an internalized, labeled antibody in the cells can be directly visualized by microscopy or by autoradiography if radiolabeled antibody is used. Optionally, in microscopy, co-localization with a known polypeptide or other cellular component can be assessed; for example co-localization with endosomal/lysosomal marker LAMP-1 (CD107a) can provide information about the subcellular localization of the internalized antibody. Alternatively, in a quantitative biochemical assay, a population of cells comprising CD39-expressing cells are contacted in vitro or in vivo with a radiolabeled test antibody and the cells (if contacted in vivo, cells are then isolated after a suitable amount of time) are treated with a protease or subjected to an acid wash to remove un-internalized antibody on the cell surface. The cells are ground up and the amount of protease resistant, radioactive counts per minute (cpm) associated with each batch of cells is measured by passing the homogenate through a scintillation counter. Based on the known specific activity of the radiolabeled antibody, the number of antibody molecules internalized per cell can be deduced from the scintillation counts of the ground-up cells. Cells are “contacted” with antibody in vitro preferably in solution form such as by adding the cells to the cell culture media in the culture dish or flask and mixing the antibody well with the media to ensure uniform exposure of the cells to the antibody.

In one example, antibodies screening can comprise use of MALDI-TOF-based assays described herein to produce or test an antibody which binds and neutralizes the enzymatic activity of CD39 without dependent on induction of or increasing down-modulation of CD39 cell surface expression, can comprise the steps of:

(a) providing a plurality of binding molecules (e.g. antibodies) that bind a CD39 polypeptide,

(b) bringing each of the binding molecules into contact with CD39-expressing cells, optionally human B cells, optionally Ramos human lymphoma cells;

(c) assessing production of AMP by mass spectrometry, wherein a decrease in AMP generated indicates neutralization of ATPase activity;

(d) selecting a binding molecule (e.g. for further evaluation, for further processing, production of a quantity of, for use in treatment) that results in a decrease of AMP generated by at least 70%, optionally 80% or optionally 90%;

(e) optionally, where the molecule comprises an Fc domain, modifying the Fc domain (e.g. by introduction of one or more amino acid modifications) to reduce binding to human CD16, CD32a, CD32b and/or CD64 polypeptides;

(f) assessing the ability of the molecule to induce or increase intracellular internalization of CD39; and

(g) optionally, selecting a binding molecule (e.g. for further evaluation, for further processing, production of a quantity of, for use in treatment) that does not substantially induce or increase intracellular internalization of CD39.

In one example, antibodies screening can comprise use of mutant CD39 polypeptides to orient the selection of antibodies to the epitopes of antibody I-391. For example, a method of producing or testing an antibody which binds and neutralizes the enzymatic activity of CD39 without inducing or increasing down-modulation of CD39 cell surface expression, can comprise the steps of:

(a) providing a plurality of antibodies that bind a CD39 polypeptide,

(b) bringing each of said antibodies into contact with a mutant CD39 polypeptide comprising a mutation at 1, 2, 3 or 4 residues selected from the group consisting of Q96, N99, E143 and R147 (with reference to SEQ ID NO: 1), and assessing binding between the antibody and the mutant CD39 polypeptide, relative to binding between the antibody and a wild-type CD39 polypeptide comprising the amino acid sequence of SEQ ID NO: 1, and

(c) selecting an antibody (e.g. for further evaluation, for further processing, production of a quantity of, for use in treatment) that has reduced binding to the mutant CD39 polypeptide, relative to binding between the antibody and a wild-type CD39 polypeptide comprising the amino acid sequence of SEQ ID NO: 1; and optionally further:

(d) bringing each of the antibodies selected in step (c) into contact with CD39-expressing cells, optionally human B cells, optionally Ramos human lymphoma cells;

(e) assessing production of AMP by mass spectrometry, wherein a decrease in AMP generated indicates neutralization of ATPase activity; and

(f) selecting an antibody that results in a decrease of AMP generated by at least 70%, optionally 80% or optionally 90%.

### Epitopes on CD39

In one aspect, the antibodies bind an antigenic determinant present on CD39 expressed at the cell surface.

In one aspect, the antibodies bind substantially the same epitope as antibody I-391 and/or I-392. In one embodiment, the antibodies bind to an epitope of CD39 that at least partially overlaps with, or includes at least one residue in, the epitope bound by antibody I-391 and/or I-392. The residues bound by the antibody can be specified as being present on the surface of the of the CD39 polypeptide, e.g. in a CD39 polypeptide expressed on the surface of a cell.

Binding of anti-CD39 antibody to cells transfected with CD39 mutants can be measured and compared to the ability of anti-CD39 antibody to bind wild-type CD39 polypeptide (e.g., SEQ ID NO: 1). A reduction in binding between an anti-CD39 antibody and a mutant CD39 polypeptide (e.g. a mutant of Table 1) means that there is a reduction in binding affinity (e.g., as measured by known methods such FACS testing of cells expressing a particular mutant, or by Biacore testing of binding to mutant polypeptides) and/or a reduction in the total binding capacity of the anti-CD39 antibody (e.g., as evidenced by a decrease in Bmax in a plot of anti-CD39 antibody concentration versus polypeptide concentration). A significant reduction in binding indicates that the mutated residue is directly involved in binding to the anti-CD39 antibody or is in close proximity to the binding protein when the anti-CD39 antibody is bound to CD39.

In some embodiments, a significant reduction in binding means that the binding affinity and/or capacity between an anti-CD39 antibody and a mutant CD39 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type CD39 polypeptide. In certain embodiments, binding is reduced below detectable limits. In some embodiments, a significant reduction in binding is evidenced when binding of an anti-CD39 antibody to a mutant CD39 polypeptide is less than 50% (e.g., less than 45%, 40%, 35%, 30%, 25%, 20%, 15% or 10%) of the binding observed between the anti-CD39 antibody and a wild-type CD39 polypeptide.

In some embodiments, anti-CD39 antibodies are provided that exhibit significantly lower binding for a mutant CD39 polypeptide in which a residue in a segment comprising an amino acid residue bound by antibody I-391 or I-392 is substituted with a different amino acid.

In some embodiments, anti-CD39 antibodies (e.g. other than antibodies I-391 or I-392) are provided that bind the epitope on CD39 bound by antibodies I-391 or I-392.

In one aspect, the anti-CD39 antibodies have reduced binding to a CD39 polypeptide having a mutation at a residue selected from the group consisting of: Q96, N99, E143 and R147 (with reference to SEQ ID NO: 1); optionally, the mutant CD39 polypeptide has the mutations: Q96A, N99A, E143A and R147E.

In some embodiments, the antibodies do not exhibit significantly lower binding for a mutant CD39 polypeptide (e.g. mutants 7, 16 and 17 of Table 1) in which a residue in a segment comprising an amino acid residue bound by antibody A1 is substituted with a different amino acid, for example a mutant CD39 polypeptide comprising a substitution at one or more (or all of) residues A272, N274, 1276, R278, Q332, Q323, Q326, E330, N333, S335, Y336 and N345 (with reference to SEQ ID NO: 1). For example, in one embodiment, the epitope of the anti-CD39 antibodies does not comprise residues A272, N274, 1276, R278 (or A272, N274, 1276, R278, Q332, Q323, Q326, E330, N333, S335, Y336 and N345), and/or the anti-CD39 antibodies do not have reduced binding to a CD39 polypeptide having a mutation at a residue selected from the group consisting of: A272, N274, 1276, R278 (with reference to SEQ ID NO: 1); optionally, the mutant CD39 polypeptide has the mutations A272S, N274A, 1276S, R278A.

In one aspect, the anti-CD39 antibodies bind an epitope on CD39 comprising an amino acid residue (e.g. one, two, three or four of the residues) selected from the group consisting of Q96, N99, E143 and R147 (with reference to SEQ ID NO: 1).

In one aspect, the anti-CD39 antibodies bind an epitope on CD39 comprising an amino acid residue (e.g. at least one, two, three, four, five or six of the residues) selected from the group consisting of S92, K93, V95, Q96, K97, V98, N99, E100, L136, L137, E140, S141, L144, R147, D150, V151, R154, S294, D295, Y296, K298, P300, E306, 1308 and Q312 (with reference to SEQ ID NO: 1).

In one aspect, the anti-CD39 antibodies bind an epitope on CD39 comprising (a) an amino acid residue (e.g. at least one, two, three of the residues) in a first segment of residues of CD39 comprising residues S92, K93, V95, Q96, K97, V98, N99 and E100; (b) an amino acid residue (e.g. at least one, two, three of the residues) in a second segment of residues of CD39 comprising residues L136, L137, E140, S141, L144, R147, D150, V151, R154; and (c) an amino acid residue (e.g. at least one, two, three of the residues) in a third segment of residues of CD39 comprising residues S294, D295, Y296, K298, P300, E306, 1308 and Q312 (with reference to SEQ ID NO: 1).

In one aspect, the anti-CD39 antibodies bind an epitope on CD39 comprising an amino acid residue (e.g. at least one, two or three of the residues) selected from the group consisting of Q96, L137, and E140 (with reference to SEQ ID NO: 1).

In one aspect of any embodiment, the antibodies additionally bind to the glycan at position N292 of CD39.

### Exemplary Antibody Variable Region Sequences

An exemplary anti-CD39 VH and VL pair that can adapted according of the disclosure is that of antibody I-391, the amino acid sequence of the heavy chain variable region of which is listed below (SEQ ID NO: 6), and the amino acid sequence of the light chain variable region of which is listed below (SEQ ID NO: 7). Optionally, the VH and VL comprise (e.g. are modified to incorporate) human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 6. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 7. In one embodiment, an anti-CD39 antibody of the disclosure comprises a VH comprising the Kabat CDR1, CDR2 and/or CDR3 of the heavy chain variable region having the amino acid sequence of SEQ ID NO: 6 and a VL comprising a Kabat CDR1, CDR2 and/or CDR3 of the light chain variable region having the amino acid sequence of SEQ ID NO: 7.

I-391 VH
(SEQ ID NO: 6)
QIQLVQSGPELKKPGETVKISCKASGYTFRNYGMNWVKQAPGKGLKWMGW
INTYTGEPTYADDFKGRFAFSLATSASTAYLQISNLKNEDTATYFCARKA
YYGSNYYFDYWGQGTTLTVSS
I-391 VL
(SEQ ID NO: 7)
DIVMTQSHKFMSTSVGDRVSITCKASQDVSTAVAWYQQKPGQSPKLLIYS
ASYRYTGVPDRFTGSGSGTDFTFTISTVQAEDLAVYYCQQHYTTPPYTFG
GGTKLEIK

An anti-CD39 antibody may for example comprise: a HCDR1 comprising an amino acid sequence: NYGMN (SEQ ID NO: 25), or a sequence of at least 4, 5, 6, 7, 8, 9 or 10 contiguous amino acids thereof, optionally wherein one or more of these amino acids may be substituted by a different amino acid; a HCDR2 comprising an amino acid sequence: WINTYTGEPTYADDFKG (SEQ ID NO: 26), or a sequence of at least 4, 5, 6, 7, 8, 9 or 10 contiguous amino acids thereof, optionally wherein one or more of these amino acids may be substituted by a different amino acid; a FR3 comprising an amino acid sequence RFAFSL (SEQ ID NO: 27) or RFVFSL (SEQ ID NO: 28) at Kabat residues 66-70 optionally, a FR3 comprising an amino acid sequence LATS or LEAS (or, optionally LDTS or LETS) at Kabat residues 71 to 72b; a HCDR3 comprising an amino acid sequence: KAYYGSNYYFDY (SEQ ID NO: 29), or a sequence of at least 4, 5, 6, 7, 8, 9 or 10 contiguous amino acids thereof, optionally wherein one or more of these amino acids may be substituted by a different amino acid; a LCDR1 comprising an amino acid sequence: KASQDVSTAVA (SEQ ID NO: 30), or a sequence of at least 4, 5, 6, 7, 8, 9 or 10 contiguous amino acids thereof, optionally wherein one or more of these amino acids may be substituted by a different amino acid; a FR2 region comprising a tyrosine at Kabat residue 49; a LCDR2 region comprising an amino acid sequence: SASYRYT (SEQ ID NO: 31) or a sequence of at least 4, 5, 6, 7, 8, 9 or 10 contiguous amino acids thereof, optionally wherein one or more of these amino acids may be substituted by a different amino acid; and/or a LCDR3 region of I-391 comprising an amino acid sequence: QQHYTTPPYT (SEQ ID NO: 32), or a sequence of at least 4, 5, 6, 7, 8, 9 or 10 contiguous amino acids thereof, optionally wherein one or more of these amino acids may be deleted or substituted by a different amino acid. CDR positions may be according to Kabat numbering.

Another exemplary anti-CD39 VH and VL pair that can adapted according to the disclosure is that of antibody I-392, the amino acid sequence of the heavy chain variable region of which is listed below (SEQ ID NO: 8), and the amino acid sequence of the light chain variable region of which is listed below (SEQ ID NO: 9). Optionally, the VH and VL comprise (e.g. are modified to incorporate) human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 8. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 9. In one embodiment, an anti-CD39 antibody of the disclosure comprises a VH comprising the Kabat CDR1, CDR2 and/or CDR3 of the heavy chain variable region having the amino acid sequence of SEQ ID NO: 8 and a VL comprising a Kabat CDR1, CDR2 and/or CDR3 of the light chain variable region having the amino acid sequence of SEQ ID NO: 9.

I-392 VH
(SEQ ID NO: 8)
QIQLVQSGPEVKKPRETVKISCKASGYTFTHYGMNWVKQAPGKGLKWMGW
INTYTGEPTYADDFKGRFAFSLEASASTAYLQINNLKNEDTATYFCARRR
YEGNYVFYYFDYWGQGTTLTVSS
I-392 VL
(SEQ ID NO: 9)
DIQMTQSPASLSASVGETVTITCRASENIYSYFSWYQQKQGKSPQLLVYT
AKTLAEGVPSRFSGSGSGTQFSLKINSLQPEDFGSYYCQHHYVTPYTFGG
GTKLEIK

An anti-CD39 antibody may for example comprise: a HCDR1 comprising an amino acid sequence: HYGMN (SEQ ID NO: 33); a HCDR2 comprising an amino acid sequence: WINTYTGEPTYADDFKG (SEQ ID NO: 26); a FR3 comprising an amino acid sequence RFAFSL (SEQ ID NO: 27) or RFVFSL (SEQ ID NO: 28) at Kabat residues 66-70 optionally, a FR3 comprising an amino acid sequence LATS or LEAS (or, optionally LDTS or LETS) at Kabat residues 71 to 72b; a HCDR3 comprising an amino acid sequence: RRYEGNYVFYYFDY (SEQ ID NO: 34); a LCDR1 comprising an amino acid sequence: RASENIYSYFS (SEQ ID NO: 35); a FR2 region comprising a tyrosine at Kabat residue 49; a LCDR2 region comprising an amino acid sequence: TAKTLAE (SEQ ID NO: 36); and/or a LCDR3 region comprising an amino acid sequence: QHHYVTPYT (SEQ ID NO: 37). CDR positions may be according to Kabat numbering.

Another exemplary anti-CD39 VH and VL pair that can adapted according to the disclosure is that of the antibody having the amino acid sequence of the heavy chain variable region of which is listed below (SEQ ID NO: 10), and the amino acid sequence of the light chain variable region of which is listed below (SEQ ID NO: 11). Optionally, the VH and VL comprise (e.g. are modified to incorporate) human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 10. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 11. In one embodiment, an anti-CD39 antibody of the disclosure comprises a VH comprising the Kabat CDR1, CDR2 and/or CDR3 of the heavy chain variable region having the amino acid sequence of SEQ ID NO: 10 and a VL comprising a Kabat CDR1, CDR2 and/or CDR3 of the light chain variable region having the amino acid sequence of SEQ ID NO: 11.

VH
(SEQ ID NO: 10)
QVQLVQSGSELKKPGASVKISCKASGYTFTHYGMNWVRQAPGQGLEWMGW
INTYTGELTYADDFKGRFVFSLDTSVSTAYLQISSLKAEDTAVYYCARRA
YYRYDYVMDYWGQGTLVTVSS
VL
(SEQ ID NO: 11)
DIQMTQSPSSLSASVGDRVTITCKASHNVGTNVAWFQQKPGKAPKSLIYS
ASYRYSGVPSRFSGSGSGTDFTLTISSLQPEDFATYYCHQYNNYPYTFGQ
GTKLEIK

In one embodiment, an antibody comprises: a HCDR1 comprising an amino acid sequence: HYGMN (SEQ ID NO: 33); a HCDR2 comprising an amino acid sequence: WINTYTGELTYADDFKG (SEQ ID NO: 38); optionally, a FR3 comprising an amino acid sequence RFAFSL (SEQ ID NO: 27) or RFVFSL (SEQ ID NO: 28) at Kabat residues 66-70 optionally, a FR3 comprising an amino acid sequence LATS or LEAS (or, optionally LDTS or LETS) at Kabat residues 71 to 72b; a HCDR3 comprising an amino acid sequence: RAYYRYDYVMDY (SEQ ID NO: 39); a LCDR1 comprising an amino acid sequence: KASHNVGTNVA (SEQ ID NO: 40); a FR2 region comprising a tyrosine at Kabat residue 49; a LCDR2 region comprising an amino acid sequence: SASYRYS (SEQ ID NO: 41); and/or a LCDR3 region comprising an LCDR3 comprising an amino acid sequence: HQYNNYPYT (SEQ ID NO: 42). CDR positions may be according to Kabat numbering.

In any of the antibodies, the specified variable region, FR and/or CDR sequences may comprise one or more sequence modifications, e.g. a substitution (1, 2, 3, 4, 5, 6, 7, 8 or more sequence modifications). In one embodiment the substitution is a conservative modification.

In another aspect, the anti-CD39 compound comprises a VH domain having at least about 60%, 70% or 80% sequence identity, optionally at least about 85%, 90%, 95%, 97%, 98% or 99% identity, to the VH domain of SEQ ID NO: 6. In another aspect, the anti-CD39 antibody comprises a V_{L} domain having at least about 60%, 70% or 80% sequence identity, optionally at least about 85%, 90%, 95%, 97%, 98% or 99% identity, to the VL domain of SEQ ID NO: 7.

In another aspect, the anti-CD39 compound comprises a V_{H} domain having at least about 60%, 70% or 80% sequence identity, optionally at least about 85%, 90%, 95%, 97%, 98% or 99% identity, to the VH domain of SEQ ID NO: 8. In another aspect, the anti-CD39 antibody comprises a V_{L} domain having at least about 60%, 70% or 80% sequence identity, optionally at least about 85%, 90%, 95%, 97%, 98% or 99% identity, to the VL domain of SEQ ID NO: 9.

In another aspect, the anti-CD39 compound comprises a V_{H} domain having at least about 60%, 70% or 80% sequence identity, optionally at least about 85%, 90%, 95%, 97%, 98% or 99% identity, to the VH domain of SEQ ID NO: 10. In another aspect, the anti-CD39 antibody comprises a V_{L} domain having at least about 60%, 70% or 80% sequence identity, optionally at least about 85%, 90%, 95%, 97%, 98% or 99% identity, to the VL domain of SEQ ID NO: 11.

A range of other VH and VL domains, and antibodies comprising such, can be prepared based on the structural information provided herein. An exemplary binding molecule or antigen-binding fragment thereof capable of binding to and inhibiting the activity of CD39 may comprise a V_{H} and a V_{L}, wherein the V_{H} comprises:
optionally, a FR1 comprising a residue that is capable of contacting CD39, optionally wherein the residue is at Kabat position 30, optionally wherein the residue is a threonine,
a CDR1 comprising a residue at Kabat position 33, optionally at both positions 31 and 33, that is capable of contacting CD39;
a CDR2 comprising a residue at any 1, 2, 3, 4, 5 of 6 of Kabat positions 50, 52, 52a, 53, 54 and 56 that are capable of contacting CD39, optionally wherein the residue at position 53 comprises an aromatic ring, optionally tyrosine;
a FR3 comprising a residue at any 1, 2, 3, 4 of 5 of Kabat positions 67, 68, 69, 70 and 71 capable of contacting with CD39, optionally wherein FR3 further comprises a residue at any 1, 2 of 3 of Kabat positions 72, 72a and 72b capable of contacting with CD39, and
a CDR3 comprising a residue at any 1, 2 or more of Kabat positions 100, 100b, 100c, 100d, 100e and/or 100f (to the extent a residue is present at the particular position) that is capable of contacting CD39, wherein the residue(s) comprise an aromatic ring, optionally tyrosine. Optionally, the CDR3 comprises a residue at any 1, 2 or more of Kabat positions 100, 100b, 100c, 100d, 100e and/or 100f (to the extent a residue is present at the particular position) that is capable of contacting the V_{L}, wherein the residue comprises an aromatic ring.

Heavy Chain CDR1 (and FR1)

An exemplary CDR1 (optionally together with one or more residues in the FR1) binds the N-terminal domain of CD39. A V_{H} can for example have CD39 contact residues at Kabat positions 30 (within the Kabat FR1, adjacent to the CDR1); residue 30 may contact residue Q96 of CD39. In one embodiment, a V_{H} can for example have CD39 contact residues at Kabat position 31 (within the Kabat CDR1). In one embodiment, a V_{H} can for example have CD39 contact residues at Kabat position 32 (within the Kabat CDR1). In one embodiment, a V_{H} can for example have CD39 contact residues at Kabat position 33 (within the Kabat CDR1).

In one embodiment, a V_{H} comprises a CDR1 wherein the residues at Kabat position 31, 32 and 33 have the formula X₁ X₂ X₃, wherein X₁ represents any amino acid, optionally a histidine or asparagine, or optionally a conservative substitution thereof, X₂ represents any amino acid, optionally an aromatic residue, optionally a tyrosine or a conservative substitution thereof, or optionally an amino acid other than proline or glycine, and X₃ represents glycine. Optionally, the residues at Kabat positions 32, 34 and/or 35 are identical to the corresponding residue in the human acceptor sequence of the V_{H}.

In one embodiment, the V_{H} comprises a CDR1 wherein the residues at Kabat position 31 to 35 have the formula X₁ X₂ X₃ X₄ X₅ (SEQ ID NO: 43), wherein X₁ represents histidine or asparagine, or a conservative substitution thereof, X₂ represents any amino acid, optionally an aromatic residue, optionally tyrosine, or a conservative substitution thereof, X₃ represents glycine, or a conservative substitution thereof, X₄ represents any amino acid, optionally a methionine, or a conservative substitution thereof, and X₅ represents any amino acid, optionally an asparagine, or a conservative substitution thereof. In one embodiment, the CDR1 comprises an amino acid sequence HYGMN (SEQ ID NO: 33), optionally comprising one or two amino acid substitutions. In one embodiment, the Kabat positions 31-35 have an amino acid sequence that differs (e.g. by one or more amino acid residues) from the amino acid sequence HYGMN (SEQ ID NO: 33).

Heavy Chain CDR2-FR3 Segment

A CDR2 (e.g. according to Kabat) can be capable of contacting the N-terminal domain of CD39 (e.g. via residues within the segment of Kabat positions 50-56), and can further comprise residues capable of contacting the C-terminal domain of CD39 (together with residues of the Kabat FR3 domain, e.g. within the segment of Kabat positions 59-71 or optionally 59-72b).

For example, a CDR2 can have a residue at Kabat position 50 capable of contacting CD39. In one embodiment, a CDR2 can have a residue at Kabat position 52 capable of contacting CD39. In one embodiment, a CDR2 can have a residue at Kabat position 52a capable of contacting CD39. In one embodiment, a CDR2 can have a residue at Kabat position 53 capable of contacting CD39, optionally an aromatic residue. In one embodiment, CDR2 can have a residue at Kabat position 54 capable of contacting CD39. In one embodiment, a CDR2 can have a residue at Kabat position 56 capable of contacting CD39.

In one embodiment, the V_{H} comprises a CDR2 wherein the residues at Kabat position 50-56 have the formula X₁ X₂ X₃ X₄ X₅ X₆ X₇ X₈ (SEQ ID NO: 13), wherein X₁ represents tryptophan, X₂ represents any amino acid, optionally an isoleucine, X₃ represents asparagine or optionally glutamine, X₄ represents threonine, X₅ represents any amino acid, optionally tyrosine or optionally phenylalanine, X₆ represents any amino acid, optionally threonine, optionally serine, optionally asparagine, alanine or glycine, optionally residues other than large or hydrophobic resides, X₇ represents any amino acid, optionally glycine, optionally alanine, serine, threonine, asparagine or glutamine, optionally residues other than aspartic acid or glutamic acid, optionally residues other than lysine or arginine, and X₈ represents glutamic acid, optionally aspartic acid. In one embodiment, the Kabat positions 50-56 have an amino acid sequence WINTYTGE (SEQ ID NO: 44), optionally comprising one or two amino acid substitutions. In one embodiment, the Kabat positions 50-65 have an amino acid sequence WINTYTGEPTYADDFKG (SEQ ID NO: 26) or WINTYTGELTYADDFKG (SEQ ID NO: 38). In another embodiment, the Kabat positions 50-65 have an amino acid sequence that differs (e.g. by one or more amino acid residues) from the amino acid sequence WINTYTGEPTYADDFKG (SEQ ID NO: 26) and/or WINTYTGELTYADDFKG (SEQ ID NO: 38).

In one embodiment, the V_{H} FR3 (according to Kabat) comprises residues that are capable of contacting amino acid residues in the C-terminal domain of CD39, optionally further wherein residues within Kabat positions 59-71 contact the glycan at residue N292 of CD39. In one embodiment, the V_{H} Kabat FR3 comprises residues at Kabat positions 67, 68, 69, 70 and/or 71, and optionally further at residue 72, 72a and/or 72b that are capable of contacting the C-terminal domain of CD39, e.g. including amino acid resides in CD39 and/or the glycan at N292 of the CD39 polypeptide.

In one embodiment, the Kabat positions 59-71 have an amino acid sequence YADDFKGRFAFSL (SEQ ID NO: 45) or YADDFKGRFVFSL (SEQ ID NO: 46), optionally comprising one or two amino acid substitutions, or an amino acid sequence that differs (e.g. by one or more amino acid residues) from the amino acid sequence YADDFKGRFAFSL (SEQ ID NO: 45) or YADDFKGRFVFSL (SEQ ID NO: 46).

For example, a CDR2 can have a residue at Kabat position 67 capable of contacting the C-terminal domain of CD39. In one embodiment, a CDR2 can have a residue at Kabat position 68 capable of contacting CD39. In one embodiment, a CDR2 can have a residue at Kabat position 69 capable of contacting CD39. In one embodiment, a CDR2 can have a residue at Kabat position 70 capable of contacting CD39. In one embodiment, a CDR2 can have a residue at Kabat position 71 capable of contacting CD39.

In one embodiment, the V_{H} FR3 (e.g. the N-terminal segment of the Kabat FR3) comprises residues at Kabat position 66-71 having the formula X₁ X₂ X₃ X₄ X₅ X₆ (SEQ ID NO: 47), wherein X₁ represents any amino acid, optionally arginine, X₂ represents phenylalanine or another hydrophobic residue capable of maintaining the beta-strand position and V_{H} domain structure integrity, X₃ represents alanine or valine, or optionally leucine, optionally a hydrophobic residue, X₄ represents phenylalanine or another hydrophobic residue capable of maintaining the beta-strand position and V_{H} domain structure integrity and X₅ represents serine, and X₆ represents any amino acid, optionally leucine, optionally alanine, valine or threonine.

In one embodiment, the V_{H} FR3 (according to Kabat) comprises residues at Kabat positions 72, 72a and 72b having the formula X₁ X₂ X₃, wherein X₁ represents aspartic acid, glutamic acid or alanine, X₂ represents any amino acid, optionally alanine or threonine, or a conservative substitution thereof, and X₃ represents serine, optionally alanine, or a conservative substitution thereof.

In one embodiment, the V_{H} comprises the FR3 signature sequence FVFSL (SEQ ID NO: 59) at Kabat positions 67-71. In one embodiment, the V_{H} comprises a human acceptor framework or portion thereof (e.g. an FR3 domain) that naturally comprises the amino acid sequence FVFSL (SEQ ID NO: 59) at Kabat positions 67-71. In another embodiment, the V_{H} comprises a human acceptor framework or portion thereof (e.g. an FR3 domain) that is comprises one or more amino acid modifications (e.g., one or more substitution(s) at Kabat positions 67-71) and comprises the amino acid sequence FVFSL (SEQ ID NO: 59) at Kabat positions 67-71.

Heavy Chain CDR3

In one embodiment, the V_{H} comprises a CDR3 (e.g. according to Kabat) capable of contacting the N-terminal of CD39, optionally capable of contacting the N-terminal domain of CD39 and the V_{L}, optionally wherein the CDR3 comprises an aromatic residue (e.g. a tyrosine, a phenylalanine) that is capable of binding an amino acid residue in the N-terminal domain of CD39 and a second aromatic amino acid residue (e.g. a tyrosine, a phenylalanine) that is capable of contacting an amino acid residue in the V_{L}.

An exemplary Kabat CDR3 may comprise a sequence of amino residues having the formula X₁ X₂ X₃ X₄ X₅ (SEQ ID NO: 15), wherein any three or more of X₁, X₂, X₃, X₄ and X₅ represent an aromatic amino acid. Optionally, at least three of the aromatic residues are tyrosines. Optionally at least two aromatic residues are tyrosines and at least one aromatic residue is a phenylalanine.

For example, a CDR3 can have a residue at Kabat position 95 capable of contacting CD39. In one embodiment, a CDR3 can have a residue at Kabat position 99 capable of contacting CD39. In one embodiment, a CDR3 can have a residue at Kabat position 100b capable of contacting CD39. In one embodiment, a CDR3 can have a residue at Kabat position 100d capable of contacting CD39, optionally an aromatic residue. In one embodiment, CDR3 can have a residue at Kabat position 100f capable of contacting CD39.

Optionally the residues at Kabat position 95-102 have the formula X₁ X₂ X₃ X₄ X₅ X₆ X₇ X₈ X₉ X₁₀ X₉ X₁₀ X₁₁ X₁₂ X₁₃ X₁₄ (SEQ ID NO: 16), wherein:
X₁ represents arginine or lysine, or optionally a conservative substitution thereof,
X₂ represents any amino acid, optionally arginine, optionally lysine or alanine, or optionally a conservative substitution thereof,
X₃ represents any amino acid residue, optionally a residue comprising an aromatic ring, optionally a tyrosine,
X₄ represents any amino acid, optionally glutamic acid or tyrosine, or optionally a conservative substitution thereof, or amino acid residues other than proline or glycine,
X₅ represents glycine, optionally arginine, or optionally a conservative substitution thereof,
X₆ represents any amino acid, optionally asparagine, serine or tyrosine, or optionally a conservative substitution thereof,
X₇ represents any amino acid, optionally tyrosine, asparagine or aspartic acid, or optionally a conservative substitution thereof, optionally an amino acid residue other than proline or glycine,
X₈ represents valine or optionally alanine, isoleucine or leucine, optionally an aromatic amino acid, optionally tyrosine,
X₉ represents any amino acid, optionally an aromatic amino acid, optionally phenylalanine, optionally tyrosine, optionally valine or a conservative substitution thereof,
X₁₀ represents tyrosine, optionally phenylalanine, optionally methionine, or optionally a conservative substitution thereof,
X₁₁ is absent or represents any amino acid, optionally tyrosine, optionally phenylalanine, optionally phenylalanine, or optionally a conservative substitution thereof, optionally an amino acid residue other than P, G, E or D, or other than a small hydrophobic residue (e.g. T, S),
X₁₂ is absent or represents any amino acid, optionally phenylalanine, or optionally a conservative substitution thereof,
X₁₃ represents any amino acid, optionally aspartic acid, or optionally a conservative substitution thereof, optionally a serine, optionally a threonine, optionally a glutamic acid, optionally an asparagine, optionally a residue other than a large and hydrophobic residue, and
X₁₄ represents any amino acid, optionally tyrosine or optionally a conservative substitution thereof, optionally an aromatic amino acid, optionally a non-aromatic amino acid.

In one embodiment, the Kabat positions 95-100f are present and have an amino acid sequence RRYEGNYVFYYF (SEQ ID NO: 48). In one embodiment, the Kabat positions 95-100d are present and have an amino acid sequence KAYYGSNYYF (SEQ ID NO: 49) or RAYYRYDYVM (SEQ ID NO: 50), optionally comprising one, two, three, four or five amino acid substitutions. In one embodiment, the residue at Kabat position 101 is an aspartic acid (D). In another embodiment, the Kabat positions 95-102 have an amino acid sequence that differs (e.g. by one or more amino acid residues) from the amino acid sequence

RRYEGNYVFYYFDY (SEQ ID NO: 48), KAYYGSNYYFDY (SEQ ID NO: 49) and/or RAYYRYDYVMDY (SEQ ID NO: 50).

An exemplary V_{H} can comprise:
(a) a CDR1 (e.g. according to Kabat) capable of contacting the N-terminal domain of CD39, optionally wherein the residues at Kabat position 31, 32 and 33 have the formula X₁ X₂ X₃, wherein X₁ represents any amino acid, optionally a histidine or asparagine, or optionally a conservative substitution thereof, X₂ represents any amino acid, optionally an aromatic residue, optionally a tyrosine or a conservative substitution thereof, or optionally an amino acid other than proline or glycine, and X₃ represents glycine;
(b) a CDR2 (e.g. according to Kabat) capable of contacting the C-terminal domain of CD39, optionally wherein the residues at Kabat position 50-56 have the formula X₁ X₂ X₃ X₄ X₅ X₆ X₇ X₈ (SEQ ID NO: 13), wherein X₁ represents tryptophan, X₂ represents any amino acid, optionally an isoleucine, X₃ represents asparagine or optionally glutamine, X₄ represents threonine, X₅ represents any amino acid, optionally tyrosine or optionally phenylalanine, X₆ represents any amino acid, optionally threonine, optionally serine, optionally asparagine, alanine or glycine, optionally residues other than large or hydrophobic resides, X₇ represents any amino acid, optionally glycine, optionally alanine, serine, threonine, asparagine or glutamine, optionally residues other than aspartic acid or glutamic acid, optionally residues other than lysine or arginine, and X₈ represents glutamic acid, optionally aspartic acid;
(c) optionally, an FR3 comprising residues at Kabat position 66-71 having the formula X₁ X₂ X₃ X₄ X₅ X₆ (SEQ ID NO: 47), wherein X₁ represents any amino acid, optionally arginine, X₂ represents phenylalanine or another hydrophobic residue capable of maintaining the beta-strand position and V_{H} domain structure integrity, X₃ represents alanine or valine, or optionally leucine, optionally a hydrophobic residue, X₄ represents phenylalanine or another hydrophobic residue capable of maintaining the beta-strand position and V_{H} domain structure integrity and X₅ represents serine, optionally further wherein and X₆ represents any amino acid, optionally leucine, optionally alanine, valine or threonine; and
(d) a CDR3 (e.g. according to Kabat) capable of contacting the N-terminal of CD39, optionally capable of contacting the N-terminal domain of CD39 and the V_{L}, optionally wherein the CDR3 comprises an aromatic residue (e.g. a tyrosine, a phenylalanine) that is capable of binding an amino acid residue in the N-terminal domain of CD39 and a second aromatic amino acid residue (e.g. a tyrosine, a phenylalanine) that is capable of contacting an amino acid residue in the V_{L}, optionally wherein the residues at Kabat position 95-102 have the formula X₁ X₂ X₃ X₄ X₅ X₆ X₇ X₈ X₉ X₁₀ X₉ X₁₀ X₁₁ X₁₂ X₁₃ X₁₄ (SEQ ID NO: 16), wherein
X₁ represents arginine or lysine, or optionally a conservative substitution thereof,
X₂ represents any amino acid, optionally arginine, optionally lysine or alanine, or optionally a conservative substitution thereof,
X₃ represents any amino acid residue, optionally a residue comprising an aromatic ring, optionally a tyrosine,
X₄ represents any amino acid, optionally glutamic acid or tyrosine, or optionally a conservative substitution thereof, or amino acid residues other than proline or glycine,
X₅ represents glycine, optionally arginine, or optionally a conservative substitution thereof,
X₆ represents any amino acid, optionally asparagine, serine or tyrosine, or optionally a conservative substitution thereof,
X₇ represents any amino acid, optionally tyrosine, asparagine or aspartic acid, or optionally a conservative substitution thereof, optionally amino acid residues other than proline or glycine,
X₈ represents valine or optionally alanine, isoleucine or leucine, optionally an aromatic amino acid, optionally tyrosine,
X₉ represents any amino acid, optionally an aromatic amino acid, optionally phenylalanine, optionally tyrosine, optionally valine or a conservative substitution thereof,
X₁₀ represents tyrosine, optionally phenylalanine, optionally methionine, or optionally a conservative substitution thereof,
X₁₁ is absent or represents any amino acid, optionally tyrosine, optionally phenylalanine, optionally phenylalanine, or optionally a conservative substitution thereof, optionally an amino acid residue other than P, G, E or D, or other than a small hydrophobic residues (e.g. T, S),
X₁₂ is absent or represents any amino acid, optionally phenylalanine, or optionally a conservative substitution thereof,
X₁₃ represents any amino acid, optionally aspartic acid, or optionally a conservative substitution thereof, optionally a serine, optionally a threonine, optionally a glutamic acid, optionally an asparagine, optionally a residue other than a large and hydrophobic residue, and
X₁₄ represents any amino acid, optionally tyrosine or optionally a conservative substitution thereof, optionally an aromatic amino acid, optionally a non-aromatic amino acid. Optionally, the residue at Kabat position 30 (FR1) is a threonine.

Another exemplary V_{H} can comprise:
(a) a CDR1 (e.g. according to Kabat) capable of contacting the N-terminal domain of CD39, optionally wherein the residues at Kabat position 31, 32 and 33 have the formula X₁ X₂ X₃, wherein X₁ represents any amino acid, optionally a histidine, optionally a conservative substitution thereof, X₂ represents any amino acid, optionally an aromatic residue, optionally a tyrosine, or a conservative substitution thereof, or optionally an amino acid residue other that proline or glycine, and X₃ represents glycine;
(b) a CDR2 (e.g. according to Kabat) capable of contacting the C-terminal domain of CD39, optionally wherein the residues at Kabat position 50-56 having the formula X₁ X₂ X₃ X₄ X₅ X₆ X₇ X₈ (SEQ ID NO: 13), wherein X₁ represents tryptophan, X₂ represents any amino acid, optionally an isoleucine, X₃ represents asparagine or optionally glutamine, X₄ represents threonine, X₅ represents any amino acid, optionally tyrosine or optionally phenylalanine, X₆ represents any amino acid, optionally threonine, optionally serine, optionally asparagine, alanine or glycine, optionally a residue other than large or hydrophobic resides, X₇ represents any amino acid, optionally glycine, optionally alanine, serine, threonine, asparagine or glutamine, optionally a residue other than aspartic acid or glutamic acid, optionally a residue other than lysine or arginine, and X₈ represents glutamic acid, optionally aspartic acid;
(c) optionally, an FR3 comprising residues at Kabat position 66-71 having the formula X₁ X₂ X₃ X₄ X₅ X₆ (SEQ ID NO: 47), wherein X₁ represents any amino acid, X₂ represents phenylalanine or another hydrophobic residue capable of maintaining the beta-strand position and V_{H} domain structure integrity, X₃ represents alanine or valine, or optionally leucine, optionally a hydrophobic residue, X₄ represents phenylalanine or another hydrophobic residue capable of maintaining the beta-strand position and V_{H} domain structure integrity and X₅ represents serine, optionally further wherein and X₆ represents any amino acid, optionally leucine, optionally alanine, valine or threonine; and
(d) a CDR3 (e.g. according to Kabat) capable of contacting the N-terminal of CD39, optionally capable of contacting the N-terminal domain of CD39 and the V_{L}, optionally wherein the CDR3 comprises an aromatic residue (e.g. a tyrosine, a phenylalanine) that is capable of binding an amino acid residue in the N-terminal domain of CD39 and a second aromatic amino acid residue (e.g. a tyrosine, a phenylalanine) that is capable of contacting an amino acid residue in the V_{L}, optionally wherein the residues at Kabat position 95-102 have the formula X₁ X₂ X₃
   X₄ X₅ X₆ X₇ X₈ X₉ X₁₀ X₉ X₁₀ X₁₁ X₁₂ X₁₃ X₁₄ (SEQ ID NO: 16), wherein
   X₁ represents arginine or lysine, or optionally a conservative substitution thereof, X₂ represents any amino acid, optionally arginine, optionally lysine, or optionally a conservative substitution thereof,
   X₃ represents any amino acid residue, optionally a residue comprising an aromatic ring, optionally a tyrosine,
   X₄ represents any amino acid, optionally glutamic acid, or optionally a conservative substitution thereof, or an amino acid residue other than proline or glycine,
   X₅ represents glycine, or optionally a conservative substitution thereof,
   X₆ represents any amino acid, optionally asparagine, or optionally a conservative substitution thereof,
   X₇ represents any amino acid, optionally tyrosine, asparagine or aspartic acid, or optionally a conservative substitution thereof, optionally an amino acid residue other than proline or glycine,
   X₈ represents valine or optionally alanine, isoleucine or leucine,
   X₉ represents any amino acid, optionally an aromatic amino acid, optionally phenylalanine, optionally tyrosine,
   X₁₀ represents tyrosine, optionally phenylalanine,
   X₁₁ or represents any amino acid, optionally tyrosine, optionally phenylalanine, optionally phenylalanine, or optionally a conservative substitution thereof, optionally an amino acid residue other than P, G, E or D, or other than a small hydrophobic residue (e.g. T, S),
   X₁₂ or represents any amino acid, optionally phenylalanine, or optionally a conservative substitution thereof,
   X₁₃ represents any amino acid, optionally aspartic acid, or optionally a conservative substitution thereof, optionally a serine, optionally a threonine, optionally a glutamic acid, optionally an asparagine, optionally a residue other than a large and hydrophobic residue, and
X₁₄ represents any amino acid, optionally tyrosine or optionally a conservative substitution thereof, optionally an aromatic amino acid, optionally a non-aromatic amino acid.

An exemplary V_{L} can comprise:
a CDR1 comprising a residue at Kabat positions 31, 32, 33 and/or 34 capable of contacting the CDR3 of the V_{H};
a FR2 comprising an aromatic residue, optionally a tyrosine, at Kabat position 49; and
a CDR3 comprising a residue at Kabat positions 89 and/or 91 capable of contacting the CDR3 of the V_{H}.

Light Chain CDR1

In one embodiment, the V_{L} comprises a CDR1 wherein the residues at Kabat positions 31-34 have the formula X₁ X₂ X₃ X₄, wherein X₁ represents a serine or a threonine, or a conservative substitution thereof, X₂ represents a tyrosine, alanine or asparagine, or a conservative substitution thereof, X₃ represents phenylalanine or valine, and X₄ represents serine or alanine, or a conservative substitution thereof.

In one embodiment, the V_{L} comprises a CDR1 wherein the residues at Kabat positions 31-34 have the formula X₁ X₂ X₃ X₄, wherein X₁ represents a threonine or a conservative substitution thereof, X₂ represents alanine or asparagine, or a conservative substitution thereof, X₃ represents valine or a conservative substitution thereof, and X₄ represents alanine or a conservative substitution thereof.

In one embodiment, the V_{L} comprises a CDR1 wherein the residues at Kabat positions 31-34 have the formula SYX₁X₂, wherein S is a serine, Y is a tyrosine, X, represents a hydrophobic residue (e.g. a phenylalanine, isoleucine, valine), and X₂ represents any amino acid, optionally histidine, serine or alanine, or a conservative substitution thereof.

a CDR1 wherein the residues at Kabat position 31, 32, 33 and 34 have (a) the formula TX₁VA, wherein T is a threonine, V is a valine, A is an alanine, and X₁ represents alanine or asparagine, or a conservative substitution thereof.

In one embodiment, the V_{L} comprises a CDR1 wherein the residues at Kabat position 24 to 34 have the formula X₁ X₂ X₃ X₄ X₅ X₆ X₇ X₅ X₉ X₁₀ X₉ X₁₀ X₁₁ (SEQ ID NO: 17), wherein
X₁ represents any amino acid, optionally arginine or lysine, or a conservative substitution thereof,
X₂ represents any amino acid, optionally alanine, or a conservative substitution thereof,
X₃ represents any amino acid, optionally serine, or a conservative substitution thereof,
X₄ represents any amino acid, optionally glutamic acid or histidine, or a conservative substitution thereof,
X₅ represents any amino acid, optionally asparagine or aspartic acid, or a conservative substitution thereof,
X₆ represents any amino acid, optionally isoleucine or valine, or a conservative substitution thereof,
X₇ represents any amino acid, optionally tyrosine or glycine, or a conservative substitution thereof,
X₈ represents serine or threonine, or a conservative substitution thereof,
X₉ represents tyrosine, alanine or asparagine, or a conservative substitution thereof,
X₁₀ represents phenylalanine or valine, or a conservative substitution thereof, and
X₁₁ represents serine or alanine, or a conservative substitution thereof.

Optionally, the residues at Kabat positions 24 to 30 are at least 60%, 70%, 80%, 90% identical, or 100% identical, to the corresponding residue in the human acceptor sequence of the V_{L}. In one embodiment, the Kabat positions 24 to 30 have an amino acid sequence RASENIY (SEQ ID NO: 51), KASQDVS (SEQ ID NO: 52) or KASHNVG (SEQ ID NO: 53). In one embodiment, the Kabat positions 31-34 have an amino acid sequence SYFS, TAVA or TNVA. In one embodiment, the Kabat positions 24-34 have an amino acid sequence RASENIYSYFS (SEQ ID NO: 35), KASQDVSTAVA (SEQ ID NO: 30) or KASHNVGTNVA (SEQ ID NO: 40), optionally comprising one, two or three amino acid substitutions. In another embodiment, the Kabat positions 24-34 have an amino acid sequence that differs (e.g. by one or more amino acid residues) from the amino acid sequence RASENIYSYFS (SEQ ID NO: 35), KASQDVSTAVA (SEQ ID NO: 30) and/or KASHNVGTNVA (SEQ ID NO: 40).

Light Chain CDR2

In one embodiment, the V_{L} comprises a CDR2 that comprises a Kabat FR residue. In one embodiment, the residue at Kabat position 49 is an aromatic amino acid, optionally a tyrosine. In one embodiment, the residue at Kabat position 50 is a serine or threonine or a conservative substitution thereof.

In one embodiment, the V_{L} comprises a CDR2 wherein the residues at Kabat position 49-56 have the formula X₁ X₂ X₃ X₄ X₅ X₆ X₇ X₅ (SEQ ID NO: 54), wherein
X₁ represents an aromatic amino acid residue, optionally a tyrosine,
X₂ represents threonine or serine, or a conservative substitution thereof,
X₃ represents any amino acid, optionally alanine, or a conservative substitution thereof,
X₄ represents any amino acid, optionally lysine or serine, or a conservative substitution thereof,
X₅ represents any amino acid, optionally threonine or tyrosine, or a conservative substitution thereof,
X₆ represents any amino acid, optionally leucine or arginine, or a conservative substitution thereof,
X₇ represents any amino acid, optionally alanine or tyrosine, or a conservative substitution thereof, and
X₈ represents any amino acid, optionally glutamic acid, threonine or a conservative substitution thereof.

In one embodiment, the V_{L} comprises a CDR2 wherein the residues at Kabat position 50-56 have the formula X₁ X₂ X₃ X₄ X₅ X₆ X₇ (SEQ ID NO: 19), wherein:
X₁ represents serine, or a conservative substitution thereof,
X₂ represents alanine, or a conservative substitution thereof,
X₃ represents serine, or a conservative substitution thereof,
X₄ represents tyrosine, or a conservative substitution thereof,
X₅ represents arginine, or a conservative substitution thereof,
X₆ represents tyrosine, or a conservative substitution thereof, and
X₇ represents threonine or a conservative substitution thereof. Optionally, the V_{L} further comprises an aromatic residue, e.g. a tyrosine, at Kabat residue 49.

Optionally, the residues at Kabat positions 51 to 56 are at least 60%, 70%, 80%, 90% identical, or 100% identical, to the corresponding residue in the human acceptor sequence of the V_{L}. In one embodiment, the Kabat positions 49 to 51 have an amino acid sequence YTA or YSA. In one embodiment, the Kabat positions 49 to 56 have an amino acid sequence YTAKTLAE (SEQ ID NO: 55), YSASYRYT (SEQ ID NO: 56) or YSASYRYS (SEQ ID NO: 57). In another embodiment, the Kabat positions 50-56 have an amino acid sequence that differs (e.g. by one or more amino acid residues) from the amino acid sequence TAKTLAE (SEQ ID NO: 36), YSASYRYT (SEQ ID NO: 31) and/or YSASYRYS (SEQ ID NO: 41).

Light Chain CDR3

In one embodiment, the V_{L} comprises a CDR3 wherein the residues at Kabat position 89-91 have the formula X₁ X₂ X₃, wherein X₁ represents any amino acid, optionally a glutamine or histidine, or a conservative substitution thereof, X₂ represents any amino acid, optionally a glutamine or histidine, or a conservative substitution thereof, and X₃ represents tyrosine or histidine, or a conservative substitution thereof.

In one embodiment, the V_{L} comprises a CDR3 wherein the residues at Kabat position 89 is a glutamine or histidine, or a conservative substitution thereof, the residue at position 91 is a tyrosine or histidine, or a conservative substitution thereof, the residue at position 95 is a proline, or a conservative substitution thereof, and the residue at position 96 is a tyrosine, or a conservative substitution thereof.

In one embodiment, the V_{L} comprises a CDR3 wherein the residues at Kabat position 89-97 have the formula X₁ X₂ X₃ X₄ X₅ X₆ X₇ X₈ X₉X₁₀ (SEQ ID NO: 20), wherein
X₁ represents glutamine or histidine, or a conservative substitution thereof,
X₂ represents any amino acid, optionally glutamine or histidine, or a conservative substitution thereof,
X₃ represents tyrosine or histidine, or a conservative substitution thereof,
X₄ represents any amino acid, optionally tyrosine or asparagine, or a conservative substitution thereof,
X₅ represents any amino acid, optionally valine or asparagine, or a conservative substitution thereof,
X₆ represents any amino acid, optionally threonine or tyrosine, or a conservative substitution thereof,
X₇ represents any amino acid, optionally proline, or a conservative substitution thereof,
X₈ is absent or represents any one or more amino acids, optionally a proline, or a conservative substitution thereof,
X₉ represents any amino acid, optionally tyrosine, or a conservative substitution thereof, and
X₁₀ represents any amino acid, optionally threonine, or a conservative substitution thereof.

In one embodiment, the V_{L} comprises a CDR3 wherein the residues at Kabat position 89-97 have the formula QX₁HX₂X₃TPYT (SEQ ID NO: 58), wherein
X₁ represents any amino acid, optionally glutamine or histidine, or a conservative substitution thereof,
X₂ represents any amino acid, and
X₃ represents any amino acid.

In one embodiment, the Kabat positions 89-97 have an amino acid sequence QHHYVTPYT (SEQ ID NO: 37), QQHYTTPPYT (SEQ ID NO: 32) or HQYNNYPYT (SEQ ID NO: 42). In another embodiment, the Kabat positions 89-97 have an amino acid sequence that differs (e.g. by one or more amino acid residues) from the amino acid sequence QHHYVTPYT (SEQ ID NO: 37), QQHYTTPPYT (SEQ ID NO: 32) and/or HQYNNYPYT (SEQ ID NO: 42).

In one embodiment, the V_{L} comprises:
a CDR1 wherein the residues at Kabat position 31, 32, 33 and 34 have the formula X₁ X₂ X₃ X₄, wherein X₁ represents a threonine or a conservative substitution thereof, X₂ represents alanine or asparagine, or a conservative substitution thereof, X₃ represents valine or a conservative substitution thereof, and X₄ represents alanine or a conservative substitution thereof;
a FR2 comprising an aromatic residue, optionally a tyrosine, at Kabat position 49;
a CDR2 wherein the residue at Kabat position 50 is a serine or threonine or a conservative substitution thereof; and
a CDR3 wherein the residues at Kabat position 89 is a glutamine or histidine, or a conservative substitution thereof, the residue at position 91 is a tyrosine or histidine, or a conservative substitution thereof, optionally wherein the residue at position 95 is a proline, or a conservative substitution thereof, optionally wherein the residue at position 96 is a tyrosine, or a conservative substitution thereof.

Fragments and derivatives of antibodies (which are encompassed by the term “antibody” or “antibodies” as used in this application, unless otherwise stated or clearly contradicted by context) can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific (e.g. bispecific) antibodies formed from antibody fragments. Included, inter alia, are a nanobody, domain antibody, single domain antibody or a “dAb”.

In certain embodiments, the DNA of a hybridoma producing an antibody, can be modified prior to insertion into an expression vector, for example, by substituting the coding sequence for human heavy- and light-chain constant domains in place of the homologous non-human sequences (e.g., Morrison et al., PNAS pp. 6851 (1984)), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody.

Optionally an antibody is humanized. “Humanized” forms of antibodies are specific chimeric immunoglobulins, immunoglobulin chains or fragments thereof (such as Fv, Fab, Fab′, F (ab′) 2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.) Methods for humanizing the antibodies are well known in the art.

The choice of human variable domains, both light and heavy, to be used in making the humanized antibodies is very important to reduce antigenicity. According to the so-called “best-fit” method, the sequence of the variable domain of an antibody is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, 1987, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et al., J. Immunol., 151, p. 2623 (1993)).

It is further important that antibodies be humanized with retention of high affinity for CD39 and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence. In this way, FR residues can be selected and combined from the consensus and import sequences so that the desired antibody characteristic is achieved.

Another method of making “humanized” monoclonal antibodies is to use a XenoMouse (Abgenix, Fremont, Calif.) as the mouse used for immunization. A XenoMouse is a murine host according that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference.

Human antibodies may also be produced according to various other techniques, such as by using, for immunization, other transgenic animals that have been engineered to express a human antibody repertoire (Jakobovitz et al., Nature 362 (1993) 255), or by selection of antibody repertoires using phage display methods. Such techniques are known to the skilled person and can be implemented starting from monoclonal antibodies as disclosed in the present application.

### Antibody Formulations

An anti-CD39 antibody can be incorporated in a pharmaceutical formulation comprising in a concentration from 1 mg/ml to 500 mg/ml, wherein said formulation has a pH from 2.0 to 10.0. The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient adds solvents and/or diluents prior to use.

In another embodiment, the pharmaceutical formulation is a dried formulation (e.g. freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In a another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5.

In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment.

In a further embodiment, the formulation further comprises a pharmaceutically acceptable preservative. In a further embodiment, the formulation further comprises an isotonic agent. In a further embodiment, the formulation also comprises a chelating agent. In a further embodiment the formulation further comprises a stabilizer. In a further embodiment, the formulation further comprises a surfactant. For convenience reference is made to Remington: The Science and Practice of Pharmacy, 19th edition, 1995.

It is possible that other ingredients may be present in the peptide pharmaceutical formulation. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation.

Pharmaceutical compositions containing an antibody may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen. Administration of pharmaceutical compositions may be through several routes of administration, for example, subcutaneous, intramuscular, intraperitoneal, intravenous, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Suitable antibody formulations can also be determined by examining experiences with other already developed therapeutic monoclonal antibodies. Several monoclonal antibodies have been shown to be efficient in clinical situations, such as Rituxan (Rituximab), Herceptin (Trastuzumab) Xolair (Omalizumab), Bexxar (Tositumomab), Campath (Alemtuzumab), Zevalin, Oncolym and similar formulations may be used with the antibodies. For example, a monoclonal antibody can be supplied at a concentration of 10 mg/mL in either 100 mg (10 mL) or 500 mg (50 mL) single-use vials, formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. In another embodiment, the antibody is supplied in a formulation comprising about 20 mM Na-Citrate, about 150 mM NaCl, at pH of about 6.0.

### Diagnosis and Treatment of Disease

Methods of treating an individual, notably a human patient, using an anti-CD39 antibody as described herein are also provided for. In one embodiment, the disclosure provides for the use of an antibody as described herein in the preparation of a pharmaceutical composition for administration to a human patient. Typically, the patient suffers from, or is at risk for, cancer or an infectious disease (e.g. a viral infection, bacterial infection).

For example, in one aspect, provided is a method of restoring or potentiating the activity of lymphocytes in a patient in need thereof, comprising the step of administering a neutralizing anti-CD39 antibody to said patient. The antibody can be for example a human or humanized anti-CD39 antibody that specifically binds “vascular” CD39 without binding CD39-L1, L2, L3 and/or L4, which antibody reduces or abrogates the ATPase activity of human CD39 and which is not substantially bound by human CD16 (and optionally further is not bound by other human Fcγ receptors such as CD32a, CD32b or CD64). Such antibodies will have reduced unwanted side effects or toxicity due to lack of binding at isoforms other than vascular CD39, and will have reduced unwanted side effects or toxicity resulting from depletion or other Fc-mediated effects on CD39-expressing endothelial cells in the vasculature.

In one embodiment, the method is directed at increasing the activity of lymphocytes (e.g. T cells) in patients having a disease in which increased lymphocyte activity is beneficial or which is caused or characterized by immunosuppression, immunosuppressive cells, or, e.g., adenosine generated by CD4 T cells, CD8 T cells, B cells). The methods will be particularly useful for example patients having a solid tumor in which it is suspected the tumor microenvironment (and CD39-mediated adenosine production therein) may contribute to lack of recognition by the immune system (immune escape). The tumor may, for example, be characterized by CD39-expressing immune cells, e.g., CD4 T cells, CD8 T cells, B cells.

More specifically, the methods and compositions are utilized for the treatment of a variety of cancers and other proliferative diseases, and infectious diseases. Because these methods operate by reducing adenosine that inhibits the anti-target cell (e.g. anti-tumor) activity of lymphocytes and possibly additionally by increasing ATP that can increase the anti-tumor activity of lymphocytes, they are applicable to a very broad range of cancers and infectious disease. In one embodiment, the anti-CD39 compositions are useful to treat cancer in individuals who are poor responders to (or not sensitive to) treatment with agent that neutralizes the inhibitory activity of human PD-1, e.g. that inhibits the interaction between PD-1 and PD-L1. Representative examples of cancers that can be treated include in particular solid tumors in which adenosine in the tumor microenvironment may play a strong role in suppressing the anti-tumor immune response. In one embodiment, a human patient treated with an anti-CD39 antibody has liver cancer, bone cancer, pancreatic cancer, skin cancer, cancer of the head or neck, breast cancer, lung cancer, non-small cell lung cancer (NSCLC), castrate resistant prostate cancer (CRPC), melanoma, uterine cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers. The present disclosure is also applicable to treatment of metastatic cancers. Patients can be tested or selected for one or more of the above described clinical attributes prior to, during or after treatment.

In one embodiment the anti-CD39 antibody is used in the treatment of a cancer characterized by malignant cells expressing vascular CD39. In one embodiment, the anti-CD39 antibody is used in the treatment of a cancer characterized by malignant cells expressing vascular CD39 wherein the CD39-positive malignant cells do not substantially express CD39L1-, -L2, -L3 and/or -L4. In one embodiment, the anti-CD39 antibody is used in the treatment of a cancer in a patient who comprises detectable a soluble CD39 isoform, optionally CD39-L2 and/or -L4.

In one embodiment, the anti-CD39 antibody is administered in an amount effective to achieve and/or maintain in an individual (e.g. for 1, 2, 3, 4 weeks, and/or until the subsequent administration of antigen binding compound) a blood concentration of at least the EC₅₀, optionally the EC₇₀, optionally substantially the EC₁₀₀, for neutralization of the enzymatic activity of CD39. In one embodiment, the active amount of anti-CD39 antibody is an amount effective to achieve the EC₅₀, optionally the EC₇₀, optionally substantially the EC₁₀₀, for neutralization of the enzymatic activity of CD39 in an extravascular tissue of an individual. In one embodiment, the active amount of anti-CD39 antibody is an amount effective to achieve (or maintain) in an individual the EC₅₀, optionally the EC₇₀, optionally substantially the EC₁₀₀, for inhibition of neutralize the enzymatic activity of CD39.

Optionally, in one embodiment, in contrast to some antibodies that are directed to the depletion of CD39-expressing tumor cells by ADCC (which, e.g., can provide full efficacy at concentrations equal or substantially lower than that which provides receptor saturation), the anti-CD39 antibody does not exhibit substantial Fcγ receptor-mediated activity and is administered in an amount effective to neutralize the enzymatic activity of, optionally further CD39, without substantially causing down-modulation of CD39 expression, for a desired period of time, e.g. 1 week, 2 weeks, a month, until the next successive administration of anti-CD39 antibody.

In one embodiment, the anti-CD39 antibody is administered in an amount effective to achieve and/or maintain (e.g. for 1, 2, 3, 4 weeks, and/or until the subsequent administration of anti-CD39 antibody) in an individual a blood concentration of at least the EC₅₀, optionally the EC₇₀, optionally substantially the EC₁₀₀, for inhibition of CD39-mediated catabolism of ATP to AMP (e.g., by assessing neutralization of ATPase activity in B cells, optionally Ramos lymphoma cells, by quantifying hydrolysis of ATP to AMP, see Example 6). In one embodiment, the amount of anti-CD39 antibody is an amount effective to achieve (or maintain), in an extravascular tissue of an individual, the EC₅₀, optionally the EC₇₀, optionally substantially the EC₁₀₀, for inhibition of CD39-mediated catabolism of ATP to AMP.

In one embodiment, provided is a method for treating or preventing cancer in an individual, the method comprising administering to an individual having disease an anti-CD39 antibody in an amount that achieves or maintains for a specified period of time a concentration in circulation, optionally in an extravascular tissue of interest (e.g. the tumor or tumor environment), that is higher than the concentration required for 50%, 70%, or full (e.g. 90%) receptor saturation CD39-expressing cells in circulation (for example as assessed in PBMC). Optionally the concentration achieved is at least 20%, 50% or 100% higher than the concentration required for the specified receptor saturation.

In one embodiment, provided is a method for treating or preventing cancer in an individual, the method comprising administering to the individual an anti-CD39 antibody in an amount that achieves or maintains for a specified period of time a concentration in circulation, optionally in an extravascular tissue of interest (e.g. the tumor or tumor environment), that is higher than the EC₅₀, optionally EC₇₀ or optionally EC₁₀₀, for binding to CD39-expressing cells (e.g., as assessed by titrating anti-CD39 antibody on CD39-expressing cells, for example Ramos cells as in Example 3). Optionally the concentration achieved is at least 20%, 50% or 100% higher than the EC₅₀, optionally EC₇₀ or optionally EC₁₀₀, for binding to CD39-expressing cells.

The EC₅₀, EC₇₀ or the EC₁₀₀ can be assessed for example in a cellular assay for neutralization of the enzymatic activity of CD39 as shown in the Examples herein, e.g., neutralization of ATPase activity in B cells by quantifying hydrolysis of ATP to AMP (or ATP to downstream adenosine), see Example 6. “EC₅₀” with respect to neutralization of the enzymatic activity of CD39, refers to the efficient concentration of anti-CD39 antibody which produces 50% of its maximum response or effect with respect to neutralization of the enzymatic activity. “EC₇₀” with respect to neutralization of the enzymatic activity of CD39, refers to the efficient concentration of anti-CD39 antibody which produces 70% of its maximum response or effect. “EC₁₀₀” with respect to neutralization of the enzymatic activity of CD39, refers to the efficient concentration of anti-CD39 antibody which produces its substantially maximum response or effect with respect to such neutralization of the enzymatic activity.

In some embodiments, particularly for the treatment of solid tumors, the concentration achieved is designed to lead to a concentration in tissues (outside of the vasculature, e.g. in the tumor or tumor environment) that corresponds to at least the EC₅₀ or EC₇₀ for neutralization of the enzymatic activity, optionally at about, or at least about, the EC₁₀₀.

In one embodiment, the amount of anti-CD39 antibody is between 1 and 20 mg/kg body weight. In one embodiment, the amount is administered to an individual weekly, every two weeks, monthly or every two months.

In one embodiment provided is a method of treating a human individual having a cancer, comprising administering to the individual an effective amount of an anti-CD39 antibody of the disclosure for at least one administration cycle (optionally at least 2, 3, 4 or more administration cycles), wherein the cycle is a period of eight weeks or less, wherein for each of the at least one cycles, one, two, three or four doses of the anti-CD39 antibody are administered at a dose of I-20 mg/kg body weight. In one embodiment, the anti-CD39 antibody is administered by intravenous infusion.

Suitable treatment protocols for treating a human include, for example, administering to the patient an amount as disclosed herein of an anti-CD39 antibody, wherein the method comprises at least one administration cycle in which at least one dose of the anti-CD39 antibody is administered. Optionally, at least 2, 3, 4, 5, 6, 7 or 8 doses of the anti-CD39 antibody are administered. In one embodiment, the administration cycle is between 2 weeks and 8 weeks.

In one embodiment, provided is a method for treating or preventing a disease (e.g. a cancer, a solid tumor, a hematological tumor) in an individual, the method comprising administering to an individual having disease (e.g. a cancer, a solid tumor, a hematological tumor) an anti-CD39 antibody that neutralizes the enzymatic activity of CD39 for at least one administration cycle, the administration cycle comprising at least a first and second (and optionally a 3^{rd}, 4^{th}, 5^{th}, 6^{th}, 7^{th} and/or 8^{th} or further) administration of the anti-CD39 antibody, wherein the anti-CD39 antibody is administered in an amount effective to achieve, or to maintain between two successive administrations, a blood (serum) concentration of anti-CD39 antibody of at least 0.1 μg/ml, optionally at least 0.2 μg/ml, optionally at least 1 μg/ml, or optionally at least 2 μg/ml (e.g. for treatment of a hematological tumor), or optionally at least about 1 μg/ml, 2 μg/ml, 10 μg/ml, or 20 μg/ml, e.g. between 1-100 μg/ml, 1-50 μg/ml, 1-20 μg/ml, or 1-10 μg/ml (e.g. for treatment of a solid tumor, for treatment of a hematological tumor). In one embodiment, a specified continuous blood concentration is maintained, wherein the blood concentration does not drop substantially below the specified blood concentration for the duration of the specified time period (e.g. between two administrations of antibody, number of weeks, 1 week, 2 weeks, 3 weeks, 4 weeks), i.e. although the blood concentration can vary during the specified time period, the specified blood concentration maintained represents a minimum or “trough” concentration. In one embodiment, a therapeutically active amount of an anti-CD39 antibody is an amount of such antibody capable of providing (at least) the E050 concentration, optionally the EC₇₀ concentration optionally the EC₁₀₀ concentration, in blood and/or in a tissue for neutralization of the enzymatic activity of CD39 for a period of at least about 1 week, about 2 weeks, or about one month, following administration of the antibody.

Prior to or during a course of treatment with an anti-CD39 antibody of the disclosure, presence or levels or CD39-expressing cells, adenosine, ATP, ADP and/or AMP levels can be assessed within and/or adjacent to a patient's tumor to assess whether the patient is suitable for treatment (e.g. to predict whether the patient is likely to respond to treatment). Increased presence or levels or CD39-expressing cells, levels of adenosine, ATP, ADP and/or AMP may indicate an individual is suitable for treatment with (e.g. likely to benefit from) an anti-CD39 antibody of the disclosure (including but not limited to an antibody that inhibits substrate-bound CD39).

Prior to or during a course of treatment with an anti-CD39 antibody of the disclosure, adenosine, ADP and/or AMP levels can also be assessed within and/or adjacent to a patient's tumor to assess whether the patient is benefiting from treatment with an anti-CD39 antibody. Decreased levels of adenosine, ATP, ADP and/or AMP compared following an administration (or dosing of antibody) compared to levels prior to treatment (or dosing of antibody) may indicate an individual is benefiting from treatment with an anti-CD39 antibody of the disclosure (including but not limited to an antibody that inhibits substrate-bound CD39). Optionally, if a patient is benefiting from treatment with the anti-CD39 antibody, methods can further comprise administering a further dose of the anti-CD39 antibody to the patient (e.g., continuing treatment).

In one embodiment, assessing adenosine, ADP and/or AMP levels within and/or adjacent to a patient's tumor the tissue sample comprises obtaining from the patient a biological sample of a human tissue selected from the group consisting of tissue from a cancer patient, e.g., cancer tissue, tissue proximal to or at the periphery of a cancer, cancer adjacent tissue, adjacent non-tumorous tissue or normal adjacent tissue, and detecting adenosine, ATP, ADP and/or AMP levels within the tissue. The levels from the patient can be comparing the level to a reference level, e.g. corresponding to a healthy individual.

In one embodiment, the disclosure provides a method for the treatment or prevention of a cancer in an individual in need thereof, the method comprising:

a) detecting CD39-expressing cells (or adenosine, ATP, ADP and/or AMP) in circulation or in the tumor environment, optionally within the tumor and/or within adjacent tissue, and

b) upon a determination that CD39-expressing cells (or adenosine, ATP, ADP and/or AMP) are comprised in circulation or the tumor environment, optionally at a level that is increased compared to a reference level (e.g. corresponding to a healthy individual or an individual not deriving substantial benefit from an anti-CD39 antibody), administering to the individual an anti-CD39 antibody. The CD39-expressing cells may comprise tumor cells or leukocytes, for example circulating or tumor infiltrating cells, for example CD4 T cells, CD8 T cells, TReg cells, B cells.

In one embodiment, the disclosure provides a method for the treatment or prevention of a cancer in an individual in need thereof, the method comprising:

a) detecting cells in circulation that express vascular CD39 (e.g. from a blood sample), and

b) upon a detection of cells in circulation that express vascular CD39, optionally at a level that is increased compared to a reference level (e.g. corresponding to a healthy individual or an individual not deriving substantial benefit from an anti-CD39 antibody), administering to the individual an anti-CD39 antibody. The CD39-expressing cells may comprise tumor cells or leukocytes, for example circulating CD4 T cells, CD8 T cells, TReg cells, B cells.

Optionally, the anti-CD39 antibody specifically binds vascular CD39, e.g. the antibody binds a polypeptide having the sequence of SEQ ID NO: 1 but not does bind a secreted CD39 isoform polypeptide, e.g., a CD39-L2 and/or -L4 polypeptide. Optionally, the anti-CD39 antibody specifically binds vascular CD39, e.g. the antibody binds a polypeptide having the sequence of SEQ ID NO: 1 but not does bind a membrane bound CD39 isoform, e.g. CD39-L1 and/or -L3 polypeptide.

In one embodiment, the disclosure provides a method for the treatment or prevention of a cancer in an individual in need thereof, the method comprising:

a) detecting cells that express vascular CD39 in the tumor environment, optionally within the tumor and/or within adjacent tissue, and

b) upon a detection of cells in the tumor environment that express vascular CD39, optionally at a level that is increased compared to a reference level (e.g. corresponding to a healthy individual or an individual not deriving substantial benefit from an anti-CD39 antibody), administering to the individual an anti-CD39 antibody. The CD39-expressing cells may comprise tumor cells or leukocytes, for example tumor infiltrating cells, for example CD4 T cells, CD8 T cells, TReg cells, B cells. Optionally, the anti-CD39 antibody specifically binds vascular CD39, e.g. the antibody binds a polypeptide having the sequence of SEQ ID NO: 1 but not does bind a secreted CD39 isoform polypeptide, e.g., a CD39-L2 and/or -L4 polypeptide. Optionally, the anti-CD39 antibody specifically binds vascular CD39, e.g. the antibody binds a polypeptide having the sequence of SEQ ID NO: 1 but not does bind a membrane bound CD39 isoform, e.g. CD39-L1 and/or -L3 polypeptide.

Optionally, in any of the methods, detecting CD39-expressing cells (or adenosine, ATP, ADP and/or AMP) within the tumor environment comprises obtaining from the individual a biological sample that comprises cancer tissue and/or tissue proximal to or at the periphery of a cancer (e.g., cancer adjacent tissue, adjacent non-tumorous tissue or normal adjacent tissue), and detecting levels of CD39-expressing cells (or adenosine, ATP, ADP and/or AMP). CD39-expressing cells may comprise, for example, tumor cells, CD4 T cells, CD8 T cells, TReg cells, B cells.

A patient having a cancer can be treated with the anti-CD39 antibody with our without a prior detection step to assess expression of CD39 on circulating cells or on cells in the tumor microenvironment (e.g. on tumor cells, CD4 T cells, CD8 T cells, TReg cells, B cells). Optionally, the treatment method can comprise a step of detecting a CD39 nucleic acid or polypeptide in a biological sample from blood or of a tumor from an individual (e.g., in cancer tissue, tissue proximal to or at the periphery of a cancer, cancer adjacent tissue, adjacent non-tumorous tissue or normal adjacent tissue). A determination that a biological sample comprises cells expressing CD39 (e.g. prominently expressing; expressing CD39 at a high level, high intensity of staining with an anti-CD39 antibody, compared to a reference) indicates that the patient has a cancer that may have a strong benefit from treatment with an agent that inhibits CD39. In one embodiment, the method comprises determining the level of expression of a CD39 nucleic acid or polypeptide in a biological sample and comparing the level to a reference level corresponding to a healthy individual. A determination that a biological sample comprises cells expressing CD39 nucleic acid or polypeptide at a level that is increased compared to the reference level indicates that the patient has a cancer that can be advantageously treated with an anti-CD39 antibody of the disclosure. Optionally, detecting a CD39 polypeptide in a biological sample comprises detecting CD39 polypeptide expressed on the surface of a malignant cell, a CD4 T cell, CD8 T cell, TReg cell, B cell. In one embodiment, a determination that a biological sample comprises cells that prominently expresses CD39 nucleic acid or polypeptide indicates that the patients has a cancer that can be advantageously treated with an anti-CD39 antibody of the disclosure. “Prominently expressed”, when referring to a CD39 polypeptide, means that the CD39 polypeptide is expressed in a substantial number of cells taken from a given patient. While the definition of the term “prominently expressed” is not bound by a precise percentage value, in some examples a receptor said to be “prominently expressed” will be present on at least 10%, 20% 30%, 40%, 50° %, 60%, 70%, 80%, or more of the tumor cells taken from a patient.

Determining whether an individual has a cancer characterized by cells that express a CD39 polypeptide can for example comprise obtaining a biological sample (e.g. by performing a biopsy) from the individual that comprises cells from the cancer environment (e.g. tumor or tumor adjacent tissue), bringing said cells into contact with an antibody that binds an CD39 polypeptide, and detecting whether the cells express CD39 on their surface. Optionally, determining whether an individual has cells that express CD39 comprises conducting an immunohistochemistry assay.

The antibody compositions may be used in as monotherapy or combined treatments with one or more other therapeutic agents, including agents normally utilized for the particular therapeutic purpose for which the antibody is being administered. The additional therapeutic agent will normally be administered in amounts and treatment regimens typically used for that agent in a monotherapy for the particular disease or condition being treated. Such therapeutic agents include, but are not limited to anti-cancer agents and chemotherapeutic agents.

In one embodiment, the anti-CD39 neutralizing antibodies lack binding to human CD16 yet potentiate the activity of CD16-expressing effector cells (e.g. NK or effector T cells). Accordingly, in one embodiment, the second or additional second therapeutic agent is an antibody or other Fc domain-containing protein capable of inducing ADCC toward a cell to which it is bound, e.g. via CD16 expressed by an NK cell. Typically, such antibody or other protein will comprise a domain that binds to an antigen of interest, e.g. an antigen present on a tumor cell (tumor antigen), and an Fc domain or portion thereof, and will exhibit binding to the antigen via the antigen binding domain and to Fcγ receptors (e.g. CD16) via the Fc domain. In one embodiment, its ADCC activity will be mediated at least in part by CD16. In one embodiment, the additional therapeutic agent is an antibody having a native or modified human Fc domain, for example a Fc domain from a human IgG1 or IgG3 antibody. The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils. The term “ADCC-inducing antibody” refers to an antibody that demonstrates ADCC as measured by assay(s) known to those of skill in the art. Such activity is typically characterized by the binding of the Fc region with various FcRs. Without being limited by any particular mechanism, those of skill in the art will recognize that the ability of an antibody to demonstrate ADCC can be, for example, by virtue of it subclass (such as IgG1 or IgG3), by mutations introduced into the Fc region, or by virtue of modifications to the carbohydrate patterns in the Fc region of the antibody. Examples of antibodies that induce ADCC include rituximab (for the treatment of lymphomas, CLL, trastuzumab (for the treatment of breast cancer), alemtuzumab (for the treatment of chronic lymphocytic leukemia) and cetuximab (for the treatment of colorectal cancer, head and neck squamous cell carcinoma). Examples of ADCC-enhanced antibodies include but are not limited to: GA-101 (hypofucosylated anti-CD20), margetuximab (Fc enhanced anti-HER2), mepolizumab, MEDI-551 (Fc engineered anti-CD19), obinutuzumab (glyco-engineered/hypofucosuylated anti-CD20), ocaratuzumab (Fc engineered anti-CD20), XmAb® 5574/MOR208 (Fc engineered anti-CD19).

In one embodiment, the anti-CD39 neutralizing antibodies augments the efficacy of agents that neutralizes the inhibitory activity of human PD-1, e.g. that inhibits the interaction between PD-1 and PD-L1, notably in individuals who are poor responders to (or not sensitive to) treatment with agent that neutralizes the inhibitory activity of human PD-1. Accordingly, in one embodiment, the second or additional second therapeutic agent is an antibody or other agent that neutralizes the inhibitory activity of human PD-1.

Programmed Death 1 (PD-1) (also referred to as “Programmed Cell Death 1”) is an inhibitory member of the CD28 family of receptors. The complete human PD-1 sequence can be found under GenBank Accession No. U64863. Inhibition or neutralization the inhibitory activity of PD-1 can involve use of a polypeptide agent (e.g., an antibody, a polypeptide fused to an Fc domain, an immunoadhesin, etc.) that prevents PD-L1-induced PD-1 signalling. There are currently at least six agents blocking the PD-1/PD-L1 pathway that are marketed or in clinical evaluation. One agent is BMS-936558 (Nivolumab/ONO-4538, Bristol-Myers Squibb; formerly MDX-1106). Nivolumab, (Trade name Opdivo®) is an FDA-approved fully human IgG4 anti-PD-L1 mAb that inhibits the binding of the PD-L1 ligand to both PD-1 and CD80 and is described as antibody 5C4 in WO 2006/121168, the disclosure of which is incorporated herein by reference. For melanoma patients, the most significant OR was observed at a dose of 3 mg/kg, while for other cancer types it was at 10 mg/kg. Nivolumab is generally dosed at 10 mg/kg every 3 weeks until cancer progression. The terms “reduces the inhibitory activity of human PD-1”, “neutralizes PD-1” or “neutralizes the inhibitory activity of human PD-1” refers to a process in which PD-1 is inhibited in its signal transduction capacity resulting from the interaction of PD-1 with one or more of its binding partners, such as PD-L1 or PD-L2. An agent that neutralizes the inhibitory activity of PD-1 decreases, blocks, inhibits, abrogates or interferes with signal transduction resulting from the interaction of PD-1 with one or more of its binding partners, such as PD-L1, PD-L2. Such an agent can thereby reduce the negative co-stimulatory signal mediated by or through cell surface proteins expressed on T lymphocytes, so as to enhance T-cell effector functions such as proliferation, cytokine production and/or cytotoxicity.

MK-3475 (human IgG4 anti-PD1 mAb from Merck), also referred to as lambrolizumab or pembrolizumab (Trade name Keytruda®) has been approved by the FDA for the treatment of melanoma and is being tested in other cancers. Pembrolizumab was tested at 2 mg/kg or 10 mg/kg every 2 or 3 weeks until disease progression. MK-3475, also known as Merck 3745 or SCH-900475, is also described in WO2009/114335.

MPDL3280A/RG7446 (anti-PD-L1 from Roche/Genentech) is a human anti-PD-L1 mAb that contains an engineered Fc domain designed to optimize efficacy and safety by minimizing FcγR binding and consequential antibody-dependent cellular cytotoxicity (ADCC). Doses of 1, 10, 15, and 25 mg/kg MPDL3280A were administered every 3 weeks for up to 1 year. In phase 3 trial, MPDL3280A is administered at 1200 mg by intravenous infusion every three weeks in NSCLC.

AMP-224 (Amplimmune and GSK) is an immunoadhesin comprising a PD-L2 extracellular domain fused to an Fc domain. Other examples of agents that neutralize PD-1 may include an antibody that binds PD-L2 (an anti-PD-L2 antibody) and blocks the interaction between PD-1 and PD-L2.

Pidlizumab (CT-011; CureTech) (humanized IgG1 anti-PD1 mAb from CureTech/Teva), Pidlizumab (CT-011; CureTech) (see e.g., WO2009/101611) is another example; the agent was tested in thirty patients with rituximab-sensitive relapsed FL were treated with 3 mg/kg intravenous CT-011 every 4 weeks for 4 infusions in combination with rituximab dosed at 375 mg/m2 weekly for 4 weeks, starting 2 weeks after the first infusion of CT-011.

Further known PD-1 antibodies and other PD-1 inhibitors include AMP-224 (a B7-DC/IgG1 fusion protein licensed to GSK), AMP-514 described in WO 2012/145493, antibody MEDI-4736 (an anti-PD-L1 developed by AstraZeneca/Medimmune) described in WO2011/066389 and US2013/034559, antibody YW243.55.S70 (an anti-PD-L1) described in WO2010/077634, MDX-1105, also known as BMS-936559, is an anti-PD-L1 antibody developed by Bristol-Myers Squibb described in WO2007/005874, and antibodies and inhibitors described in WO2006/121168, WO2009/014708, WO2009/114335 and WO2013/019906, the disclosures of which are hereby incorporated by reference. Further examples of anti-PD1 antibodies are disclosed in WO2015/085847 (Shanghai Hengrui Pharmaceutical Co. Ltd.), for example antibodies having light chain variable domain CDR1, 2 and 3 of SEQ ID NO: 6, SEQ ID NO: 7 and/or SEQ ID NO: 8, respectively, and antibody heavy chain variable domain CDR1, 2 and 3 of SEQ ID NO: 3, SEQ ID NO: 4 or SEQ ID NO: 5, respectively, wherein the SEQ ID NO references are the numbering according to WO2015/085847, the disclosure of which is incorporated herein by reference. Antibodies that compete with any of these antibodies for binding to PD-1 or PD-L1 also can be used. An exemplary anti-PD-1 antibody is pembrolizumab (commercialized by Merck & Co. as Keytruda™, see, also WO 2009/114335 the disclosure of which is incorporated herein by reference).

In some embodiments, the PD-1 neutralizing agent is an anti-PD-L1 mAb that inhibits the binding of PD-L1 to PD-1. In some embodiments, the PD-1 neutralizing agent is an anti-PD1 mAb that inhibits the binding of PD-1 to PD-L1. In some embodiments, the PD-1 neutralizing agent is an immunoadhesin (e.g., an immunoadhesin comprising an extracellular or PD-1 binding portion of PD-L1 or PD-L2 fused to a constant region (e.g., an Fc region of an immunoglobulin sequence).

In the treatment methods, the CD39-binding compound and the second therapeutic agent can be administered separately, together or sequentially, or in a cocktail. In some embodiments, the antigen-binding compound is administered prior to the administration of the second therapeutic agent. For example, the CD39-binding compound can be administered approximately 0 to 30 days prior to the administration of the second therapeutic agent. In some embodiments, an CD39-binding compound is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the second therapeutic agent. In some embodiments, a CD39-binding compound is administered concurrently with the administration of the therapeutic agents. In some embodiments, a CD39-binding compound is administered after the administration of the second therapeutic agent. For example, a CD39-binding compound can be administered approximately 0 to 30 days after the administration of the second therapeutic agent. In some embodiments, a CD39-binding compound is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the second therapeutic agent.

### EXAMPLES

### Example 1: Generation of New Anti-huCD39 Antibodies

Cloning, Production and Purification of huCD39

Molecular Biology

The huCD39 protein was cloned from human PBMC cDNA using the following primers TACGACTCACAAGCTTGCCGCCACCATGGAAGATACAAAGGAGTC (SEQ ID NO: 60) (Forward) and CCGCCCCGACTCTAGATCACTTGTCATCGTCATCTTTGTAATCGACATAGGTGGAGTGG GAGAG (SEQ ID NO: 61) (Reverse). The purified PCR product was then cloned into an expression vector using the InFusion cloning system. A M2 tag was added in the C-terminal part of the protein for the purification step.

Expression and Purification of the huCD39 Proteins

After validation of the sequence cloned, CHO cells were nucleofected and the producing pool was then sub-cloned to obtain a cell clone producing the huCD39 protein. Supernatant from the huCD39 clone grown in roller was harvested and purified using M2 chromatography column and eluted using the M2 peptide. The purified proteins were then loaded onto a S200 size exclusion chromatography column. The purified protein corresponding to a monomer was formulated in a TBS PH7.5 buffer.

Immunization and Screen

To obtain anti-human CD39 antibodies, Balb/c mice were immunized with a recombinant human CD39-M2 extracellular domain recombinant protein. Mice received one primo-immunization with an emulsion of 50 μg CD39 protein and Complete Freund Adjuvant, intraperitoneally, a 2nd immunization with an emulsion of 50 μg CD39 protein and Incomplete Freund Adjuvant, intraperitoneally, and finally a boost with 10 μg CD39 protein, intravenously. Immune spleen cells were fused 3 days after the boost with X63.Ag8.653 immortalized B cells, and cultured in the presence of irradiated spleen cells. Hydridomas were plated in semi-solid methylcellulose-containing medium and growing clones were picked using a clonepix 2 apparatus (Molecular Devices).

Primary screen: Supernatant (SN) of growing clones were tested in a primary screen by flow cytometry using CHO cells expressing huCD39. Cells were stained with 0.1 μM and 0.005 μM Cell Trace Red, respectively. For the flow cytometry screening, all cells were equally mixed and the presence of reacting antibodies in supernanants was revealed by Goat anti-mouse polyclonal antibody (pAb) labeled with PE. Antibodies that bind huCD39 were cloned and produced as recombinant chimeric human IgG1 antibodies with a heavy chain N297Q (Kabat EU numbering) mutation which results in lack of N-linked glycosylation and lack of binding to human Fcγ receptors CD16A, CD16B, CD32A, CD32B and CD64.

### Example 2: Production of Antibodies I-391 and I-392 as Mutated Human IgG1

Antibody I-391 having the VH and Vk variable regions shown in SEQ ID NOS 6 and 7, respectively was produced as an Fc silent recombinant chimeric human IgG1 antibodies with a heavy chain N297Q (Kabat EU numbering) mutation which results in lack of N-linked glycosylation and reduces binding to human Fcγ receptors CD16A, CD16B, CD32A, CD32B and low residual binding to CD64.

Briefly, the VH and Vk sequences of the I-391 antibody were cloned into expression vectors containing the huIgG1 constant domains (harbouring the N297Q mutation) and the huCk constant domain respectively. The two obtained vectors were co-transfected into the CHO cell line. The established pool of cell was used to produce the antibody in the CHO medium. The antibody was then purified using protein A. The amino acid sequences of the respective heavy and light chains of I-391 are shown below. Antibody I-392 was produced in the same way using the same huIgG1 constant domains harbouring the N297Q mutation.

I-391 heavy chain sequence:
(SEQ ID NO: 62)
QIQLVQSGPELKKPGETVKISCKASGYTFRNYGMNWVKQAPGKGLKWMGW
INTYTGEPTYADDFKGRFAFSLATSASTAYLQISNLKNEDTATYFCARKA
YYGSNYYFDYWGQGTTLTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
QSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
I-391 light chain sequence:
(SEQ ID NO: 63)
DIVMTQSHKFMSTSVGDRVSITCKASQDVSTAVAWYQQKPGQSPKLLIYS
ASYRYTGVPDRFTGSGSGTDFTFTISTVQAEDLAVYYCQQHYTTPPYTFG
GGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

### Example 3: Ab Titration on Rec CD39 Protein by Flow Cytometry

Antibody I-391 was tested for binding to soluble recombinant human and cynomolgus CD39. Briefly, 1×10⁵ HEK-huCD39or cynoCD39 cells were incubated with various concentration of unlabeled anti-CD39 antibody or isotype control (IC) from 99 nM to 0.045 nM, for 30 minutes at 4° C. After washes, cells were incubated with Goat anti-mouse H+L labeled secondary antibody for 30 min at 4° C.

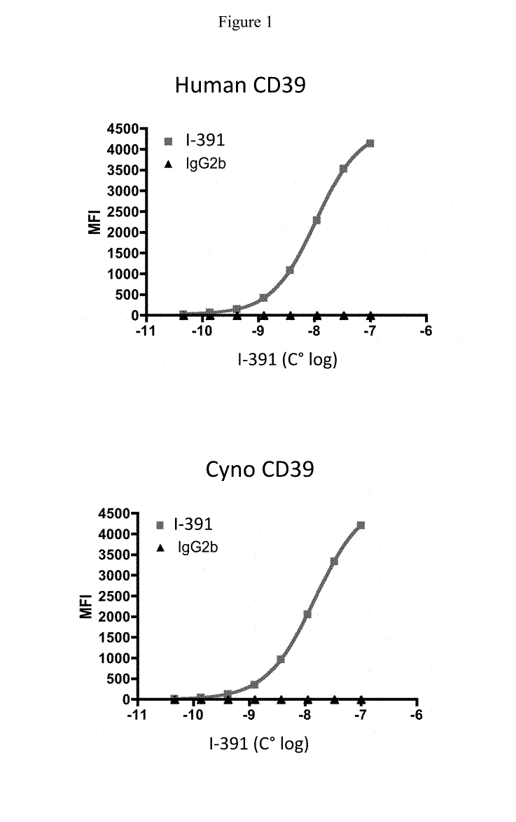

Results are shown in FIG. 1. Antibody I-391 bound both human and cynomolgus vascular CD39. EC₅₀ values for binding to human CD39 was 14.9 nM while binding to cynomolgus CD39 was 10.6 nM.

### Example 4: ELISA Titration on CD39-L1, L2, L3, L4 Isoforms

Antibody I-391 and I-392 were tested for binding to recombinant human CD39 isoforms (Rec-huCD39 isoforms) having amino acid sequences shown below were coated in 96-well plate in PBS 1× at 500 ng/ml or 1 μg/ml at 4° C. overnight. Wells were washed in TBS Tween 20, and further saturated 2H at RT in TBS Blocking buffer. Dose range concentration of primary antibody was incubated in TBS blocking buffer for 2h at RT. Wells were washed in TBS Tween 20. Secondary Antibody (GAM-HRP or GAH-HRP in TBS blocking buffer) was incubated for 1H at RT, and was revealed with TMB. Optical density was measured on Enspire at OD=450.

Amino Acid Sequence of the Cloned huCD39 (Vascular Isoform):
Human CD39-L1, also known as NTPDase2 or ENTPD2:

| (SEQ ID NO: 2) | |
|---|---|
| 1 | magkvrsllp plllaaagla gllllcvptr dvreppalky givldagssh tsmfiykwpa |
| 61 | dkendtgivg qhsscdvpgg gissyadnps gasqslvgcl eqalqdvpke rhagtplylg |
| 121 | atagmrllnl tnpeastsvl mavthtltqy pfdfrgaril sgqeegvfgw vtanyllenf |
| 181 | ikygwvgrwf rprkgtlgam dlggastqit fettspaedr asevqlhlyg qhyrvythsf |
| 241 | lcygrdqvlq rllasalqth gfhpcwprgf stqvllgdvy qspctmaqrp qnfnssarvs |
| 301 | lsgssdphlc rdlvsglfsf sscpfsrcsf ngvfqppvag nfvafsaffy tvdflrtsmg |
| 361 | lpvatlqqle aaavnvcnqt waqqllsrgy gfderafggv ifqkkaadta vgwalgymln |
| 421 | ltnlipadpp glrkgtdfss wvvllllfas allaalvlll rqvhsaklps ti |

Human CD39-L2, also known as NTPDase6 or ENTPD6:

| (SEQ ID NO: 3) | |
|---|---|
| 1 | mkkgiryets rktsyifqqp qhgpwqtrmr kisnhgslrv akvayplglc vgvfiyvayi |
| 61 | kwhratatqa ffsitraapg arwgqqahsp lgtaadghev fygimfdags tgtrvhvfqf |
| 121 | trppretptl thetfkalkp glsayaddve ksaqgirell dvakqdipfd fwkatplvlk |
| 181 | ataglrllpg ekaqkllqkv kevfkaspfl vgddcvsimn gtdegvsawi tinfltgslk |
| 241 | tpggssvgml dlgggstqia flprvegtlq asppgyltal rmfnrtykly sysylglglm |
| 301 | sarlailggv egqpakdgke lvspclspsf kgewehaevt yrvsgqkaaa slhelcaarv |
| 361 | sevlqnrvhr teevkhvdfy afsyyydlaa gvglidaekg gslvvgdfei aakyvcrtle |
| 421 | tqpqsspfsc mdltyvslll qefgfprskv lkltrkidnv etswalgaif hyidslnrqk |
| 481 | spas |

Human CD39-L3, also known as NTPDase3 or ENTPD3:

| (SEQ ID NO: 4) | |
|---|---|
| 1 | mftvltrqpc eqaglkalyr tptiialvvl lvsivvlvsi tviqihkqev lppglkygiv |
| 61 | ldagssrttv yvyqwpaeke nntgvvsqtf kcsvkgsgis sygnnpqdvp rafeecmqkv |
| 121 | kgqvpshlhg stpihlgata gmrllrlqne taanevlesi qsyfksqpfd frgaqiisgq |
| 181 | eegvygwita nylmgnflek nlwhmwvhph gvettgaldl ggastqisfv agekmdlnts |
| 241 | dimqvslygy vytlythsfq cygrneaekk flamllqnsp tknhltnpcy prdysisftm |
| 301 | ghvfdslctv dqrpesynpn dvitfegtgd pslckekvas ifdfkachdq etcsfdgvyq |
| 361 | pkikgpfvaf agfyytasal nlsgsfsldt fnsstwnfcs qnwsqlplll pkfdevyars |
| 421 | ycfsanyiyh lfvngykfte etwpqihfek evgnssiaws lgymlsltnq ipaesplirl |
| 481 | pieppvfvgt lafftaaall claflaylcs atrrkrhseh afdhavdsd |

Human CD39-L4, also known as NTPDase5 or ENTPDS:

| (SEQ ID NO: 5) | |
|---|---|
| 1 | matswgtvff mlvvscvcsa vshrnqqtwf egiflssmcp invsastlyg imfdagstgt |
| 61 | rihvytfvqk mpgqlpileg evfdsvkpgl safvdqpkqg aetvqgllev akdsiprshw |
| 121 | kktpvvlkat aglrllpehk akallfevke ifrkspflvp kgsvsimdgs degilawvtv |
| 181 | nfltgqlhgh rqetvgtldl ggastqitfl pqfektleqt prgyltsfem fnstyklyth |
| 241 | sylgfglkaa rlatlgalet egtdghtfrs aclprwleae wifggvkyqy ggnqegevgf |
| 301 | epcyaevlrv vrgklhqpee vqrgsfyafs yyydravdtd midyekggil kvedferkar |
| 361 | evcdnlenft sgspflcmdl syitallkdg fgfadstvlq ltkkvnniet gwalgatfhl |
| 421 | lqslgish |

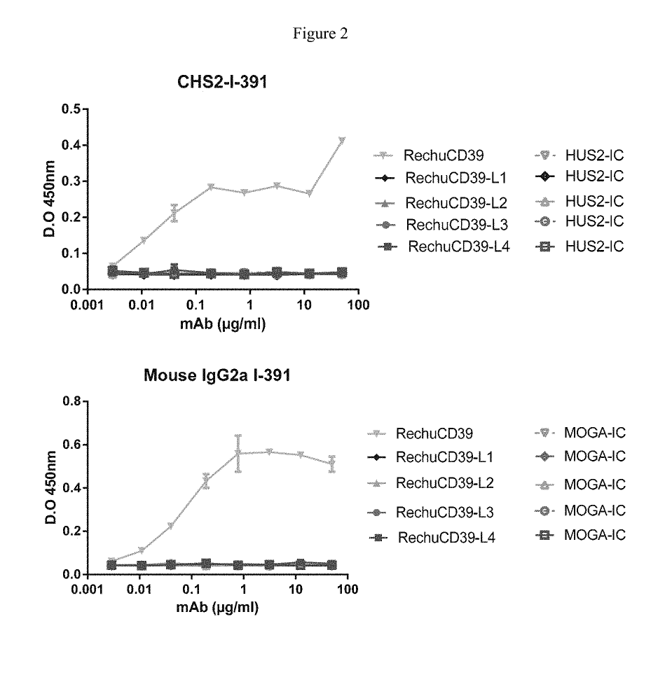

Neither antibody I-391 nor I-392 bound to the vascular CD39 but not to any of the CD39 isoforms CD39-L1, -L2, -L3 or -L4. Isotype control antibodies (IC) did not bind to any CD39 or CD39-L molecule. Results are shown in FIG. 2 for I-391. The top panel shows antibody I-391 or isotype control having a human IgG1 Fc domain with a N297Q mutation to lose binding to human Fcγ receptors; the bottom panel shows antibodies with Fc domain of mouse IgGa isotype (MOGA).

### Example 5: Neither I-391 Nor I-392 Induce or Increase CD39 Down-Modulation

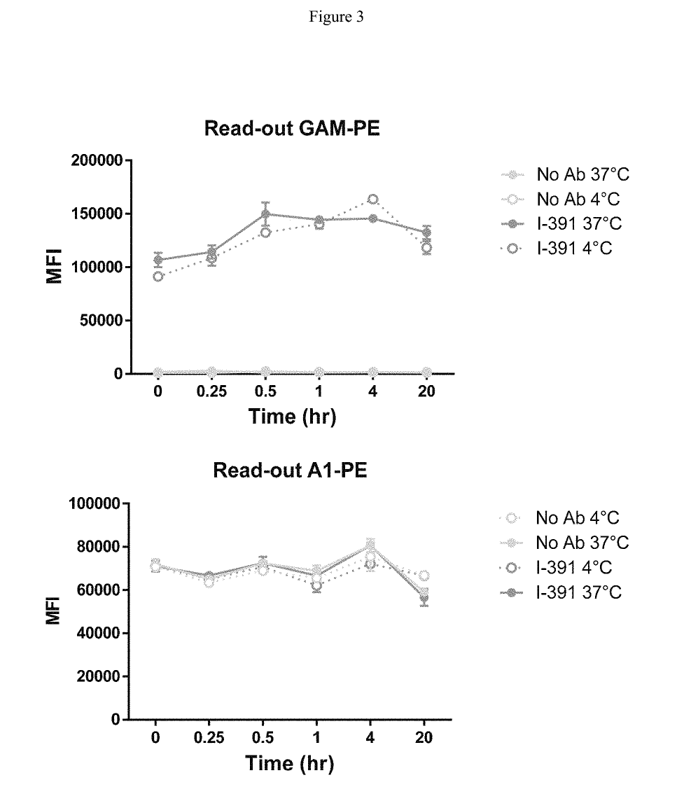

I-391 and I-392 were each incubated on Ramos human lymphoma cells at 10 μg/ml, during the indicated time period, at 4° C. or 37° C. Cells were then stained with either GAM-PE to reveal the presence of bound I-391 or I-392 Ab at cell surface, or with A1-PE, an anti-huCD39 Ab that does not compete with I-391 or I-392, to reveal the total amount of human CD39 at cell surface. As shown in FIG. 3 for I-391 (top panel: I-391; bottom panel: A1), following incubation with I-391 or I-392, CD39 expression remained stable and comparable to incubation in the absence of Ab, and no decrease in bound I-391 or I-392 could be detected, indicated that I-391 and I-392 do not induce CD39 down modulation nor CD39 internalization.

### Example 6: 1-391 and I-392 are Capable of Substantially Full Neutralization of ATPase Activity

The inhibition of CD39 enzymatic activity by antibodies was evaluated by Maldi TOF mass spectrometry (production of AMP). This assay represents an assay that is relatively insensitive to down-modulation of CD39 expression. Briefly, 10⁵ Ramos human lymphoma cells were incubated overnight at 37° C. with I-391 or I-392, or a chemical CD39 inhibitor (ARL 100 μM). After washes, cells were incubated with anti-CD39 antibodies, isotype control, or ARL and 50 μM ATP for 30 minutes at 4° C. in PBS. AMP generated is quantified in supernatants by Maldi TOP.

I-391 are I-392 were both very potent at blocking CD39 enzymatic activity. The calculated EC₅₀ (inhibition of 50% of the enzymatic activity of CD39 expressed by 100,000 Ramos cells) is 1.2 nM (n=3). The maximum inhibition achieved is 93.4%. Isotype control had no effect.

### Example 7: Comparative ATPase Blockade by Internalizing and Non-Internalizing mAb

CD39 blockade by I-391 was compared to other anti-CD39 antibodies and to the chemical inhibitor ARL. Comparator anti-CD39 bodies tested included antibody “A1” an antibody that induces at least partial down-modulation of cell surface CD39 and/or dissociates from CD39 rapidly, available from ABD Serotec, product code MCA1268GA; reported for example in Hausler et al. Am J Transl Res. 2014 Jan. 15; 6(2):129-39).

The inhibition of CD39 enzymatic activity by antibodies was evaluated by Maldi TOF mass spectrometry (production of AMP). Briefly, 10⁵ Ramos human lymphoma cells were incubated overnight at 37° C. with anti-CD39 antibodies or a chemical CD39 inhibitor (ARL 100 μM). After washes, cells were incubated with anti-CD39 antibodies, isotype control, or ARL and 50 μM ATP for 30 minutes at 4° C. in PBS. AMP generated is quantified in supernatants by Maldi TOP. Antibodies were used at 33 nM and ARL at 100 nM.

Antibody I-391 led to a strong inhibition of AMP generated and again was very potent at blocking CD39 enzymatic activity. The chemical CD39 inhibitor ARL led to a strong inhibition of AMP generated, but resulted in significantly less CD39 blockade than I-391. Antibody A1 results in minimal detectable reduction in AMP generated. In summary I-391 had vastly superior CD39 blocking ability compared to the antibodies A1 or compared to the chemical inhibitor (although non-specific).

### Example 8: Epitope Mapping

In order to define the epitopes of anti-CD39 antibodies, we designed CD39 mutants defined by substitutions of amino acids exposed at the molecular surface over the surface of CD39. Mutants were transfected in Hek-293T cells, as shown in the table below. The targeted amino acid mutations in the table 1 below are shown using numbering of SEQ ID NO: 1.

TABLE 1

| Mutant | Substitutions | | | | | | |
|---|---|---|---|---|---|---|---|
| 1 | V77G | H79Q | Q445K | G446D | | | — |
| 2A | V81S | E82A | R111A | V115A | | | |
| 2B | E110A | R113T | E114A | | | | |
| 3 | R118A | S119A | Q120K | Q122H | E123A | | |
| 4 | D150A | E153S | R154A | S157K | N158A | L278F | |
| 5 | Q96A | N99A | E143A | R147E | | | |
| 6 | K188R | Replacement of the residues 190 to 206 | | | | | |
| | | by KTPGGS | | | | | |
| 7 | A272S | N274A | I276S | R278A | | | |
| 8 | S293A | K297G | K302A | E305A | T307K | Q311A | |
| 9 | K287E | K288A | V289A | E314R | | | |
| 10A | Q353A | D355S | E436A | H437Q | | | |
| 10B | H429A | T431A | A432D | D433A | | | — |
| 11 | N370K | L371K | E374A | K375G | -377V | V378S | — |
| 12 | K389N | Q393K | P394S | E397A | | | — |
| 13 | A403P | G404A | K406A | E407A | | | — |
| 15 | K87A | E100A | D107A | | | | |
| 16 | Q322A | Q323A | Q326A | E330K | | | |
| 17 | N333A | S335A | Y336G | N345A | | | |
| 18 | Q227A | I229S | D233A | Q237A | | | |
| 19 | R138A | M139A | E142K | | | | |

### Generation of Mutants

CD39 mutants were generated by PCR. The sequences amplified were run on agarose gel and purified using the Macherey Nagel PCR Clean-Up Gel Extraction kit (reference 740609). The purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO2 incubator for 48 hours prior to testing for transgene expression.

### Flow Cytometry Analysis of Anti-CD39 Binding to the HEK293T Transfected Cells

Dose-ranges of I-391 and A1 antibodies (10-2.5-0.625-0.1563-0.0391-0.0098-0.0024-0.0006 μg/ml) were tested on the 20 generated mutants by flow cytometry. I-391 antibody lost binding to mutant 5 of CD39, but not to any other mutant. Mutant 5 contains amino acid substitutions at residues Q96, N99, E143 and R147 indicating that one or more, or all of, the residues of the mutant are important to the core epitope of this antibody. Antibody A1 lost binding to mutants 7, 16 and 17. Mutant 7 contains amino acid substitutions at residues A272, N274, 1276 and R278, indicating that these residues are important to the core epitope of A1; Mutant 16 contains amino acid substitutions at residues, Q332, Q323, Q326 and E330, indicating that these residues are also important to the core epitope of A1. Mutant 17 contains amino acid substitutions at residues N333, S335, Y336 and N345, indicating that these residues are also important to the core epitope of A1.

### Example 8: Study of Anti-CD39/CD39 Complexes by X-Ray Diffraction

### Purification and Cystallogenesis

Protein production: Anti-CD39 antibody having the VH and VL CDRs of I-392 (the parental VH and VL of SEQ ID NOS: 8 and 9) was modified by introduction of human VH and VL acceptor frameworks. This antibody, produced as a human IgG1 lacking binding to human Fc receptors and was found to retain CD39 binding and neutralization of ATPase activity with a potency comparable to parental I-391, without induction of intracellular internalization of CD39, as also observed for parental I-391 antibody. The antibody furthermore lost binding to CD39 mutants 5 (shown in Example 7) but not to any other mutant, and competes for binding to CD39 with antibody I-391. The VH and Vk sequences of each antibody were cloned into vectors containing the huIgG1 CH1 constant domain and the huCk constant domain respectively. The two obtained vectors were co-transfected into the CHO cell line. The established pool of cell was used to produce the Fab antibody in the CHO medium. CD39 protein was produced in CHO cells using standard methods.

Protein Purification:

Antibody Fab fragments were purified in two steps, by affinity chromatography on Nickel-beads (Ni-NTA) followed by Size Exclusion Chromatography (SEC).

CD39/Fab complexes were purified in five steps. First, purified Fab were added to CD39 recombinant protein culture supernatant in order to form the complexes directly in culture medium. Complexes were purified from culture supernatant by affinity chromatography on Nickel-beads thanks to the Fab his tag. Ni-NTA purified complexes were then separated from free Fabs by ion exchange chromatography (IEC). CD39/Fab complexes were treated with PNGaseF in order to reduce the complexity of the antigen glycosylations. Finally, deglycosylated complexes were separated from PNGaseF by a second SEC and concentrated to about 15 mg/mL for crystallogenesis. Crystallogenesis was performed separately on ab Fab alone and CD39/fab complexes by an automated process using standard crystallogneneis kit, Wizard, MDL and Morpheus. Anti-CD39 Fab were crystallized in 0.1M Mes pH 6.5, 1.8M ammonium sulfate buffer; Crystals were frozen in 30% glycerol cryoprotectant and analysed at the Soleil synchrotron in Saclay using the Proxima 1 beamline. Fab/CD39 complexes were crystallized in 0.1M citrate pH5.5, 2M ammonium sulfate buffer. Crystals were frozen in 20% glycerol cryoprotectant and analysed at the IBS synchrotron in Grenoble.

Crystals of Fab and of Ag/Ab complexes diffracted at 2.14 and 2.26 Å respectively. Structures were solved by molecular isomorphous replacement (MIR) using pdb templates of Fabs and pdb templates of the rat CD39 molecule.

### Results

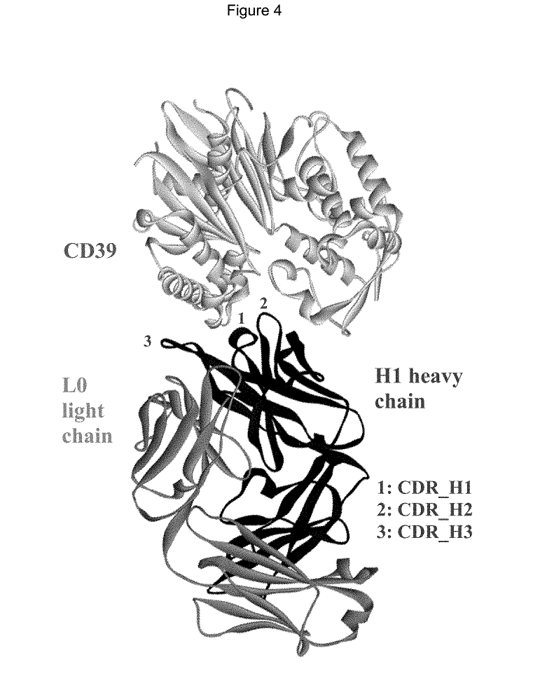

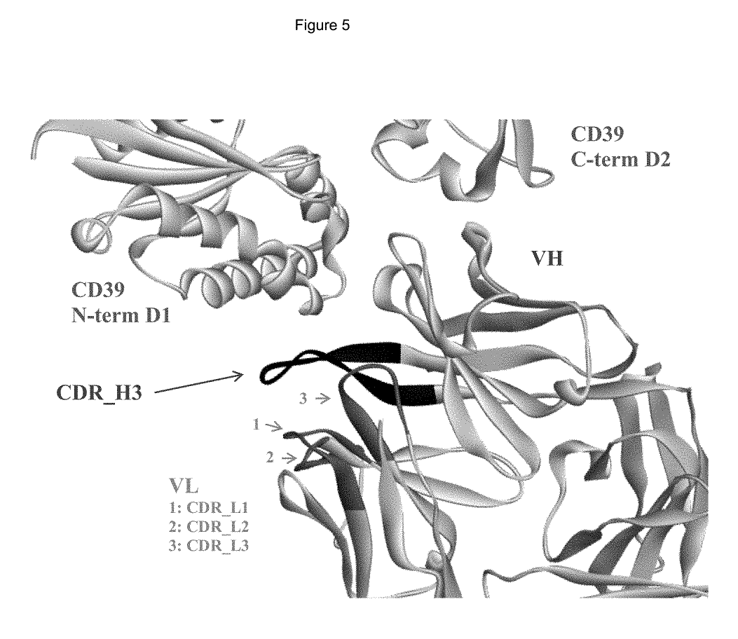

The 3-dimensional structure showed that binding of the neutralizing anti-CD39 to the target antigen CD39 entirely relies on the heavy chain variable domain (Summary table 2; Table 3; FIG. 4). The anti-CD39 antibody light chain does not contact the antigen directly (FIG. 4 and FIG. 5).

A total of 37 heavy chain residues are interfacing with CD39. Of these, 11 (˜30%) are Kabat framework (FR) residues and 26 (˜70%) are Kabat complementarity determining region (CDR) residues. 7 of the interfacing FR residues contact the glycan at the asparagine residue at position 292 of CD39 and 4 are interfacing with the CD39 protein but all have a minor contribution to the interface. Among the 11 FR residues facing the antigen, three were not conserved from the parental murine antibody (see Table 4). The parental residues were substituted by human residues showing very conservative physicochemical properties and structures: A68 replaced by V, E72 replaced by D and A72a replaced by T. Substitution of FR parental interfacing residues with human ones had no impact on antigen binding or antibody blocking activity.

Although the anti-CD39 light chain does not contact CD39 directly it appears to play an important role in the spatial organization of the heavy chain paratope. Indeed, the anti-CD39 heavy chain has a long CDR_H3 which strongly interacts with the light chain CDRs (FIG. 5; Table 5). The CDR_H3 is positioned just above the light chain CDRs, between the antigen and the V_{L} domain. Light chain CDRs form numerous interactions with CDR_H3. The light chain CDRs drive the orientation and the positioning of the CDR_H3 loop, they restrain the flexibility and motion of the CDR_H3 loop and they contribute indirectly to the binding to CD39. Interestingly, the CDR_H3 has a particularly high number of aromatic residues (principally tyrosines), and these aromatic residues permit a light chain/CDR_H3/CD39 matrix where the CDR_H3 is trapped between the V_{L} CDRs that, together with some FR residues, form a paratope directed to the CDR_H3, and CD39. The matrix is stabilized by several pi-interactions between aromatic residues in CDR_H3 and respective contact residues in the V_{L} CDRs and CD39.

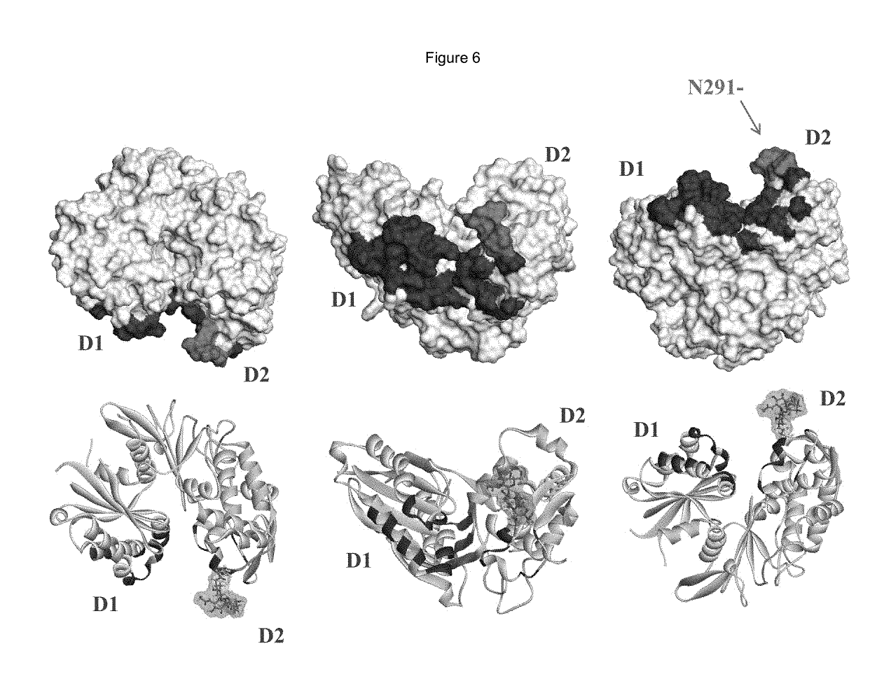

The anti-CD39 heavy chain binds to both the CD39 N-terminal domain 1 and C-terminal domain 2 of CD39). The anti-CD39 binding site is located at the apex of the two CD39 domains and at the entry of the catalytic cleft (FIG. 6; Table 2; Table 6). The N-terminal domain 1 of CD39 has a major contribution to the epitope (Table 6), and half (13) of the 26 heavy chain paratope residues form a direct bond with CD39-N-terminal domain 1. In contrast, only two heavy chain paratope residues form a direct bond with the C-terminal domain 2 of CD39. Instead, the C-terminal domain 2 of CD39 interacts with anti-CD39 antibody by the N292-sugar moiety and 8 AA residues located at the domain apex and cleft entry (Table 7). The 8 amino acid residues include both CDR (in Kabat CDR2) and framework residues (in both Kabat FR1 and FR3).

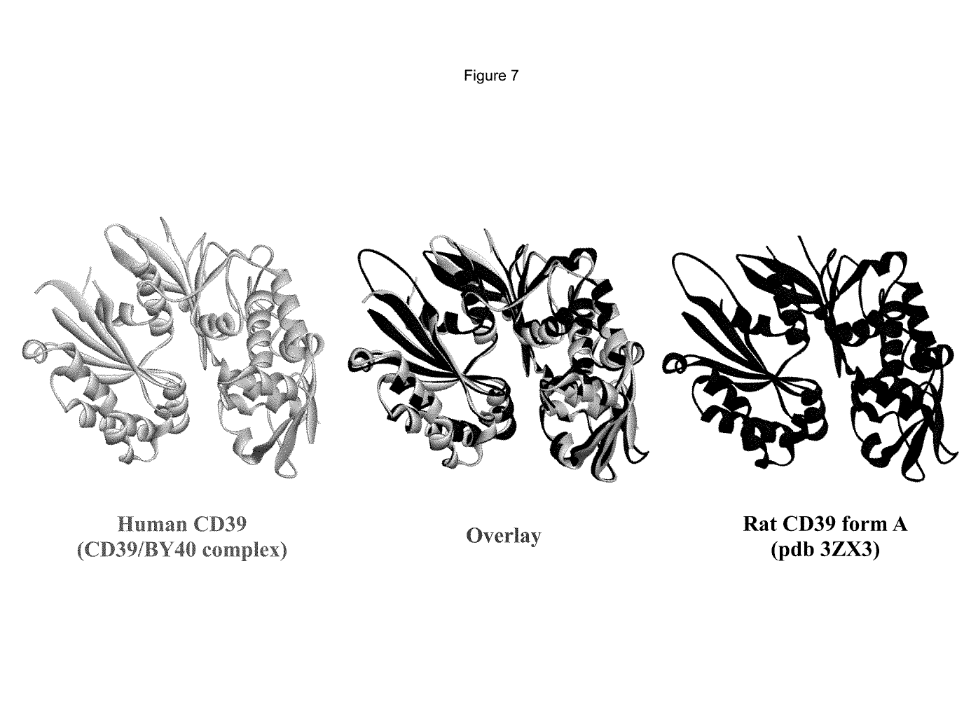

The fact that anti-CD39 antibody binds simultaneously to both CD39 domains 1 and 2, the latter optimized by binding via the N292-sugar moiety, likely explains how the antibody blocks the enzymatic activity. Indeed, based on structural data available in the literature (PDB database, reference 3ZX3) domain motion may be a key parameter required for the ability of CD39 to hydrolyse substrate. Moreover, the human CD39 structure obtained from the anti-CD39/CD39 co-crystal complex shows only one unique enzyme conformation corresponding to one fixed relative positioning of the two domains. On the contrary, rat CD39 when crystalized alone (pdb entry 3ZX3) exists under different conformations (i.e. with slightly different positions of the domains 1 and 2). The human CD39/anti-CD39 frozen conformation perfectly superimposes with rat CD39 form A of the pdb crystal 3ZX3 (FIG. 7). Binding of the antibody to both domains at the same time thus likely inhibits domain motion and block the enzyme in a given frozen status.

TABLE 2
VH residues interfacing with CD39

| Kabat | Kabat | | | | Location of contact | |
|---|---|---|---|---|---|---|
| position in | position in | | | | residue in CD39 (N- | PISA |
| VH; facing | VH; facing | | | Contact | terminal domain is | confirmation |
| CD39 | CD39 N292- | Location in | | residue in | Domain 1; C-terminal | of h-bond and |
| protein | linked glycan | VH | Bond | CD39 | domain is Domain 2) | salt bridge |
| | K19 | FR | NA | NAG31 | Domain 2 | |
| T30 | | FR | h-bond | CD39-Q96 | Domain 1 | YES |
| H31 | | CDR H1 | Pi-alkyl | CD39-L144 | Domain 1 | |
| | | | h-bond/salt bridge | CD39-E140 | | YES |
| Y32 | | CDR H1 | NA | | | |
| G33 | | CDR H1 | h-bond | CD39-Q96 | Domain 1 | |
| W50 | | CDR H2 | Hydrophobic | CD39-K97 | Domain 1 | |
| N52 | | CDR H2 | h-bond | CD39-V95 | Domain 1 | YES |
| T52a | | CDR H2 | h-bond | CD39-Q96 | Domain 1 | YES |
| Y53 | | CDR H2 | h-bond | CD39-L137 | Domain 1 | |
| | | | Pi-alkyl | CD39-L137 | | |
| | | | Pi-alkyl | CD39-V95 | | |
| T54 | | CDR H2 | h-bond | CD39-K298 | Domain 2 | |
| G55 | | CDR H2 | NA | NA | | |
| E56 | | CDR H2 | Salt bridge/h-bond | CD39-K97 | Domain 1 | YES |
| | P57 | CDR H2 | NA | NAG31 | Domain 2 | |
| T58 | | CDR H2 | NA | NA | | |
| | Y59 | CDR H2 | h-bond | NAG32 | Domain 2 | YES |
| | G65 | CDR H2 | NA | NAG32 | Domain 2 | |
| | F67 | FR3 | NA | NAG32 | Domain 2 | |
| | V68 | FR3 | NA | NAG31 | Domain 2 | |
| | | | | NAG32 | | |
| | | | | FUC33 | | |
| | F69 | FR3 | NA | NAG31 | Domain 2 | |
| | | | | NAG32 | | |
| | S70 | FR3 | NA | NAG31 | Domain 2 | |
| | | | h-bond | FUC33 | | |
| L71 | L71 | FR3 | NA | FUC33 | Domain 2 | |
| | | | h-bond | CD39-S294 | | |
| D72 | | FR3 | NA | NA | | |
| T72a | | FR3 | NA | NA | | |
| S72b | | FR3 | NA | NA | | |
| | Q78 | FR3 | NA | FUC33 | Domain 2 | |
| R95 | | CDR H3 | H bond | CD39-Q96 | Domain 1 | YES |
| R96 | | CDR H3 | NA | NA | | |
| E98 | | CDR H3 | NA | NA | | |
| G99 | | CDR H3 | H-bond | CD39-R154 | Domain 1 | YES |
| Y100a | | CDR H3 | NA | NA | | |
| V100b | | CDR H3 | Hydrophobic | CD39-V151 | Domain 1 | |
| F100c | | CDR H3 | NA | NA | | |
| Y100d | | CDR H3 | Amide Pi-stacked | CD39-Q96 | Domain 1 | |
| | | | h-bond | CD39-K97 | | YES |
| | | | h-bond | CD39-V98 | | YES |
| Y100e | | CDR H3 | Pi-Pi stacked | VL-Y49 | Domain 1 | |
| F100f | | CDR H3 | Pi-donor h-bond | VL-Q89 | Domain 1 | |
| D101 | | CDR H3 | NA | NA | | |
| Y102 | | CDR H3 | NA | NA | | |

TABLE 3
Buried surface at the antigen/antibody interface.

| Ab | Ag | Buried surface (Å⁻) |
|---|---|---|
| H1 | CD39 protein | 810.2 |
| H1 | N292-sugar | 46.9 |
| | | 93.3 |
| | | 95.1 |
| H1 | CD39 | 1045.5 |

TABLE 4
VH FR residues of humanized and parental antibody interfacing
with CD39

| Humanized HC:H1 | Parental mouse VH |
|---|---|
| K19 | K19 |
| T30 | T30 |
| F67 | F67 |
| V68 | A68 |
| F69 | F69 |
| S70 | S70 |
| L71 | L71 |
| D72 | E72 |
| T72a | A72a |
| S72b | S72b |
| Q78 | Q78 |

TABLE 5
VL residues interfacing with Heavy chain CDR H3

| Kabat | Light chain L0 | Location | Nature of bond | Target residue |
|---|---|---|---|---|
| S31 | S31 | CDR L1 | NA | NA |
| Y32 | Y32 | CDR L1 | Hydrogen bond | CDR H3-Y105 |
| F33 | F33 | CDR L1 | NA | NA |
| S34 | S34 | CDR L1 | NA | NA |
| Y49 | Y49 | FR2 | Pi-Stacking | CDR H3-Y109 |
| T50 | T50 | CDR L2 | NA | NA |
| Q89 | Q89 | CDR L3 | Pi-donor H bond | CDR H3-F110 |
| H91 | H91 | CDR L3 | Hydrogen bond | CDR H3-Y108 |

TABLE 6
CD39 interfacing residues

| Position | | | | |
|---|---|---|---|---|
| on CD39 | Location | Bond | Target residue | Comment |
| S92 | NTD1/groove | NA | NA | S92 and K93 are partially buried at the CDR H2/ |
| K93 | NTD1/groove | NA | NA | CD39 interface. Close to CDR H2-T55 |
| V95 | NTD1/groove | Pi-Alkyl | CDR H2-Y54 | V95 is 100% buried at the interface and has a direct |
| | entry | h-bond | CDR H2-N52 | contact with CDR H2-Y54 and N52 |
| Q96 | NTD1/groove | h-bonds | VH: T30, G33, | Q96 is a key residue of the Ag/Ab interface. |
| | entry | amide-pi | N52, R99 | It contacts many CDR residues including the three |
| | | stacked | Y108 | VH CDRs |
| K97 | NTD1/apex | h-bond | E57 | K97 is almost completely buried at the interface and |
| | | salt bridge | | it forms strong salt bridge and h-bond with |
| | | | | CDR H2-E57 |
| V98 | NTD1/apex | Potential | Y101 | V98 is 100% buried at the interface and it may form |
| | | Pi-alkyl | | a hydrophobic interaction with CDR H3-Y101 |
| N99 | NTD1/apex | NA | NA | N99 is partially buried at the interface. Close to the |
| | | | | CDR H3. |
| E100 | NTD1/apex | NA | NA | Minor contribution to the interface. |
| L136 | NTD1/groove | NA | NA | Exposed at the molecular surface. |
| | | | | Minor contribution to the interface. |
| L137 | NTD1/apex | Pi-alkyl | CDR H2-Y54 | L137 is located in the groove at the NTD1 apex and |
| | | | | oriented inside the domain hydrophobic cavity. |
| | | | | It likely forms a Pi-alkyl interaction with CDR H2- |
| | | | | Y54. It also forms a hydrophobic interaction with |
| | | | | CD39-V95 which is contacting Y54 as well. |
| E140 | NTD1/apex | h-bond/salt | CDR H1-H31 | E140 is a key residue of the interface. |
| | | bridge | | |
| S141 | NTD1/apex | NA | NA | Partially buried at the interface. |
| L144 | NTD1/apex | Pi-alkyl | CDR H1-H31 | L144 is totally buried at the interface and it likely |
| | | | | forms a Pi-alkyl interaction with CDR H1-H31. |
| R147 | NTD1/apex | Pi-alkyl | CDR H3-Y101 | R147 is exposed at the molecular surface and is |
| | | | | oriented toward the Ab/Ag interface. It may forms a |
| | | | | Pi-alkyl interaction with CDR H3-Y101 |
| D150 | NTD1/lateral side | NA | NA | Minor contribution to the interface. |
| | | | | May form a salt bridge with R154 and play a role in |
| | | | | CD39-R154 positioning. R154 forms a h-bond with |
| | | | | CDR H3-G103 |
| V151 | NTD1/lateral side | Alkyl | CDR H3-V106 | V151 is almost completely buried at the interface in |
| | | | | front of the CDR H3. It forms hydrophobic |
| | | | | interactions with CDR H3-V106. |
| R154 | NTD1/lateral side | h-bond | CDR H3-G103 | R154 is oriented toward the interface and interacts |
| | | | | directly with CDR H3 (at the top of the loop). |
| S294 | CTD2/apex | Potential | FR3-L72 | S294 is almost completely buried at the interface |
| | | h-bond | | and may forms a h-bond with the Calpha part of |
| | | | | FR3-L72 |
| D295 | CTD2/apex | NA | NA | Partially buried at the interface. No specific |
| | | | | comment. |
| Y296 | CTD2/apex | NA | NA | Minor contribution to the interface. |
| K298 | CTD2/groove | Potential | CDR H2-T55 | K298 is partially buried at the interface and may |
| | entry (front) | h-bond | | forms a h-bond with CDR H2-T55. |
| P300 | CTD2/groove | NA | NA | Minor contribution to the interface. |
| | entry (back) | | | |
| E306 | CTD2/apex | NA | NA | All these residues show a minor contribution to the |
| T308 | | | | interface. They are located in front of the VH |
| Q312 | | | | domain lateral side (FR3-L72-D73-T74-S75) |

TABLE 7
Residues in VH that bind the CD39 N292 glycan

| Kabat | | Type | |
|---|---|---|---|
| position | Location | of | Target moiety on |
| in VH | in VH | bond | CD39-N292 glycan |
| K19 | FR1 | NA | NAG31 |
| P57 | CDR H2 | NA | NAG31 |
| Y59 | CDR H2 | h-bond | NAG32 |
| G65 | CDR H2 | NA | NAG32 |
| F67 | FR3 | NA | NAG32 |
| V68 | FR3 | NA | NAG31 |
| | | | NAG32 |
| | | | FUC33 |
| F69 | FR3 | NA | NAG31 |
| | | | NAG32 |
| S70 | FR3 | NA | NAG31 |
| | | h-bond | FUC33 |
| L71 | FR3 | NA | FUC33 |
| Q78 | FR3 | NA | FUC33 |

### Example 9: Fc Mutations that Increase the Stability of Antibodies with High Hydrophobicity

Antibody I-392 of Example 8 (having the VH and VL CDRs of I-392 (the parental VH and VL of SEQ ID NOS: 8 and 9 modified by introduction of human VH and VL acceptor frameworks) was produced as a human IgG1 in a variety of different variants having different mutations in the heavy chain constant regions that each caused a reduction and/or loss of binding to human Fc receptors while retaining CD39 binding. The VH and Vk sequences of each antibody were cloned into vectors containing the huIgG1 CH1 constant domain and the huCk constant domain respectively. The two obtained vectors were co-transfected into the CHO cell line.

All antibodies were tested for stability in the following reference formulation at a concentration of approximatively 7 mg/mL: pH 6.0; histidine buffer (10 mM); sucrose (200 mM); NaCl (50 mM); Polysorbate 80 (PS80) (0.2 g/L). A high concentration of PS80 was tested separately at 0.5 g/L, however this did not permit a reduction in the macroscopic aggregation of the I-392 antibody. So for this study the concentration is set at 0.2 g/L.

The stability of the formulations was monitored in two storage conditions (at +5° C.±3° C. and at +40±3° C. For each study, 3 times point were performed: TO, T15D (15 days) and T1M (1 month). A freeze thaw (F/T) and a thermal shift stability assay (TSSA) were conducted for the format comparison. To perform F/T cycles, the samples were frozen at least 2 hours at −20° C. and thawed at least 1 hour at room temperature, the F/T cycle is repeated three times and samples are tested 24h after the last Freeze/Thaw cycle. At each time point, the following tests were performed:
Particulate Matter (MFI)
Visual Inspection (Appearance)
Impurities (SE-HPLC)
Turbidity (400 nm)
Protein Concentration (280 nm) (performed with Nanodrop, Thermo Fisher Scientific Inc.)

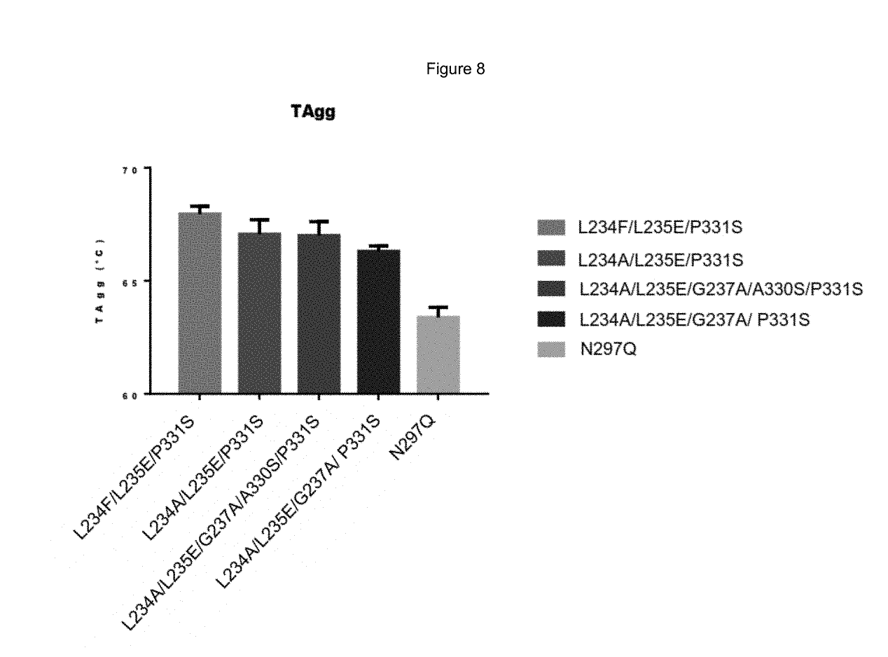

As shown in FIG. 8, several mutants showed a higher aggregation temperature (TAgg) compared to the N297Q mutant of human IgG1. Aggregation temperature is correlated with the intrinsic stability. The higher the TAgg, the higher the stability of the protein. Surprisingly, Fc domain variants with mutations in the hinge displayed high stability, and moreover improved the stability of the antibody compared to the N297Q variant, and furthermore stability was improved compared to the parental mouse antibody and to the antibody as a human IgG4, the latter displaying particularly low stability (aggregation). The stability of each of the Fc domain of human IgG1 isotype comprises an amino acid substitution at Kabat residue(s) 234, 235, 237, 330 and/or 331 (L234A/L235E/P331S substitutions, L234F/L235E/P331S substitutions, L234A/L235E/G237A/P331S substitutions, and L234A/L235E/G237A/A330S/P331S substitutions) improved the antibody, as shown in the table below. Such mutations can therefore enhance the pharmacological properties and/or activity of the antibody.

| Format | T_{agg} | T_{agg} | T_{agg} | | T_{agg} |
|---|---|---|---|---|---|
| (human IgG1 Fc mutations) | run1 | run2 | run3 | SD | Mean |
| L234F/L235E/P331S | 67.65 | 67.87 | 68.34 | 0.35 | 68.0 |
| L234A/L235E/P331S | 66.50 | 66.91 | 67.77 | 0.65 | 67.1 |
| L234A/L235E/G237A/A330S/P331S | 66.35 | 67.58 | 67.07 | 0.62 | 67.0 |
| L234A/L235E/G237A/P331S | 66.08 | 66.55 | 66.29 | 0.24 | 66.3 |
| N297Q | 63.41 | 62.91 | 63.81 | 0.45 | 63.4 |

SD = Standard Deviation
TAgg = Temperature of Aggregation

The amino acid sequence of the mutated Fc domains that increased antibody stability are shown below:

1. L234F/L235E/P331S mutation
(SEQ ID NO: 22)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E F E G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K
2. L234A/L235E/P331S mutation
(SEQ ID NO: 21)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G G P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K
3. L234A/L235E/G237A/A330S/P331S mutation:
(SEQ ID NO: 23)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E [symbol] E G A P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P S S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K
4. L234A/L235E/G237A/P331S mutation
(SEQ ID NO: 24)
A S T K G P S V F P L A P S S K S T S G G T A A L
G C L V K D Y F P E P V T V S W N S G A L T S G V
H T F P A V L Q S S G L Y S L S S V V T V P S S S
L G T Q T Y I C N V N H K P S N T K V D K R V E P
K S C D K T H T C P P C P A P E A E G A P S V F L
F P P K P K D T L M I S R T P E V T C V V V D V S
H E D P E V K F N W Y V D G V E V H N A K T K P R
E E Q Y N S T Y R V V S V L T V L H Q D W L N G K
E Y K C K V S N K A L P A S I E K T I S K A K G Q
P R E P Q V Y T L P P S R E E M T K N Q V S L T C
L V K G F Y P S D I A V E W E S N G Q P E N N Y K
T T P P V L D S D G S F F L Y S K L T V D K S R W
Q Q G N V F S C S V M H E A L H N H Y T Q K S L S
L S P G K

I-392 displays a relatively low inherent stability which is believed to be due to the numerous aromatic amino acid residues at the surface of the mAb, located in the CDRs. As illustrated in Example 9, antibodies such as I-391 and I-392 that bind to both the N- and C-domains and act as allosteric inhibitors have an unusually high number of aromatic acid residues in their CDRs. These aromatic residues confer a relatively high predicted hydrophobicity to the antibody. However, the aromatic residues in these antibodies appear to be important for antibody function, as several of the residues are involved in important interactions with CD39 and/or VH-VL interactions and can probably not be replaced by a non-aromatic residue without a reduction or loss of activity.

The ability to improve the stability of antibodies having high hydrophobicity with Fc domain substitutions may therefore be applicable to other antibodies having high hydrophobicity and/or aromatic residues in their CDRs (e.g. anti-CD39 antibodies with multiple tyrosines in their heavy chain CDR3).

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar references are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment herein using terms such as “comprising”, “having,” “including,” or “containing” with reference to an element or elements is intended to provide support for a similar aspect or embodiment herein that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. An antibody that specifically binds human CD39 (NTPDase1) at the surface of a cell without binding to CD39-L1, -L2, -L3 or -L4 polypeptides, and that is capable of neutralizing the ATPase activity of human CD39 (NTPDase1) without substantially inducing or increasing the internalization of cell surface CD39, wherein the antibody substantially lacks binding to human CD16, CD32a, CD32b and CD64 polypeptides wherein the V_{H} comprises:
(a) a CDR1 capable of contacting the N-terminal domain of CD39, wherein the residues at Kabat position 31, 32 and 33 have the formula X₁ X₂ X₃, wherein X₁ represents a histidine or asparagine, X₂ represents an aromatic residue, and X₃ represents glycine or another amino acid that avoids steric hindrance;
(b) a CDR2-FR3 segment capable of contacting the C-terminal domain of CD39, wherein the residues at Kabat position 59-71 have the formula X₁ X₂ X₃ X₄ X₅ X₆ X₇ X₈ X₉ X₁₀ X₁₁ X₁₂ X₁₃ (SEQ ID NO: 12), wherein X₁ represents a tyrosine, each of X₂, X₃, X₄, X₅ and X₆ each represent any amino acid, X₇ represents glycine or another residue which does not introduce steric hindrance that reduces antigen binding, X₈ represents any amino acid, X₉ represents phenylalanine or another hydrophobic residue capable of maintaining the beta-strand position and V_{H} domain structure integrity, X₁₀ represents alanine, valine, leucine, threonine, or a hydrophobic residue, X₁₁ represents phenylalanine or another hydrophobic residue capable of maintaining the beta-strand position and V_{H} domain structure integrity and X₁₂ represents serine, wherein and X₁₃ represents leucine, alanine, valine, threonine or arginine; and
(c) a CDR3 capable of contacting the N-terminal of CD39, wherein the residues at Kabat position 100 to 100f, to the extent residues are present at these positions, comprise a sequence of amino residues having the formula X₁ X₂ X₃ X₄ X₅ (SEQ ID NO: 15), wherein any two, three or more of X₁, X₂, X₃, X₄ and X₅ represent an aromatic amino acid; and
wherein the antibody comprises an Fc domain of human IgG1 isotype comprises an amino acid substitution at Kabat residue(s) 234, 235, 237, 330 and/or 331.

2. (canceled)

3. (canceled)

4. (canceled)

5. The antibody of claim 1, wherein the antibody comprises a V_{H} that binds CD39 and a V_{L} that binds to the Kabat CDR3 of the V_{H}, and wherein the Kabat CDR3 of the V_{H} comprises at least 5, or 6 aromatic amino acid residues.

6. (canceled)

7. The antibody of claim 1, wherein the Fc domain comprises L234A/L235E/P331S substitutions, L234F/L235E/P331S substitutions, L234A/L235E/G237A/P331S substitutions, or L234A/L235E/G237A/A330S/P331S substitutions.

8. The antibody of claim 1, wherein the antibody comprises a V_{H} and a V_{L} domain, wherein the V_{H} comprises a first antigen binding domain that is capable of binding to the N-terminal domain of CD39 and a second antigen binding domain that that is capable of binding to amino acid residues of the C-terminal domain of CD39.

9. (canceled)

10. (canceled)

11. (canceled)

12. (canceled)

13. The antibody of claim 1, wherein the V_{H} comprises a human acceptor framework.

14. The antibody of claim 1, wherein the V_{L} comprises a human acceptor framework and a CDR1, CDR2 and CDR3, wherein at least one of the CDRs contacts the CDR3 of the V_{H}, optionally wherein each of CDR1, CDR2 and CDR3 contact the CDR3 of the V_{H}.

15. The antibody of claim 1, wherein the V_{L} comprises a human acceptor framework and
a CDR1 comprising a residue at Kabat positions 31, 32, 33 and/or 34 capable of contacting the CDR3 of the V_{H};
a FR2 comprising an aromatic residue at Kabat position 49 capable of contacting the CDR3 of the V_{H};
a CDR2 comprising a residue at Kabat position 50 capable of contacting the CDR3 of the V_{H}; and
a CDR3 comprising a residue at Kabat positions 89 and/or 91 capable of contacting the CDR3 of the V_{H}.

16. The antibody of claim 1, wherein the V_{L} comprises a human acceptor framework and
a CDR1 wherein the residues at Kabat position 31, 32, 33 and 34 have the formula TX₁VA, wherein X₁ represents alanine or asparagine; or the formula SYX₁X₂, wherein X₁ represents a hydrophobic residue, and X₂ represents any amino acid;
a FR2 comprising an aromatic residue, at Kabat position 49;
a CDR2 wherein the residue at Kabat position 50 is a serine, lysine or threonine; and
a CDR3 wherein a glutamine or histidine is present Kabat position 89 and/or 90, the residue at Kabat position 91 is a tyrosine, threonine or histidine.

17. The antibody of claim 16, wherein the V_{L} comprises a residue at Kabat FR3 positions 94, 95, 96 and/or 97 capable of contacting the CDR3 of the V_{H}.

18. (canceled)

19. The antibody of claim 1, wherein the antibody has reduced binding to a mutant CD39 polypeptide comprising a mutation at 1, 2, 3 or 4 residues selected from the group consisting of Q96, N99, E143 and R147 with reference to SEQ ID NO: 1, in each case relative to binding between the antibody and a wild-type CD39 polypeptide comprising the amino acid sequence of SEQ ID NO: 1.

20. (canceled)

21. (canceled)

22. (canceled)

23. (canceled)

24. (canceled)

25. (canceled)

26. The antibody of claim 1, wherein the antibody is capable of causing a decrease in the extracellular ATPase activity by a B cell by at least 80%.

27. The antibody of claim 1, wherein the antibody is characterized by an EC₅₀ for neutralization of ATPase activity of cellular CD39 of no more than 1 μg/ml, wherein neutralization of the enzymatic activity of CD39 is determined by assessing neutralization of ATPase activity on RAMOS cells by quantifying hydrolysis of ATP to AMP.

28. (canceled)

29. The antibody of claim 1, wherein the antibody is capable of causing a decrease in the ATPase activity of human membrane-bound CD39 polypeptide by more than 70%.

30. A monoclonal antibody characterized by:
a) specifically binding with high affinity to, and/or neutralizing the ATPase activity of human “vascular” CD39 polypeptide expressed at the surface of a cell;
b) not inducing or increasing down-modulation and/or internalization of the antibody-CD39 complex;
c) not binding to soluble human CD39 polypeptide, or L4 isoforms; and
d) not binding via its Fc domain to the CD16 human Fcγ receptor, CD16A, CD16B, CD32A, CD32B and CD64.

31. (canceled)

32. The antibody of claim 30, wherein the antibody competes for binding to a CD39 polypeptide of SEQ ID NO: 1 with an antibody comprising the heavy and light chain CDRs, or the heavy and light chain variable regions of antibody I-391.

33. (canceled)

34. (canceled)

35. (canceled)

36. The antibody of claim 1, wherein the antibody comprises a V_{H} and a V_{L}, wherein the V_{H} comprises the amino acid sequence of Formula I:
[FR₁]CDR1[FR₂]CDR2[FR₃]CDR3[FR₄] (Formula I)
wherein [FR₁], [FR₂], [FR₃] and [FR₄] represent human V_{H} framework regions and CDR1, CDR2 and CDR3 represent V_{H} CDRs, wherein:
CDR1 comprises a residue at Kabat positions 31 and/or 33, that is capable of contacting the N-terminal domain of CD39,
CDR2 comprises residues capable of contacting the N-terminal domain of CD39, wherein two, three, four or five of Kabat positions 50, 52, 52a, 53 and 56 are capable of contacting CD39 wherein the residue at position 53 comprises an aromatic ring further wherein a residue in the Kabat CDR2, in combination with residues in the Kabat FR3 are capable of contacting the C-terminal domain of CD39,
CDR3 comprises an aromatic residue capable of contacting the N-terminal domain of CD39, wherein the CDR3 further comprises an aromatic residue capable of contacting the V_{L}, wherein the aromatic residue(s) is/are at any of Kabat positions 100, 100b, 100c, 100d, 100e and/or 100f to the extent residues are present at the particular position, wherein the aromatic residue capable of contacting the V_{L} is a tyrosine or a phenylalanine and wherein the aromatic residue capable of contacting CD39 is a tyrosine or a phenylalanine; and
wherein the V_{L} comprises the amino acid sequence of Formula II:
[FR₁]CDR1[FR₂]CDR2[FR₃]CDR3[FR₄] (Formula II)
wherein [FR₁], [FR₂], [FR₃] and [FR₄] represent human V_{L} framework regions and CDR1, CDR2 and CDR3 represent V_{L} CDRs, wherein:
CDR1 comprises a residue at Kabat positions 31, 32, 33 and/or 34, capable of contacting the CDR3 of the V_{H};
FR2 comprises an aromatic residue at Kabat position 49, capable of contacting the CDR3 of the V_{H}; and
CDR3 comprises a residue at Kabat positions 89 and/or 91, capable of contacting the CDR3 of the V_{H}.

37. (canceled)

38. The antibody claim 30, wherein the V_{H} comprises:
(a) a CDR1 capable of contacting the N-terminal domain of CD39 wherein the residues at Kabat position 31, 32 and 33 have the formula X₁ X₂ X₃, wherein X₁ represents a histidine or asparagine, X₂ represents an aromatic residue, and X₃ represents glycine, or another amino acid that avoids steric hindrance;
(b) a CDR2-FR3 segment capable of contacting the C-terminal domain of CD39, wherein the residues at Kabat position 50-71 having the formula X₁ X₂ X₃ X₄ X₅ X₆ X₇ X₈ X₉ X₁₀ X₁₁ X₁₂ X₁₃ X₁₄ X₁₅ X₁₆ X₁₇ X₁₈ X₁₉ X₂₀ X₂₁ X₂₂ X₂₃ (SEQ ID NO: 14), wherein X₁ represents tryptophan, X₂ represents any amino acid, X₃ represents asparagine or glutamine, X₄ represents threonine, X₅ represents any amino acid residue, X₆ represents any amino acid, X₇ represents any amino acid, X₈ represents glutamic acid aspartic acid, X₉ represents any amino acid, X₁₀ represents any amino acid, X₁₁ represents a tyrosine, each of X₁₂, X₁₃, X₁₄, X₁₅ and X₁₆ each represent any amino acid, X₁₇ represents glycine or another residue which does not introduce steric hindrance that reduces antigen binding, X₁₈ represents any amino acid, X₁₉ represents phenylalanine or another hydrophobic residue capable of maintaining the beta-strand position and V_{H} domain structure integrity, X₂₀ represents alanine, valine, leucine, or a hydrophobic residue, X₂₁ represents phenylalanine or another hydrophobic residue capable of maintaining the beta-strand position and V_{H} domain structure integrity and X₂₂ represents serine, wherein and X₂₃ represents any amino acid, wherein the CDR2-FR3 segment further comprises residues at Kabat positions 72, 72a and 72b having the formula X₂₄ X₂₅ X₂₆, wherein X₂₄ represents aspartic acid, glutamic acid or alanine, X₂₅ represents any amino acid, and X₂₆ represents serine, or alanine; and
(c) a CDR3 capable of contacting the N-terminal of CD39 and capable of contacting the N-terminal domain of CD39 and the V_{L}, wherein the residues at Kabat position 95-102 have the formula X₁ X₂ X₃ X₄ X₅ X₆ X₇ X₈ X₉ X₁₀ X₉ X₁₀ X₁₁ X₁₂ X₁₃ X₁₄ (SEQ ID NO: 16), wherein
X₁ represents arginine or lysine,
X₂ represents any amino acid,
X₃ represents any amino acid residue,
X₄ represents any amino acid,
X₅ represents glycine or arginine,
X₆ represents any amino acid,
X₇ represents any amino acid,
X₈ represents valine, alanine, isoleucine, leucine, or an aromatic amino acid,
X₉ represents any amino acid,
X₁₀ represents tyrosine, phenylalanine, or methionine,
X₁₁ is absent or represents any amino acid,
X₁₂ is absent or represents any amino acid,
X₁₃ represents any amino acid,
X₁₄ represents any amino acid.

39. The antibody of claim 38, wherein the V_{H} comprises a human acceptor framework.

40. (canceled)

41. (canceled)

42. The antibody of claim 36, wherein the V_{L} comprises a human acceptor framework and
a CDR1 wherein the residues at Kabat position 31, 32, 33 and 34 have (a) the formula TX₁VA, wherein X₁ represents alanine or asparagine; or (b) the formula SYX₁X₂, wherein X₁ represents a hydrophobic residue, and X₂ represents any amino acid;
a FR2 comprising an aromatic residue, at Kabat position 49;
a CDR2 wherein the residue at Kabat position 50 is a serine or threonine; and
a CDR3 wherein a glutamine or histidine is present Kabat position 89 and/or 90, the residue at Kabat position 91 is a tyrosine, threonine or histidine, wherein the residue at position 95 is a proline, wherein the residue at position 96 is an aromatic residue.

43. The antibody of claim 30, wherein the antibody binds an epitope on CD39 comprising an amino acid residue selected from the group consisting of Q96, N99, E143 and R147 with reference to SEQ ID NO: 1.

44. The antibody of claim 30, wherein the antibody has reduced binding to a mutant CD39 polypeptide comprising a mutation at 1, 2, 3 or 4 residues selected from the group consisting of Q96, N99, E143 and R147 with reference to SEQ ID NO: 1, in each case relative to binding between the antibody and a wild-type CD39 polypeptide comprising the amino acid sequence of SEQ ID NO: 1.

45. (canceled)

46. (canceled)

47. (canceled)

48. (canceled)

49. (canceled)

50. A pharmaceutical composition comprising an antibody according to claim 1, and a pharmaceutically acceptable carrier.

51. (canceled)

52. A nucleic acid encoding a heavy and/or light chain of an antibody of claim 1.

53. A recombinant host cell producing the antibody of claim 1.

54. A method for the treatment or prevention of cancer in a patient in need thereof, the method comprising administering to said patient an effective amount of an antibody of claim 1.

55. (canceled)

56. (canceled)

57. (canceled)

58. (canceled)

59. (canceled)

60. (canceled)

61. The method of claim 54, wherein the method comprises administering to said individual an effective amount of an antibody of claim 1, in combination with an antibody that neutralizes the inhibitory activity of human PD-1.

62. (canceled)

63. (canceled)

64. (canceled)

65. (canceled)

66. (canceled)

67. (canceled)

68. (canceled)

69. A method of producing or testing an antibody which binds and neutralizes the enzymatic activity of CD39 without inducing or increasing down-modulation of CD39 cell surface expression, said method comprising the steps of:
(a) providing a plurality of antibodies that bind a CD39 polypeptide,
(b) bringing each of said antibodies into contact with a mutant CD39 polypeptide comprising a mutation at 1, 2, 3 or 4 residues selected from the group consisting of Q96, N99, E143 and R147 with reference to SEQ ID NO: 1, and assessing binding between the antibody and the mutant CD39 polypeptide, relative to binding between the antibody and a wild-type CD39 polypeptide comprising the amino acid sequence of SEQ ID NO: 1, and
(c) selecting an antibody that has reduced binding to the mutant CD39 polypeptide, relative to binding between the antibody and a wild-type CD39 polypeptide comprising the amino acid sequence of SEQ ID NO: 1.

70. The method of claim 69, further comprising the steps of:
(a) bringing each of the antibodies selected in step (c) of claim 69 into contact with CD39-expressing cells;
(b) assessing production of AMP by mass spectrometry, wherein a decrease in AMP generated indicates neutralization of ATPase activity; and
(c) selecting an antibody that results in a decrease of AMP generated by at least 70%.
